(19) United States
(12) Patent Application Publication
(10) Pub. No.: US 2023/0148599 A1
(43) Pub. Date: May 18, 2023

(54) FUNGICIDAL MIXTURES CONTAINING PYRAZOLE DERIVATIVES

(71) Applicant: FMC Corporation, Philadelphia, PA (US)

(72) Inventors: Jeffrey Keith LONG, Wilmington, DE (US); Srinivas CHITTABOINA, Bibipet (IN); Travis Chandler MCMAHON, Middletown, DE (US)

(21) Appl. No.: 17/910,051

(22) PCT Filed: Mar. 11, 2021
(86) PCT No.: PCT/US21/21826

§ 371 Date: Sep. 8, 2022

Related U.S. Application Data
(60) Provisional application No. 62/988,128, filed on Mar. 11, 2020.

(51) Int. Cl. A01N 43/56; A01P 3/00; C07D 231/38

(52) U.S. Cl. CPC A01N 43/56; A01P 3/00; C07D 231/38

(57) ABSTRACT

Disclosed is a fungicidal composition comprising (a) at least one compound selected from the compounds of Formula 1, including all geometric and stereoisomers, tautomers, iV-oxides, and salts thereof, (I) wherein R¹, R², R³, R⁴, R⁵, R⁶, m and n are as defined in the disclosure and (b) at least one additional fungicidal compound. Also disclosed is a method for controlling plant diseases caused by fungal plant pathogens comprising applying to the plant or portion thereof, or to the plant seed, a fungicidally effective amount of a compound of Formula 1, an A-oxide, or salt thereof (e.g., as a component in the aforesaid composition). Also disclosed is a composition comprising: (a) at least one compound selected from the compounds of Formula 1 described above, A-oxides, and salts thereof; and at least one invertebrate pest control compound or agent.

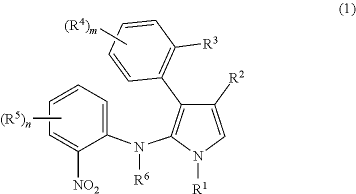

### FIELD OF THE INVENTION

This invention relates to certain pyrazoles, their N-oxides, salts and to mixtures and compositions comprising such halomethyl ketone and hydrate derivatives and methods for using such halomethyl ketone and hydrate derivatives and their mixtures and compositions as fungicides.

### BACKGROUND OF THE INVENTION

The control of plant diseases caused by fungal plant pathogens is extremely important in achieving high crop efficiency. Plant disease damage to ornamental, vegetable, field, cereal and fruit crops can cause significant reduction in productivity and thereby result in increased costs to the consumer. In addition to often being highly destructive, plant diseases can be difficult to control and may develop resistance to commercial fungicides. Many products are commercially available for these purposes, but the need continues for new fungicidal compounds which are more effective, less costly, less toxic, environmentally safer or have different sites of action. Besides introduction of new fungicides, combinations of fungicides are often used to facilitate disease control, to broaden spectrum of control and to retard resistance development. Furthermore, certain rare combinations of fungicides demonstrate a greater-than-additive (i.e. synergistic) effect to provide commercially important levels of plant disease control. The advantages of particular fungicide combinations are recognized in the art to vary, depending on such factors as the particular plant species and plant disease to be treated, and whether the plants are treated before or after infection with the fungal plant pathogen. Accordingly, new advantageous combinations are needed to provide a variety of options to best satisfy particular plant disease control needs. Such combinations have now been discovered.

PCT Patent Publications WO 2018/052838, WO 2013/192126, WO 2012/031061 and WO 2010/101973 disclose fungicidal pyrazoles and their use in agriculture. PCT Patent Publication WO 2019/020981 discloses pyrazole, isothiazole and isoxazole derivatives and their use in agriculture.

### SUMMARY OF THE INVENTION

This invention relates to a fungicidal composition (i.e. combination) comprising (a) at least one compound selected from the compounds of Formula 1 (including all stereoisomers), N-oxides, and salts thereof:

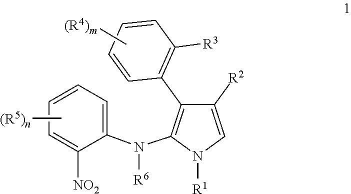

wherein
R¹ is C₁-C₂ alkyl;
R² is cyano, halogen, C₁-C₂ alkyl or C₁-C₂ haloalkyl;
R³ is halogen or methyl;
each R⁴ is independently halogen, cyano, nitro, C₁-C₃ alkyl, C₁-C₃ alkoxy, C₁-C₃ haloalkoxy, C₂-C₆ alkenyloxy, C₂-C₆ alkynyloxy, C₂-C₆ cyanoalkoxy, C₂-C₆ alkoxyalkyl or C₂-C₆ alkoxyalkoxy;
each R⁵ is independently halogen, C₁-C₃ alkyl, C₂-C₆ alkoxyalkyl, C₁-C₃ alkoxy, C₁-C₃ haloalkoxy, C₂-C₆ alkenyloxy, C₂-C₆ alkynyloxy, C₂-C₆ cyanoalkoxy or C₂-C₆ alkoxyalkoxy;
m and n are each independently 0, 1, 2 or 3;
R⁶ is H; or C₁-C₃ alkyl or C₁-C₃ haloalkyl, each optionally substituted with up to 2 substituents independently selected from R^{6a}; or amino, C₂-C₄ alkenyl, C₂-C₄ alkynyl, C₃-C₆ cycloalkyl, CH(═O), S(═O)₂OM, S(═O)ᵤR⁷, (C═W)R⁸ or OR⁹;
each R^{6a} is independently cyano, C₃-C₆ cycloalkyl, C₁-C₃ alkoxy, C₁-C₃ haloalkoxy, C₁-C₃ alkylthio, C₁-C₃ alkylsulfinyl or C₁-C₃ alkylsulfonyl; M is K or Na;
u is 0, 1 or 2;
R⁷ is C₁-C₃ alkyl or C₁-C₃ haloalkyl;
W is O or S;
R⁸ is C₁-C₃ alkyl, C₂-C₄ alkoxyalkyl, C₂-C₄ alkylaminoalkyl, C₃-C₆ dialkylaminoalkyl, C₁-C₃ alkoxy, C₁-C₃ alkylthio or C₂-C₄ alkylthioalkyl;
R⁹ is H; or C₁-C₃ alkyl or C₁-C₃ haloalkyl, each optionally substituted with up to 2 substituents independently selected from R^{9a}; or CH(═O), C₃-C₆ cycloalkyl, S(═O)₂OM or (C═W)R¹⁰;
each R^{9a} is independently cyano, C₃-C₆ cycloalkyl, C₁-C₃ alkoxy, C₁-C₃ haloalkoxy, C₁-C₃ alkylthio, C₁-C₃ alkylsulfinyl or C₁-C₃ alkylsulfonyl; and
R¹⁰ is C₁-C₃ alkyl, C₂-C₄ alkoxyalkyl, C₂-C₄ alkylaminoalkyl, C₃-C₆ dialkylaminoalkyl, C₁-C₃ alkoxy, C₁-C₃ alkylthio or C₂-C₄ alkylthioalkyl; and
(b) at least one additional fungicidal compound;
   provided that the compound of Formula 1 is not:
4-(2,6-difluoro-4-methoxyphenyl)-N-(2,4-difluoro-6-nitrophenyl)-1,3-dimethyl-1H-pyrazol-5-amine;
4-(2-chloro-4-fluorophenyl)-1,3-dimethyl-N-(2-nitrophenyl)-1H-pyrazol-5-amine;
4-(2-chloro-4-fluorophenyl)-N-(2,4-difluoro-6-nitrophenyl)-1,3-dimethyl-1H-pyrazol-5-amine;
4-(2-chloro-4-fluorophenyl)-3-ethyl-1-methyl-N-(2-nitrophenyl)-1H-pyrazol-5-amine;
4-(2-chloro-4-fluorophenyl)-1-methyl-N-(2-nitrophenyl)-3-(trifluoromethyl)-1H-pyrazol-5-amine;
4-(2,6-difluoro-4-methoxyphenyl)-N-(2-methoxy-6-nitrophenyl)-1,3-dimethyl-1H-pyrazol-5-amine;
4-(2-chloro-4-fluorophenyl)-N-(2-methoxy-6-nitrophenyl)-1,3-dimethyl-1H-pyrazol-5-amine;
N-(2-chloro-6-nitrophenyl)-4-(2,6-difluoro-4-methoxyphenyl)-1,3-dimethyl-1H-pyrazol-5-amine;
N-(2-chloro-3-fluoro-6-nitrophenyl)-4-(2,6-difluoro-4-methoxyphenyl)-1,3-dimethyl-1H-pyrazol-5-amine;
4-(2-chloro-4-fluorophenyl)-1,3-dimethyl-N-(2-methyl-6-nitrophenyl)-1H-pyrazol-5-amine;
N-(2-bromo-4-fluoro-6-nitrophenyl)-4-(2-chloro-4-fluorophenyl)-1,3-dimethyl-1H-pyrazol-5-amine;
4-(2-chloro-4-fluorophenyl)-N-(4-methoxy-2-nitrophenyl)-1,3-dimethyl-1H-pyrazol-5-amine;
4-(2,6-difluoro-4-methoxyphenyl)-N-(4-fluoro-2-nitrophenyl)-1,3-dimethyl-1H-pyrazol-5-amine;
4-(2,6-difluoro-4-methoxyphenyl)-N-(4-methoxy-2-nitrophenyl)-1,3-dimethyl-1H-pyrazol-5-amine;
N-(4-chloro-2-nitrophenyl)-4-(2,6-difluoro-4-methoxyphenyl)-1,3-dimethyl-1H-pyrazol-5-amine;
4-(2,6-difluoro-4-methoxyphenyl)-1,3-dimethyl-N-[2-nitro-4-(2-propyn-1-yloxy) phenyl]-1H-pyrazol-5-amine;
4-(2,6-difluoro-4-methoxyphenyl)-1,3-dimethyl-N-[2-nitro-4-(2-propen-1-yloxy) phenyl]-1H-pyrazol-5-amine;
N-(4-bromo-2-nitrophenyl)-4-(2,6-difluoro-4-methoxyphenyl)-1,3-dimethyl-1H-pyrazol-5-amine;
N-(4-chloro-2-fluoro-6-nitrophenyl)-4-(2,6-difluoro-4-methoxyphenyl)-1,3-dimethyl-1H-pyrazol-5-amine;
3-chloro-4-(2-chloro-4-fluorophenyl)-N-(2,4-difluoro-6-nitrophenyl)-1-methyl-1H-pyrazol-5-amine;
4-(2,6-difluoro-4-methoxyphenyl)-1,3-dimethyl-N-[4-methyl-2-nitrophenyl]-1H-pyrazol-5-amine;
4-(2-chloro-4-fluorophenyl)-1,3-dimethyl-N-(4-methyl-2-nitrophenyl)-1H-pyrazol-5-amine; and
N-(4-bromo-2-fluoro-6-nitrophenyl)-4-(2,6-difluoro-4-methoxyphenyl)-1,3-dimethyl-1H-pyrazol-5-amine.

This invention also relates to a composition comprising: (a) at least one compound selected from the compounds of Formula 1 described above, N-oxides, and salts thereof; and at least one invertebrate pest control compound or agent.

This invention also relates to a composition comprising one of the aforesaid compositions comprising component (a) and at least one additional component selected from the group consisting of surfactants, solid diluents and liquid diluents.

This invention also relates to a method for controlling plant diseases caused by fungal plant pathogens comprising applying to the plant or portion thereof, or to the plant seed, a fungicidally effective amount of one of the aforesaid compositions.

The aforedescribed method can also be described as a method for protecting a plant or plant seed from diseases caused by fungal pathogens comprising applying a fungicidally effective amount of one of the aforesaid compositions to the plant (or portion thereof) or plant seed (directly or through the environment (e.g., growing medium) of the plant or plant seed).

This invention also relates to a compound of Formula 1 described above, or an N-oxide or salt thereof.

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated.

For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant.

An “increase in crop yield” refers to an increase in crop yield relative to an untreated control crop plant.

The term “biologically effective amount” refers to the amount of a biologically active compound (e.g., a compound of Formula 1) sufficient to produce the desired biological effect when applied to (i.e. contacted with) a fungus to be controlled or its environment, or to a plant, the seed from which the plant is grown, or the locus of the plant (e.g., growth medium) to protect the plant from injury by the fungal disease or for other desired effect (e.g., increasing plant vigor).

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As referred to in this disclosure, the terms “fungal pathogen” and “fungal plant pathogen” include pathogens in the Ascomycota, Basidiomycota and Zygomycota phyla, and the fungal-like Oomycota class that are the causal agents of a broad spectrum of plant diseases of economic importance, affecting ornamental, turf, vegetable, field, cereal and fruit crops. In the context of this disclosure, “protecting a plant from disease” or “control of a plant disease” includes preventative action (interruption of the fungal cycle of infection, colonization, symptom development and spore production) and/or curative action (inhibition of colonization of plant host tissues).

As used herein, the term “mode of action” (MOA) is as define by the Fungicide Resistance Action Committee (FRAC), and is used to distinguish fungicides according to their biochemical mode of action in the biosynthetic pathways of plant pathogens, and their resistance risk. FRAC-defined modes of actions include (A) nucleic acids metabolism, (B) cytoskeleton and motor protein, (C) respiration, (D) amino acids and protein synthesis, (E) signal transduction, (F) lipid synthesis or transport and membrane integrity or function, (G) sterol biosynthesis in membranes, (H) cell wall biosynthesis, (I) melanin synthesis in cell wall, (P) host plant defense induction, (U) unknown mode of action, (M) chemicals with multi-site activity and (BM) biologicals with multiple modes of action. Each mode of action (i.e. letters A through BM) contain one or more subgroups (e.g., A includes subgroups A1, A2, A3 and A4) based either on individual validated target sites of action, or in cases where the precise target site is unknown, based on cross resistance profiles within a group or in relation to other groups. Each of these subgroups (e.g., A1, A2, A3 and A4) is assigned a FRAC code (a number and/or letter). For example, the FRAC code for subgroup A1 is 4.

Additional information on target sites and FRAC codes can be obtained from publicly available databases maintained, for example, by FRAC.

As used herein, the term “cross resistance” refers to the phenomenon that occurs when a pathogen develops resistance to one fungicide and simultaneously becomes resistant to one or more other fungicides. These other fungicides are typically, but not always, in the same chemical class or have the same target site of action, or can be detoxified by the same mechanism.

Generally when a molecular fragment (i.e. radical) is denoted by a series of atom symbols (e.g., C, H, N, O and S) the implicit point or points of attachment will be easily recognized by those skilled in the art. In some instances herein, particularly when alternative points of attachment are possible, the point or points of attachment may be explicitly indicated by a hyphen (“-”). For example, “—NCS” indicates that the point of attachment is the nitrogen atom (i.e. isothiocyanato, not thiocyanato).

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified, for example, for R⁵.

In the above recitations, the term “alkyl”, used either alone or in compound words such as “alkylthio” or “haloalkyl” includes straight-chain or branched alkyl such as methyl, ethyl, n-propyl and i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, i-propyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkenyloxy” includes straight-chain or branched alkenyl attached to and linked through an oxygen atom. Examples of “alkenyloxy” include H₂C═CHCH₂O, (CH₃)₂C═CHCH₂O, CH₃CH═CHCH₂O, CH₃CH═C(CH₃)CH₂O and CH₂═CHCH₂CH₂O. “Alkynyloxy” includes straight-chain or branched alkynyl attached to and linked through an oxygen atom. Examples of “alkynyloxy” include HC═CCH₂O, CH₃C═CCH₂O and CH₃C═CCH₂CH₂O. “Alkoxyalkoxy” denotes alkoxy substitution on another alkoxy moiety. Examples of “alkoxyalkoxy” include CH₃OCH₂O, CH₃OCH₂O and CH₃CH₂OCH₂O.

“Alkylthio” includes branched or straight-chain alkylthio moieties such as methylthio, ethylthio, and the different propylthio isomers. “Alkylthioalkyl” denotes alkylthio substitution on alkyl. Examples of “alkylthioalkyl” include CH₃SCH₂, CH₃SCH₂CH₂, CH₃CH₂SCH₂ and CH₃CH₂SCH₂CH₂. “Alkylsulfinyl” includes both enantiomers of an alkylsulfinyl group. Examples of “alkylsulfinyl” include CH₃S(═O), CH₃CH₂S(═O), CH₃CH₂CH₂S(═O) and (CH₃)₂CHS(═O). Examples of “alkylsulfonyl” include CH₃S(═O)₂, CH₃CH₂S(═O)₂, CH₃CH₂CH₂S(═O)₂ and (CH₃)₂CHS(═O)₂.

“Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂. Examples of “dialkylaminoalkyl” include (CH₃)₂NCH₂, (CH₃CH₂)₂NCH₂CH₂ and CH₃CH₂(CH₃)N CH₂CH₂.

The term “cycloalkyl” denotes a saturated carbocyclic ring consisting of between 3 to 6 carbon atoms linked to one another by single bonds. Examples of “cycloalkyl” include cyclopropyl, cyclobutyl, cyclopentyl and cyclohexyl. The term “cycloalkylalkyl” denotes cycloalkyl substitution on an alkyl group. Examples of “cycloalkylalkyl” include cyclopropylmethyl, cyclopentylethyl, and other cycloalkyl moieties bonded to straight-chain or branched alkyl groups.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The term “haloalkoxy”, and the like, are defined analogously to the term “haloalkyl”. Examples of “haloalkoxy” include CF₃O, CCl₃CH₂O, F₂CHCH₂CH₂O and CF₃CH₂O.

“Cyanoalkoxy” denotes an alkyloxy group substituted with one cyano group. Examples of “cyanoalkoxy” include NCCH₂O, NCCH₂CH₂O and CH₃CH(CN)CH₂O.

The total number of carbon atoms in a substituent group is indicated by the “Cᵢ-Cⱼ” prefix where i and j are numbers from 1 to 6. For example, C₁-C₃ alkylsulfonyl designates methylsulfonyl through propylsulfonyl; C₂ alkoxyalkyl designates CH₃OCH₂; C₃ alkoxyalkyl designates, for example, CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 2 substituents independently selected from R^{6a}” means that 0, 1 or 2 substituents can be present.

When a compound is substituted with a substituent bearing a subscript that indicates the number of said substituents can vary (e.g., (R⁴)ₘ in Formula 1 wherein m is 0 to 3), then said substituents are independently selected from the group of defined substituents, unless otherwise indicated. When a variable group is shown to be optionally attached to a position, for example (R⁴)ₘ wherein m may be 0, then hydrogen may be at the position even if not recited in the definition of the variable group.

Naming of substituents in the present disclosure uses recognized terminology providing conciseness in precisely conveying to those skilled in the art the chemical structure. For sake of conciseness, locant descriptors may be omitted. In some instances herein the point or points of attachment of substituents (e.g., R⁴ and R⁵) are indicated by locant numbers which may be different from the Chemical Abstracts naming system if the difference does not affect the meaning.

Compounds of this invention can exist as one or more stereoisomers. Stereoisomers are isomers of identical constitution but differing in the arrangement of their atoms in space and include enantiomers, diastereomers, cis- and trans-isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

Compounds of this invention can exist as one or more conformational isomers due to restricted rotation about an amide bond (e.g., C(═O)—N) in Formula 1. This invention comprises mixtures of conformational isomers. In addition, this invention includes compounds that are enriched in one conformer relative to others.

This invention comprises all stereoisomers, conformational isomers and mixtures thereof in all proportions as well as isotopic forms such as deuterated compounds.

One skilled in the art will appreciate that not all nitrogen containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides.

Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus, a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, stereoisomers, N-oxides, and salts thereof, typically exist in more than one form, therefore Formula 1 includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

As described in the Summary of the Invention, an aspect of the present invention is directed at a composition comprising (a) at least one compound selected from Formula 1, N-oxides, and salts thereof, with (b) at least one additional fungicidal compound. More particularly, Component (b) is selected from the group consisting of
(b1) methyl benzimidazole carbamate (MBC) fungicides;
(b2) dicarboximide fungicides;
(b3) demethylation inhibitor (DMI) fungicides;
(b4) phenylamide (PA) fungicides;
(b5) amine/morpholine fungicides;
(b6) phospholipid biosynthesis inhibitor fungicides;
(b7) succinate dehydrogenase inhibitor (SDHI) fungicides;
(b8) hydroxy(2-amino-)pyrimidine fungicides;
(b9) anilinopyrimidine (AP) fungicides;
(b10)N-phenyl carbamate fungicides;
(b11) quinone outside inhibitor (QoI) fungicides;
(b12) phenylpyrrole (PP) fungicides;
(b13) azanaphthalene fungicides;
(b14) cell peroxidation inhibitor fungicides;
(b15) melanin biosynthesis inhibitor-reductase (MBI-R) fungicides;
(b16a) melanin biosynthesis inhibitor-dehydratase (MBI-D) fungicides;
(b16b) melanin biosynthesis inhibitor-polyketide synthase (MBI-P) fungicides;
(b17) keto reductase inhibitor (KRI) fungicides;
(b18) squalene-epoxidase inhibitor fungicides;
(b19) polyoxin fungicides;
(b20) phenylurea fungicides;
(b21) quinone inside inhibitor (QiI) fungicides;
(b22) benzamide and thiazole carboxamide fungicides;
(b23) enopyranuronic acid antibiotic fungicides;
(b24) hexopyranosyl antibiotic fungicides;
(b25) glucopyranosyl antibiotic: protein synthesis fungicides;
(b26) glucopyranosyl antibiotic fungicides;
(b27) cyanoacetamideoxime fungicides;
(b28) carbamate fungicides;
(b29) oxidative phosphorylation uncoupling fungicides;
(b30) organo tin fungicides;
(b31) carboxylic acid fungicides;
(b32) heteroaromatic fungicides;
(b33) phosphonate fungicides;
(b34) phthalamic acid fungicides;
(b35) benzotriazine fungicides;
(b36) benzene-sulfonamide fungicides;
(b37) pyridazinone fungicides;
(b38) thiophene-carboxamide fungicides;
(b39) complex I NADH oxido-reductase inhibitor fungicides;
(b40) carboxylic acid amide (CAA) fungicides;
(b41) tetracycline antibiotic fungicides;
(b42) thiocarbamate fungicides;
(b43) benzamide fungicides;
(b44) microbial fungicides;
(b45) quinone outside inhibitor, stigmatellin binding (QoSI) fungicides;
(b46) plant extract fungicides;
(b47) cyanoacrylate fungicides;
(b48) polyene fungicides;
(b49) oxysterol binding protein inhibitor (OSBPI) fungicides;
(b50) aryl-phenyl-ketone fungicides;
(b51) host plant defense induction fungicides;
(b52) multi-site activity fungicides;
(b53) biologicals with multiple modes of action;
(b54) fungicides other than fungicides of component (a) and components (b1) through (b53); and
salts of compounds of (b1) through (b54).

Of note are embodiments wherein component (b) comprises at least one fungicidal compound from each of two different groups selected from (b1) through (b54).

“Methyl benzimidazole carbamate (MBC) fungicides (b1)” (FRAC code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

“Dicarboximide fungicides (b2)” (FRAC code 2) inhibit a mitogen-activated protein (MAP)/histidine kinase in osmotic signal transduction. Examples include chlozolinate, dimethachlone, iprodione, procymidone and vinclozolin.

“Demethylation inhibitor (DMI) fungicides (b3)” (FRAC code 3) (Sterol Biosynthesis Inhibitors (SBI): Class I) inhibit C14-demethylase, which plays a role in sterol production.

Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: piperazines, pyridines, pyrimidines, imidazoles, triazoles and triazolinthiones. The piperazines include triforine. The pyridines include buthiobate, pyrifenox, pyrisoxazole and (αS)-[3-(4-chloro-2-fluorophenyl)-5-(2,4-difluorophenyl)-4-isoxazolyl]-3-pyridinemethanol. The pyrimidines include fenarimol, nuarimol and triarimol. The imidazoles include econazole, imazalil, oxpoconazole, pefurazoate, prochloraz and triflumizole. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, ipfentrifluconazole, mefentrifluconazole, metconazole, myclobutanil, penconazole, propiconazole, quinconazole, simeconazole, tebuconazole, tetraconazole, triadimefon, triadimenol, triticonazole, uniconazole, uniconazole-P, α-(1-chlorocyclopropyl)-α-[2-(2,2-dichlorocyclopropyl)ethyl]-1H-1,2,4-triazole-1-ethanol, rel-1-[[f(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluoro-phenyl)-2-oxiranyl]methyl]-1,2-dihydro-3H-1,2,4-triazole-3-thione and rel-1-[[(2R,3S)-3-(2-chlorophenyl)-2-(2,4-difluorophenyl)-2-oxiranyl]methyl]-5-(2-propen-1-ylthio)-1H-1,2,4-triazole. The triazolinthiones include prothioconazole. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

“Phenylamide (PA) fungicides (b4)” (FRAC code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M (also known as kiralaxyl), furalaxyl, metalaxyl and metalaxyl-M (also known as mefenoxam). The oxazolidinones include oxadixyl. The butyrolactones include ofurace.

“Amine/morpholine fungicides (b5)” (FRAC code 5) (SBI: Class II) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

“Phospholipid biosynthesis inhibitor fungicides (b6)” (FRAC code 6) inhibit growth of fungi by affecting phospholipid biosynthesis. Phospholipid biosynthesis fungicides include phophorothiolate and dithiolane fungicides. The phosphorothiolates include edifenphos, iprobenfos and pyrazophos. The dithiolanes include isoprothiolane.

“Succinate dehydrogenase inhibitor (SDHI) fungicides (b7)” (FRAC code 7) inhibit complex II fungal respiration by disrupting a key enzyme in the Krebs Cycle (TCA cycle) named succinate dehydrogenase. Inhibiting respiration prevents the fungus from making ATP, and thus inhibits growth and reproduction. SDHI fungicides include phenylbenzamide, phenyloxoethylthiophene amide, pyridinylethylbenzamide, furan carboxamide, oxathiin carboxamide, thiazole carboxamide, pyrazole-4-carboxamide, N-cyclopropyl-N-benzyl-pyrazole carboxamide, N-methoxy-(phenyl-ethyl)-pyrazole carboxamide, pyridine carboxamide and pyrazine carboxamide fungicides. The phenylbenzamides include benodanil, flutolanil and mepronil. The phenyloxoethylthiophene amides include isofetamid. The pyridinylethylbenzamides include fluopyram. The furan carboxamides include fenfuram. The oxathiin carboxamides include carboxin and oxycarboxin. The thiazole carboxamides include thifluzamide. The pyrazole-4-carboxamides include benzovindiflupyr, bixafen, flubeneteram (provisional common name, Registry Number 1676101-39-5), fluindapyr, fluxapyroxad, furametpyr, inpyrfluxam, isopyrazam, penflufen, penthiopyrad, pyrapropoyne (provisional common name, Registry Number 1803108-03-3), sedaxane and N-[2-(2,4-dichlorophenyl)-2-methoxy-1-methylethyl]-3-(difluoromethyl)-1-methyl-1H-pyrazole-4-carboxamide. The N-cyclopropyl-N-benzyl-pyrazole carboxamides include isoflucypram. The N-methoxy-(phenyl-ethyl)-pyrazole carboxamides include pydiflumetofen. The pyridine carboxamides include boscalid. The pyrazine carboxamides include pyraziflumid.

“Hydroxy-(2-amino-)pyrimidine fungicides (b8)” (FRAC code 8) inhibit nucleic acid synthesis by interfering with adenosine deaminase. Examples include bupirimate, dimethirimol and ethirimol.

“Anilinopyrimidine (AP) fungicides (b9)” (FRAC code 9) are proposed to inhibit biosynthesis of the amino acid methionine and to disrupt the secretion of hydrolytic enzymes that lyse plant cells during infection. Examples include cyprodinil, mepanipyrim and pyrimethanil.

“N-Phenyl carbamate fungicides (b10)” (FRAC code 10) inhibit mitosis by binding to β-tubulin and disrupting microtubule assembly. Inhibition of microtubule assembly can disrupt cell division, transport within the cell and cell structure. Examples include diethofencarb.

“Quinone outside inhibitor (QoI) fungicides (b11)” (FRAC code 11) inhibit complex III mitochondrial respiration in fungi by affecting ubiquinol oxidase. Oxidation of ubiquinol is blocked at the “quinone outside” (Qo) site of the cytochrome bc₁ complex, which is located in the inner mitochondrial membrane of fungi. Inhibiting mitochondrial respiration prevents normal fungal growth and development. Quinone outside inhibitor fungicides include methoxyacrylate, methoxyacetamide, methoxycarbamate, oximinoacetate, oximinoacetamide and dihydrodioxazine fungicides (collectively also known as strobilurin fungicides), and oxazolidinedione, imidazolinone and benzylcarbamate fungicides. The methoxyacrylates include azoxystrobin, coumoxystrobin, enoxastrobin (also known as enestroburin), flufenoxystrobin, picoxystrobin and pyraoxystrobin. The methoxyacetamides include mandestrobin. The methoxycarbamates include pyraclostrobin, pyrametostrobin and triclopyricarb. The oximinoacetates include kresoxim-methyl and trifloxystrobin. The oximinoacetamides include dimoxystrobin, fenaminstrobin, metominostrobin and orysastrobin. The dihydrodioxazines include fluoxastrobin. The oxazolidinediones include famoxadone. The imidazolinones include fenamidone. The benzylcarbamates include pyribencarb.

“Phenylpyrrole (PP) fungicides (b12)” (FRAC code 12) inhibit a MAP/histidine kinase associated with osmotic signal transduction in fungi. Fenpiclonil and fludioxonil are examples of this fungicide class.

“Azanaphthalene fungicides (b13)” (FRAC code 13) are proposed to inhibit signal transduction by a mechanism which is as yet unknown. They have been shown to interfere with germination and/or appressorium formation in fungi that cause powdery mildew diseases. Azanaphthalene fungicides include aryloxyquinolines and quinazolinones. The aryloxyquinolines include quinoxyfen. The quinazolinones include proquinazid.

“Cell peroxidation inhibitor fungicides (b14)” (FRAC code 14) are proposed to inhibit lipid peroxidation which affects membrane synthesis in fungi. Members of this class, such as etridiazole, may also affect other biological processes such as respiration and melanin biosynthesis. Cell peroxidation fungicides include aromatic hydrocarbon and 1,2,4-thiadiazole fungicides. The aromatic hydrocarboncarbon fungicides include biphenyl, chloroneb, dicloran, quintozene, tecnazene and tolclofos-methyl. The 1,2,4-thiadiazoles include etridiazole.

“Melanin biosynthesis inhibitor-reductase (MBI-R) fungicides (b15)” (FRAC code 16.1) inhibit the naphthal reduction step in melanin biosynthesis. Melanin is required for host plant infection by some fungi. Melanin biosynthesis inhibitor-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

“Melanin biosynthesis inhibitor-dehydratase (MBI-D) fungicides (b16a)” (FRAC code 16.2) inhibit scytalone dehydratase in melanin biosynthesis. Melanin is required for host plant infection by some fungi. Melanin biosynthesis inhibitor-dehydratase fungicides include cyclopropanecarboxamide, carboxamide and propionamide fungicides. The cyclopropanecarboxamides include carpropamid. The carboxamides include diclocymet. The propionamides include fenoxanil.

“Melanin biosynthesis inhibitor-polyketide synthase (MBI-P) fungicides (b16b)” (FRAC code 16.3) inhibit polyketide synthase in melanin biosynthesis. Melanin is required for host plant infection by some fungi. Melanin biosynthesis inhibitor-polyketide synthase fungicides include trifluoroethylcarbamate fungicides. The trifluoroethylcarbamates include tolprocarb.

“Keto reductase inhibitor (KRI) fungicides (b17)” (FRAC code 17) inhibit 3-keto reductase during C4-demethylation in sterol production. Keto reductase inhibitor fungicides (also known as Sterol Biosynthesis Inhibitors (SBI): Class III) include hydroxyanilides and amino-pyrazolinones. Hydroxyanilides include fenhexamid. Amino-pyrazolinones include fenpyrazamine. Quinofumelin (provisional common name, Registry Number 861647-84-9) and ipflufenoquin (provisional common name, Registry Number 1314008-27-9) are also believed to be keto reductase inhibitor fungicides.

“Squalene-epoxidase inhibitor fungicides (b18)” (FRAC code 18) (SBI: Class IV) inhibit squalene-epoxidase in the sterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

“Polyoxin fungicides (b19)” (FRAC code 19) inhibit chitin synthase. Examples include polyoxin.

“Phenylurea fungicides (b20)” (FRAC code 20) are proposed to affect cell division. Examples include pencycuron.

“Quinone inside inhibitor (QiI) fungicides (b21)” (FRAC code 21) inhibit complex III mitochondrial respiration in fungi by affecting ubiquinone reductase. Reduction of ubiquinone is blocked at the “quinone inside” (Qi) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole, sulfamoyltriazole and picolinamide fungicides. The cyanoimidazoles include cyazofamid. The sulfamoyltriazoles include amisulbrom. The picolinamides include fenpicoxamid (Registry Number 517875-34-2).

“Benzamide and thiazole carboxamide fungicides (b22)” (FRAC code 22) inhibit mitosis by binding to β-tubulin and disrupting microtubule assembly. Inhibition of microtubule assembly can disrupt cell division, transport within the cell and cell structure.

The benzamides include toluamides such as zoxamide. The thiazole carboxamides include ethylaminothiazole carboxamides such as ethaboxam.

“Enopyranuronic acid antibiotic fungicides (b23)” (FRAC code 23) inhibit growth of fungi by affecting protein biosynthesis. Examples include blasticidin-S.

“Hexopyranosyl antibiotic fungicides (b24)” (FRAC code 24) inhibit growth of fungi by affecting protein biosynthesis. Examples include kasugamycin.

“Glucopyranosyl antibiotic: protein synthesis fungicides (b25)” (FRAC code 25) inhibit growth of fungi by affecting protein biosynthesis. Examples include streptomycin.

“Glucopyranosyl antibiotic fungicides (b26)” (FRAC code U18, previously FRAC code 26 reclassified to U18) are proposed to inhibit trehalase and inositol biosynthesis. Examples include validamycin.

“Cyanoacetamideoxime fungicides (b27)” (FRAC code 27) include cymoxanil.

“Carbamate fungicides (b28)” (FRAC code 28) are considered multi-site inhibitors of fungal growth. They are proposed to interfere with the synthesis of fatty acids in cell membranes, which then disrupts cell membrane permeability. Iodocarb, propamacarb and prothiocarb are examples of this fungicide class.

“Oxidative phosphorylation uncoupling fungicides (b29)” (FRAC code 29) inhibit fungal respiration by uncoupling oxidative phosphorylation. Inhibiting respiration prevents normal fungal growth and development. This class includes dinitrophenyl crotonates such as binapacryl, meptyldinocap and dinocap, and 2,6-dinitroanilines such as fluazinam.

“Organo tin fungicides (b30)” (FRAC code 30) inhibit adenosine triphosphate (ATP) synthase in oxidative phosphorylation pathway. Examples include fentin acetate, fentin chloride and fentin hydroxide.

“Carboxylic acid fungicides (b31)” (FRAC code 31) inhibit growth of fungi by affecting deoxyribonucleic acid (DNA) topoisomerase type II (gyrase). Examples include oxolinic acid.

“Heteroaromatic fungicides (b32)” (FRAC code 32) are proposed to affect DNA/ribonucleic acid (RNA) synthesis. Heteroaromatic fungicides include isoxazoles and isothiazolones. The isoxazoles include hymexazole and the isothiazolones include octhilinone.

“Phosphonate fungicides (b33)” (FRAC code P07, previously FRAC code 33 reclassified to P07) include phosphorous acid and its various salts, including fosetyl-aluminum.

“Phthalamic acid fungicides (b34)” (FRAC code 34) include teclofthalam.

“Benzotriazine fungicides (b35)” (FRAC code 35) include triazoxide.

“Benzene-sulfonamide fungicides (b36)” (FRAC code 36) include flusulfamide.

“Pyridazinone fungicides (b37)” (FRAC code 37) include diclomezine.

“Thiophene-carboxamide fungicides (b38)” (FRAC code 38) are proposed to affect ATP production. Examples include silthiofam.

“Complex I NADH oxidoreductase inhibitor fungicides (b39)” (FRAC code 39) inhibit electron transport in mitochondria and include pyrimidinamines such as diflumetorim, pyrazole-5-carboxamides such as tolfenpyrad, and quinazoline such as fenazaquin.

“Carboxylic acid amide (CAA) fungicides (b40)” (FRAC code 40) inhibit cellulose synthase which prevents growth and leads to death of the target fungus. Carboxylic acid amide fungicides include cinnamic acid amide, valinamide carbamate and mandelic acid amide fungicides. The cinnamic acid amides include dimethomorph, flumorph and pyrimorph. The valinamide carbamates include benthiavalicarb, benthiavalicarb-isopropyl, iprovalicarb, tolprocarb and valifenalate (also known as valiphenal). The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)-amino]butanamide.

“Tetracycline antibiotic fungicides (b41)” (FRAC code 41) inhibit growth of fungi by affecting protein synthesis. Examples include oxytetracycline.

“Thiocarbamate fungicides (b42)” (FRAC code M12, previously FRAC code 42 reclassified to M12) include methasulfocarb.

“Benzamide fungicides (b43)” (FRAC code 43) inhibit growth of fungi by delocalization of spectrin-like proteins. Examples include pyridinylmethyl benzamides such as fluopicolide and fluopimomide.

“Microbial fungicides (b44)” (FRAC code BM02, previously FRAC code 44 reclassified to BM02) disrupt fungal pathogen cell membranes. Microbial fungicides include Bacillus species such as Bacillus amyloliquefaciens strains AP-136, AP-188, AP-218, AP-219, AP-295, QST713, FZB24, F727, MB1600, D747, TJ100 (also called strain 1 BE; known from EP2962568), and the fungicidal lipopeptides which they produce.

“Quinone outside inhibitor, stigmatellin binding (QoSI) fungicides (b45)” (FRAC code 45) inhibit complex III mitochondrial respiration in fungi by affecting ubiquinone reductase at the “quinone outside” (Qo) site, stigmatellin binding sub-site, of the cytochrome bc₁ complex. Inhibiting mitochondrial respiration prevents normal fungal growth and development. QoSI fungicides include triazolopyrimidylamines such as ametoctradin.

“Plant extract fungicides (b46)” (FRAC code 46) cause cell membrane disruption. Plant extract fungicides include terpene hydrocarbons, terpene alcohols and terpen phenols such as the extract from Melaleuca alternifolia (tea tree) and plant oils (mixtures) such as eugenol, geraniol and thymol.

“Cyanoacrylate fungicides (b47)” (FRAC code 47) bind to the myosin motor domain and effect motor activity and actin assembly. Cyanoacrylates include fungicides such as phenamacril.

“Polyene fungicides (b48)” (FRAC code 48) cause disruption of the fungal cell membrane by binding to ergosterol, the main sterol in the membrane. Examples include natamycin (pimaricin).

“Oxysterol binding protein inhibitor (OSBPI) Fungicides (b49)” (FRAC code 49) bind to the oxysterol-binding protein in oomycetes causing inhibition of zoospore release, zoospore motility and sporangia germination. Oxysterol binding fungicides include piperdinylthiazoleisoxazolines such as oxathiapiprolin and fluoxapiprolin.

“Aryl-phenyl-ketone fungicides (b50)” (FRAC code 50, previously FRAC code U8 reclassified to 50) inhibit the growth of mycelium in fungi. Aryl-phenyl ketone fungicides include benzophenones such as metrafenone, and benzoylpyridines such as pyriofenone.

“Host plant defense induction fungicides (b51)” induce host plant defense mechanisms. Host plant defense induction fungicides include benzothiadiazole (FRAC code P01), benzisothiazole (FRAC code P02), thiadiazole carboxamide (FRAC code P03), polysaccharide (FRAC code P04), plant extract (FRAC code P05), microbial (FRAC code P06) and phosphonate fungicides (FRAC code P07, see (b33) above). The benzothiadiazoles include acibenzolar-S-methyl. The benzisothiazoles include probenazole. The thiadiazole carboxamides include tiadinil and isotianil. The polysaccharides include laminarin. The plant extracts include extract from Reynoutria sachalinensis (giant knotweed). The microbials include Bacillus mycoides isolate J and cell walls of Saccharomyces cerevisiae strain LAS117.

“Multi-site activity fungicides (b52)” inhibit fungal growth through multiple sites of action and have contact/preventive activity. Multi-site activity fungicides include copper fungicides (FRAC code M01), sulfur fungicides (FRAC code M02), dithiocarbamate fungicides (FRAC code M03), phthalimide fungicides (FRAC code M04), chloronitrile fungicides (FRAC code M05), sulfamide fungicides (FRAC code M06), multi-site contact guanidine fungicides (FRAC code M07), triazine fungicides (FRAC code M08), quinone fungicides (FRAC code M09), quinoxaline fungicides (FRAC code M10), maleimide fungicides (FRAC code M11) and thiocarbamate (FRAC code M12, see (b42) above) fungicides. Copper fungicides are inorganic compounds containing copper, typically in the copper(II) oxidation state; examples include copper oxychloride, copper sulfate and copper hydroxide (e.g., including compositions such as Bordeaux mixture (tribasic copper sulfate).

Sulfur fungicides are inorganic chemicals containing rings or chains of sulfur atoms; examples include elemental sulfur. Dithiocarbamate fungicides contain a dithiocarbamate molecular moiety; examples include ferbam, mancozeb, maneb, metiram, propineb, thiram, zinc thiazole, zineb and ziram. Phthalimide fungicides contain a phthalimide molecular moiety; examples include folpet, captan and captafol. Chloronitrile fungicides contain an aromatic ring substituted with chloro and cyano; examples include chlorothalonil. Sulfamide fungicides include dichlofluanid and tolyfluanid. Multi-site contact guanidine fungicides include, guazatine, iminoctadine albesilate and iminoctadine triacetate. Triazine fungicides include anilazine. Quinone fungicides include dithianon. Quinoxaline fungicides include quinomethionate (also known as chinomethionate). Maleimide fungicides include fluoroimide.

“Biologicals with multiple modes of action (b53)” include agents from biological origins showing multiple mechanisms of action without evidence of a dominating mode of action. This class of fungicides includes polypeptide (lectin), phenol, sesquiterpene, tritepenoid and coumarin fungicides (FRAC code BM01) such as extract from the cotyledons of lupine plantlets. This class also includes momicrobial fungicides (FRAC code BM02, see (b44) above).

“Fungicides other than fungicides of component (a) and components (b1) through (b53); (b54)”; include certain fungicides whose mode of action may be unknown. These include: (b54.1) “phenyl-acetamide fungicides” (FRAC code U06), (b54.2) “guanidine fungicides” (FRAC code U12), (b54.3) “thiazolidine fungicides” (FRAC code U13), (b54.4) “pyrimidinone-hydrazone fungicides” (FRAC code U14), (b54.5) “4-quinolylacetate fungicides” (FRAC code U16), (54.6) “tetrazolyloxime fungicides” (FRAC code U17) and “glucopyranosyl antibiotic fungicides” (FRAC code U18, see (b26) above). The phenyl-acetamides include cyflufenamid. The guanidines include dodine. The thiazolidines include flutianil. The pyrimidinonehydrazones include ferimzone. The 4-quinolylacetates include tebufloquin. The tetrazolyloximes include picarbutrazox.

The (b54) class also includes bethoxazin, dichlobentiazox (provisional common name, Registry Number 957144-77-3), dipymetitrone (provisional common name, Registry Number 16114-35-5), flometoquin, neo-asozin (ferric methanearsonate), pyrrolnitrin, tolnifanide (Registry Number 304911-98-6), N-[4-[4-chloro-3-(trifluoromethyl)phenoxy]-2,5-dimethylphenyl]-N-ethyl-N-methylmethanimidamide, 5-fluoro-2-[(4-fluoro-phenyl)methoxy]-4-pyrimidinamine and 4-fluorophenyl N-[1-[[[1-(4-cyanophenyl)ethyl]-sulfonyl]methyl]propyl]carbamate.

Additional “Fungicides other than fungicides of classes (1) through (54)” whose mode of action may be unknown, or may not yet be classified include a fungicidal compound selected from components (b54.7) through (b54.12), as described below.

Component (54.7) relates to (1S)-2,2-bis(4-fluorophenyl)-1-methylethyl N-[[3-(acetyloxy)-4-methoxy-2-pyridinyl]carbonyl]-L-alaninate (provisional common name florylpicoxamid, Registry Number 1961312-55-9) which is believed to be a Quinone inside inhibitor (QiI) fungicide (FRAC code 21) inhibiting the Complex III mitochondrial respiration in fungi.

Component (54.8) relates to 1-[2-[[[1-(4-chlorophenyl)-1H-pyrazol-3-yl]oxy]methyl]-3-methylphenyl]-1,4-dihydro-4-methyl-5H-tetrazol-5-one (provisional common name metyltetraprole, Registry Number 1472649-01-6), which is believed to be a quinone outside inhibitor (QoI) fungicide (FRAC code 45) inhibiting the Complex III mitochondrial respiration in fungi, and is effective against QoI resistant strains.

Component (54.9) relates to 3-chloro-4-(2,6-difluorophenyl)-6-methyl-5-phenylpyridazine (provisional common name pyridachlometyl, Registry Number 1358061-55-8), which is believed to be promoter tubulin polymerization, resulting antifungal activity against fungal species belonging to the phyla Ascomycota and Basidiomycota.

Component (54.10) relates to (4-phenoxyphenyl)methyl 2-amino-6-methyl-pyridine-3-carboxylate (provisional common name aminopyrifen, Registry Number 1531626-08-0) which is believed to inhibit GWT-1 protein in glycosylphosphatidylinositol-anchor biosynthesis in Neurospora crassa.

Component (b54.11) relates to a compound of Formula b54.11

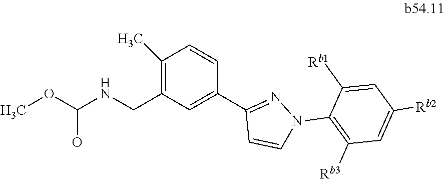

wherein

R^{b1} and R^{b3} are each independently halogen; and

R^{b2} is H, halogen, C₁-C₃ alkyl, C₁-C₃ haloalkyl or C₃-C₆ cycloalkyl.

Examples of compounds of Formula b54.11 include (b54.11a) methyl N-[[5-[1-(2,6-difluoro-4-formylphenyl)-1H-pyrazol-3-yl]-2-methylphenyl] methyl]carbamate, (b54.11b) methyl N-[[5-[1-(4-cyclopropyl-2,6-dichlorophenyl)-1H-pyrazol-3-yl]-2-methylphenyl]-methyl]carbamate, (b54.11c) methyl N-[[5-[1-(4-chloro-2,6-difluorophenyl)-1H-pyrazol-3-yl]-2-methylphenyl]methyl]carbamate, (b54.11d) methyl N-[[5-[1-(4-cyclopropyl-2,6-difluorophenyl)-1H-pyrazol-3-yl]-2-methylphenyl]methyl]carbamate, (b54.11e) methyl N-[[5-[1-[2,6-difluoro-4-(1-methylethyl)phenyl]-1H-pyrazol-3-yl]-2-methylphenyl]methyl]carbamate and (b54.11f) methyl N-[[5-[1-[2,6-difluoro-4-(trifluoromethyl)phenyl]-1H-pyrazol-3-yl]-2-methylphenyl]methyl]carbamate. Compounds of Formula b54.11, their use as fungicides and methods of preparation are generally known; see, for example, PCT Patent Publications WO 2008/124092, WO 2014/066120 and WO 2020/097012. methyl

Component (b54.12) relates to a compound of Formula b54.12

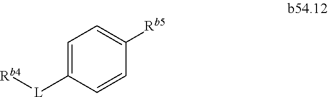

wherein
R^{b4} is

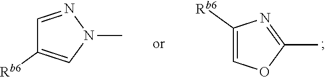

R^{b6} is C₂-C₄ alkoxycarbonyl or C₂-C₄ haloalkylaminocarbonyl;
L is CH₂ or CH₂O, wherein the atom to the right is connected to the phenyl ring in Formula b54.12;
R^{b5} is

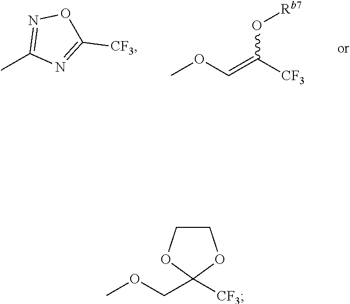

and
R^{b7} is C₁-C₃ alkyl, wherein the wavy bond indicates the adjacent double bond is either (Z)- or (E)-configuration, or a mixture thereof.
   Examples of compounds of Formula b54.12 include (b54.12a)N-(2,2,2-trifluoroethyl)-2-[[4-[5-(trifluoromethyl)-1,2,4-oxadiazol-3-yl]phenyl]methyl]-4-oxazolecarboxamide, (b54.12b) ethyl 1-[[4-[5-(trifluoromethyl)-1,2,4-oxadiazol-3-yl]phenoxy]methyl]-1H-pyrazole-4-carboxylate, (b54.12c) ethyl 1-[[4-[[(1Z)-2-ethoxy-3,3,3-trifluoro-1-propen-1-yl]oxy]phenyl]methyl]-1H-pyrazole-4-carboxylate and (b54.12d) ethyl 1-[[4-[[2-(trifluoromethyl)-1,3-dioxolan-2-yl]methoxy]phenyl]methyl]-1H-pyrazole-4-carboxylate. Compounds of Formula b54.12, their use as fungicides and methods of preparation are generally known; see, for example, PCT Patent Publications WO 2008/187553 and WO 2020/056090.

Embodiments of the present invention as described in the Summary of the Invention include those described below. In the following Embodiments, Formula 1 includes stereoisomers, N-oxides, and salts thereof, and reference to “a compound of Formula 1” includes the definitions of substituents specified in the Summary of the Invention unless further defined in the Embodiments.
Embodiment 1. The composition comprising components (a) and (b) described in the Summary of the Invention wherein in Formula 1, wherein R¹ is methyl.
Embodiment 2. The composition comprising components (a) and (b) described in the Summary of the Invention wherein in Formula 1, wherein R¹ is ethyl.
Embodiment 3. The composition comprising components (a) and (b) described in the Summary of the Invention wherein in Formula 1, or Embodiments 1 or 2, wherein R² is cyano, halogen or C₁-C₂ alkyl.
Embodiment 4. The composition comprising components (a) and (b) described in the Summary of the Invention wherein in Formula 1, or Embodiments 1 or 2, wherein R² is cyano, Br, Cl, F, C₁-C₂ alkyl or C₁-C₂ haloalkyl.
Embodiment 5. The composition of Embodiment 4 wherein R² is cyano, Br, Cl, F, C₁-C₂ alkyl or halomethyl.
Embodiment 6. The composition of Embodiment 5 wherein R² is cyano, Br, Cl, F, C₁-C₂ alkyl or CF₃.
Embodiment 7. The composition of Embodiment 6 wherein R² is cyano, Br, Cl, F or C₁-C₂ alkyl.
Embodiment 8. The composition of Embodiment 7 wherein R² is cyano or C₁-C₂ alkyl.
Embodiment 9. The composition of Embodiment 8 wherein R² is C₁-C₂ alkyl.
Embodiment 10. The composition of Embodiment 8 wherein R² is cyano or methyl.
Embodiment 11. The composition of Embodiment 10 wherein R² is methyl.
Embodiment 12. The composition of Embodiment 7 wherein R² is Br, Cl or methyl.
Embodiment 13. The composition comprising components (a) and (b) described in the Summary of the Invention wherein in Formula 1, or any one of Embodiments 1 through 12, wherein R³ is halogen or methyl.
Embodiment 13a. The composition of Embodiment 13 wherein R³ is halogen.
Embodiment 13b. The composition of Embodiment 13 wherein R³ is Br, Cl, F or methyl.
Embodiment 14. The composition of Embodiment 13 wherein R³ is Br, Cl or F.
Embodiment 15. The composition of Embodiment 14 wherein R³ is Cl or F.
Embodiment 16. The composition of Embodiment 15 wherein R³ is Cl.
Embodiment 17. The composition of Embodiment 15 wherein R³ is F.
Embodiment 18. The composition of Embodiment 13 wherein R³ is Cl, F or methyl.
Embodiment 19. The composition of Embodiment 18 wherein R³ is Cl or methyl.
Embodiment 20. The composition of Embodiment 19 wherein R³ is methyl.
Embodiment 21. The composition comprising components (a) and (b) described in the Summary of the Invention wherein in Formula 1, or any one of Embodiments 1 through 20, wherein each R⁴ is independently halogen, cyano, C₁-C₂ alkyl, C₁-C₂ alkoxy, C₁-C₂ haloalkoxy, C₂-C₄ alkenyloxy, C₂-C₄ alkynyloxy, C₂-C₄ cyanoalkoxy, C₂-C₄ alkoxyalkyl or C₂-C₄ alkoxyalkoxy.
Embodiment 22. The composition of Embodiment 21 wherein each R⁴ is independently halogen, cyano, methyl, methoxy, halomethoxy, C₂-C₄ alkenyloxy, C₂-C₄ alkynyloxy, C₂-C₄ cyanoalkoxy, C₂-C₄ alkoxyalkyl or C₂-C₄ alkoxyalkoxy.
Embodiment 23. The composition of Embodiment 22 wherein each R⁴ is independently halogen, cyano, methyl, methoxy, halomethoxy, C₂-C₄ alkenyloxy, C₂-C₄ alkynyloxy or C₂-C₄ cyanoalkoxy.
Embodiment 24. The composition of Embodiment 23 wherein each R⁴ is independently halogen, cyano, methyl, methoxy, halomethoxy or C₂-C₄ cyanoalkoxy.
Embodiment 25. The composition of Embodiment 24 wherein each R⁴ is independently halogen, cyano, methyl or methoxy.
Embodiment 25a. The composition of Embodiment 25 wherein each R⁴ is independently halogen, cyano or methoxy.
Embodiment 25b. The composition of Embodiment 25 wherein each R⁴ is independently halogen, cyano or methyl.
Embodiment 26. The composition of Embodiment 25 wherein each R⁴ is independently Br, Cl, F, cyano, methyl or methoxy.
Embodiment 27. The composition of Embodiment 26 wherein each R⁴ is independently Br, Cl, F, cyano or methoxy.
Embodiment 28. The composition of Embodiment 27 wherein each R⁴ is independently Cl, F, cyano or methoxy.
Embodiment 29. The composition of Embodiment 27 wherein each R⁴ is independently Br, Cl or F.
Embodiment 30. The composition of Embodiment 29 wherein each R⁴ is independently Cl or F.
Embodiment 31. The composition of Embodiment 30 wherein each R⁴ is Cl Embodiment 32. The composition of Embodiment 30 wherein each R⁴ is F.
Embodiment 33. The composition comprising components (a) and (b) described in the Summary of the Invention wherein in Formula 1, or any one of Embodiments 1 through 32, wherein each R⁴ is independently halogen, cyano or C₁-C₂ alkoxy.
Embodiment 34. The composition of Embodiment 33 wherein each R⁴ is independently halogen.
Embodiment 35. The composition of Embodiment 33 wherein each R⁴ is independently Br, Cl or F or cyano.
Embodiment 36. The composition comprising components (a) and (b) described in the Summary of the Invention wherein in Formula 1, or any one of Embodiments 1 through 35, wherein m is 0, 1 or 2.
Embodiment 37. The composition of Embodiment 36 wherein m is 1 or 2.
Embodiment 38. The composition of Embodiment 37 wherein m is 1.
Embodiment 39. The composition of Embodiment 38 wherein m is 2.
Embodiment 40. The composition comprising components (a) and (b) described in the Summary of the Invention wherein in Formula 1, or any one of Embodiments 1 through 39, wherein each R⁵ is independently halogen, C₁-C₂ alkyl, C₂-C₄ alkoxyalkyl, C₁-C₂ alkoxy, C₁-C₂ haloalkoxy, C₂-C₄ alkenyloxy, C₂-C₄ alkynyloxy, C₂-C₄ cyanoalkoxy or C₂-C₄ alkoxyalkoxy.
Embodiment 41. The composition of Embodiment 40 wherein each R⁵ is independently halogen, methyl, methoxy, halomethoxy, C₂-C₄ alkenyloxy, C₂-C₄ alkynyloxy or C₂-C₄ cyanoalkoxy.
Embodiment 42. The composition of Embodiment 41 wherein each R⁵ is independently halogen, methyl, methoxy, halomethoxy, C₂-C₄ alkenyloxy or C₂-C₄ cyanoalkoxy.
Embodiment 43. The composition of Embodiment 42 wherein each R⁵ is independently halogen, methyl, methoxy, halomethoxy or C₂-C₄ cyanoalkoxy.
Embodiment 44. The composition of Embodiment 43 wherein each R⁵ is independently halogen, methyl or methoxy.
Embodiment 45. The composition of Embodiment 44 wherein each R⁵ is independently Br, Cl, F, methyl or methoxy.
Embodiment 46. The composition of Embodiment 45 wherein each R⁵ is independently Br, Cl, F or methoxy.
Embodiment 46a. The composition of Embodiment 46 wherein each R⁵ is independently Br, Cl or F.
Embodiment 47. The composition of Embodiment 46 wherein each R⁵ is independently Cl, F or methoxy.
Embodiment 48. The composition of Embodiment 47 wherein each R⁵ is independently Cl or F.
Embodiment 49. The composition of Embodiment 45 wherein each R⁵ is independently Br, Cl, F or methyl.
Embodiment 50. The composition of Embodiment 49 wherein each R⁵ is independently F or methyl.
Embodiment 51. The composition of Embodiment 50 wherein each R⁵ is F.
Embodiment 52. The composition of Formula 1 or any one of Embodiments 1 through 51 wherein n is 0, 1 or 2.
Embodiment 53. The composition of Embodiment 52 wherein n is 1 or 2.
Embodiment 54. The composition of Embodiment 53 wherein n is 1.
Embodiment 55. The composition of Embodiment 53 wherein n is 2.
Embodiment 56. The composition comprising components (a) and (b) described in the Summary of the Invention wherein in Formula 1, or any one of Embodiments 1 through 55, wherein R⁶ is H; or C₁-C₃ alkyl or C₁-C₃ haloalkyl, each optionally substituted with up to 1 substituent selected from R^{6a}; or amino, C₂-C₃ alkenyl, C₂-C₃ alkynyl, cyclopropyl, CH(═O), S(═O)₂OM, S(═O)ᵤR⁷, (C═W)R⁸ or OR⁹.
Embodiment 57. The composition of Embodiment 56 wherein R⁶ is H; or C₁-C₃ alkyl or C₁-C₃ haloalkyl, each optionally substituted with up to 1 substituent selected from R^{6a}; or cyclopropyl, S(═O)₂OM, S(═O)ᵤR⁷, (C═W)R⁸ or OR⁹.
Embodiment 58. The composition of Embodiment 57 wherein R⁶ is H; or C₁-C₂ alkyl or C₁-C₂ haloalkyl, each optionally substituted with up to 1 substituent selected from R^{6a}; or S(═O)ᵤR⁷ or OR⁹.
Embodiment 59. The composition of Embodiment 58 wherein R⁶ is H; or C₁-C₂ alkyl or C₁-C₂ haloalkyl, each optionally substituted with up to 1 substituent selected from R^{6a}.
Embodiment 60. The composition of Embodiment 59 wherein R⁶ is H, C₁-C₂ alkyl or C₁-C₂ haloalkyl.
Embodiment 61. The composition of Embodiment 60 wherein R⁶ is H, methyl or halomethyl.
Embodiment 62. The composition of Embodiment 61 wherein R⁶ is H, methyl or trifluoromethyl.
Embodiment 63. The composition of Embodiment 62 wherein R⁶ is H or methyl.
Embodiment 64. The composition of Embodiment 63 wherein R⁶ is H.
Embodiment 65. The composition of Formula 1 or any one of Embodiments 1 through 64 wherein each R^{6a} is independently cyano, C₃-C₆ cycloalkyl or C₁-C₃ alkoxy.
Embodiment 66. The composition of Embodiment 65 wherein each R^{6a} is independently cyano, cyclopropyl or methoxy.
Embodiment 67. The composition of Embodiment 66 wherein each R^{6a} is independently cyano or cyclopropyl.
Embodiment 68. The composition comprising components (a) and (b) described in the Summary of the Invention wherein in Formula 1, or any one of Embodiments 1 through 58, wherein u is 0.
Embodiment 69. The composition comprising components (a) and (b) described in the Summary of the Invention wherein in Formula 1, or any one of Embodiments 1 through 58, wherein R⁷ is methyl or halomethyl.
Embodiment 70. The composition comprising components (a) and (b) described in the Summary of the Invention wherein in Formula 1, or any one of Embodiments 1 through 57, wherein W is 0.
Embodiment 71. The composition comprising components (a) and (b) described in the Summary of the Invention wherein in Formula 1, or any one of Embodiments 1 through 57, wherein R⁸ is C₁-C₃ alkyl, C₁-C₃ alkoxy or C₁-C₃ alkylthio.
Embodiment 72. The composition of Embodiment 71 wherein R⁸ is methyl, ethyl, methoxy, ethoxy, methylthio or ethylthio.
Embodiment 73. The composition of Embodiment 72 wherein R⁸ is methyl, methoxy or methylthio.
Embodiment 74. The composition comprising components (a) and (b) described in the Summary of the Invention wherein in Formula 1, or any one of Embodiments 1 through 58, wherein R⁹ is H; or C₁-C₃ alkyl or C₁-C₃ haloalkyl, each optionally substituted with up to 1 substituent selected from R^{9a}; or CH(═O), cyclopropyl, S(═O)₂OM or (C═W)R¹⁰.
Embodiment 75. The composition of Embodiment 74 wherein R⁹ is H; or C₁-C₂ alkyl or C₁-C₂ haloalkyl, each optionally substituted with up to 1 substituent selected from R^{9a}.
Embodiment 76. The composition comprising components (a) and (b) described in the Summary of the Invention wherein in Formula 1, or any one of Embodiments 1 through 75, wherein each R^{9a} is independently cyano, C₃-C₆ cycloalkyl or C₁-C₃ alkoxy.
Embodiment 77. The composition of Embodiment 76 wherein each R^{9a} is independently cyano, cyclopropyl or methoxy.
Embodiment 78. The composition of Embodiment 77 wherein each R^{9a} is independently cyano or cyclopropyl.
Embodiment 79. The composition comprising components (a) and (b) described in the Summary of the Invention wherein in Formula 1, or any one of Embodiments 1 through 78, wherein R¹⁰ is C₁-C₃ alkyl, C₁-C₃ alkoxy or C₁-C₃ alkylthio.
Embodiment 80. The composition of Embodiment 79 wherein R¹⁰ is methyl, ethyl, methoxy, ethoxy, methylthio or ethylthio.
Embodiment 81. The composition of Embodiment 80 wherein R¹⁰ is methyl, methoxy or methylthio.
Embodiment 82. The composition comprising components (a) and (b) described in the Summary of the Invention wherein in Formula 1, or any one of Embodiments 1 through 81, wherein m is 1 and R⁴ is at the 4-position (or para position), relative to the connection of the phenyl ring to the remainder of Formula 1.
Embodiment 83. The composition comprising components (a) and (b) described in the Summary of the Invention wherein in Formula 1, or any one of Embodiments 1 through 81, wherein m is 1 and R⁴ is at the 6-position (or ortho position), relative to the connection of the phenyl ring to the remainder of Formula 1.
Embodiment 84. The composition comprising components (a) and (b) described in the Summary of the Invention wherein in Formula 1, or any one of Embodiments 1 through 81, wherein m is 1 and R⁴ is at the 4-position (or para position); or m is 1 and R⁴ is at the 6-position (or ortho position), relative to the connection of the phenyl ring to the remainder of Formula 1.
Embodiment 85. The composition comprising components (a) and (b) described in the Summary of the Invention wherein in Formula 1, or any one of Embodiments 1 through 81, wherein m is 2 and one R⁴ is at the 4-position (or para position), and the other is at the 6-position (or ortho position), relative to the connection of the phenyl ring to the remainder of Formula 1).
Embodiment 86. The composition comprising components (a) and (b) described in the Summary of the Invention wherein in Formula 1, or any one of Embodiments 1 through 81, wherein m is 1 and R⁴ is at the 4-position (or para position); or m is 1 and R⁴ is at the 6-position (or ortho position); or m is 2 and one R⁴ is at the 4-position (or para position), and the other is at the 6-position (or ortho position), relative to the connection of the phenyl ring to the remainder of Formula 1.
Embodiment 86a. The composition of Embodiment 86 wherein m is 1 and R⁴ is at the 4-position (or para position); or m is 2 and one R⁴ is at the 4-position (or para position), and the other is at the 6-position (or ortho position), relative to the connection of the phenyl ring to the remainder of Formula 1.
Embodiment 87. The composition comprising components (a) and (b) described in the Summary of the Invention wherein in Formula 1, or any one of Embodiments 1 through 86a, wherein n is 1 and R⁵ is at the 4-position (or the para position), relative to the connection of the nitoanilino ring to the remainder of Formula 1.
Embodiment 88. The composition comprising components (a) and (b) described in the Summary of the Invention wherein in Formula 1, or any one of Embodiments 1 through 86a, wherein n is 1 and R⁵ is at the 6-position (or ortho position), relative to the connection of the nitoanilino ring to the remainder of Formula 1.
Embodiment 89. The composition comprising components (a) and (b) described in the Summary of the Invention wherein in Formula 1, or any one of Embodiments 1 through 86a, wherein n is 2 and one R⁵ is at the 4-position (or para position), and the other is at the 6-position (or ortho position), relative to the connection of the nitoanilino ring to the remainder of Formula 1.
Embodiment 90. The composition comprising components (a) and (b) described in the Summary of the Invention wherein in Formula 1, or any one of Embodiments 1 through 86a, wherein n is 1 and R⁵ is at the 4-position (or para position); or n is 1 and R⁵ is at the 6-position (or ortho position); or n is 2 and one R⁵ is at the 4-position (or para position), and the other is at the 6-position (or ortho position), relative to the connection of the nitoanilino ring to the remainder of Formula 1.
Embodiment 91. The composition comprising components (a) and (b) described in the Summary of the Invention wherein in Formula 1, or any one of Embodiments 1 through 90, wherein m and n are each 1 and R⁴ is at the 4-position (or para position), and R⁵ is at the 6-position (or ortho position); or m is 1 and R⁴ is at the 4-position (or para position), and n is 2 and one R⁵ is at the 4-position (or para position) and the other is at the 6-position (or ortho position); or m and n are each 1 and R⁴ is at the 4-position (or para position), and R⁵ is at the 4-position (or para position); or m is 2 and one R⁴ is at the 4-position (or para position) and the other is at the 6-position (or ortho position), and n is 1 and R⁵ is at the 6-position (or ortho position), relative to the connection of the phenyl and nitoanilino rings to the remainder of Formula 1.
Embodiment 92. The composition of Embodiment 91 wherein m and n are each 1 and R⁴ is at the 4-position (or para position) and R⁵ is at the 6-position (or ortho position); or m is 1 and R⁴ is at the 4-position (or para position), and n is 2 and one R⁵ is at the 4-position (or para position) and the other is at the 6-position (or ortho position); m is 2 and one R⁴ is at the 4-position (or para position) and the other is at the 6-position (or ortho position), and n is 1 and R⁵ is at the 6-position (or ortho position), relative to the connection of the phenyl and nitoanilino rings to the remainder of Formula 1.
Embodiment 93. The composition of Embodiment 92 wherein m and n are each 1 and R⁴ is at the 4-position (or para position) and R⁵ is at the 6-position (or ortho position); or m is 1 and R⁴ is at the 4-position (or para position), and n is 2 and one R⁵ is at the 4-position (or para position) and the other is at the 6-position (or ortho position), relative to the connection of the phenyl and nitoanilino rings to the remainder of Formula 1.
Embodiment 94. The composition of Embodiment 93 wherein m and n are each 1 and R⁴ is at the 4-position (or para position) and R⁵ is at the 6-position (or ortho position).
Embodiment 95. The composition of Embodiment 93 wherein m is 1 and R⁴ is at the 4-position (or para position), and n is 2 and one R⁵ is at the 4-position (or para position) and the other is at the 6-position (or ortho position).
Embodiment 96. The composition comprising components (a) and (b) described in the Summary of the Invention or any one of Embodiments 1 through 95 wherein component (a) does not comprise an N-oxide of a compound of Formula 1.
Embodiment 97. The composition comprising components (a) and (b) described in the Summary of the Invention or any one of Embodiments 1 through 95 wherein component (a) comprises a compound selected from the group consisting of
4-(2-bromo-4,6-difluorophenyl)-N-(2-fluoro-6-nitrophenyl)-1,3-dimethyl-1H-pyrazol-5-amine (Compound 1),
3-chloro-4-[5-[(2-chloro-4-fluoro-6-nitrophenyl)amino]-1,3-dimethyl-1H-pyrazol-4-yl]benzonitrile (Compound 18),
N-(2-chloro-4-fluoro-6-nitrophenyl)-4-(2-chloro-4-fluorophenyl)-1,3-dimethyl-1H-pyrazol-5-amine (Compound 19),
4-(2-chloro-6-fluorophenyl)-N-(2-fluoro-4-methoxy-6-nitrophenyl)-1,3-dimethyl-1H-pyrazol-5-amine (Compound 23),
4-(2,4-difluorophenyl)-N-(2-fluoro-4-methoxy-6-nitrophenyl)-1,3-dimethyl-1H-pyrazol-5-amine (Compound 57),
4-(2-bromo-4-fluorophenyl)-N-(2-fluoro-6-nitrophenyl)-1,3-dimethyl-1H-pyrazol-5-amine (Compound 60),
4-(2-chloro-4,6-difluorophenyl)-N-(2-fluoro-6-nitrophenyl)-1,3-dimethyl-1H-pyrazol-5-amine (Compound 68),
4-(2-chloro-4-fluorophenyl)-N-(2-fluoro-6-nitrophenyl)-3-ethyl-1-methyl-1H-pyrazol-5-amine (Compound 72),
N-(2-chloro-4-fluoro-6-nitrophenyl)-4-(2-chloro-4-methoxyphenyl)-1,3-dimethyl-1H-pyrazol-5-amine (Compound 73),
4-(2-chloro-4-fluorophenyl)-N-(2-fluoro-4-methyl-6-nitrophenyl)-1,3-dimethyl-1H-pyrazol-5-amine (Compound 93),
4-(2-chloro-4-fluorophenyl)-N-(4-fluoro-2-nitrophenyl)-1,3-dimethyl-1H-pyrazol-5-amine (Compound 111),
4-(2-chloro-4-fluorophenyl)-N-(2-fluoro-6-nitrophenyl)-1,3-dimethyl-1H-pyrazol-5-amine (Compound 112),
4-(2,4-difluorophenyl)-N-(2-fluoro-6-nitrophenyl)-1,3-dimethyl-1H-pyrazol-5-amine (Compound 118),
N-(4-chloro-2-fluoro-6-nitrophenyl)-4-(2-chloro-4-fluorophenyl)-1,3-dimethyl-1H-pyrazol-5-amine (Compound 121) and
3-chloro-4-[5-[(2-fluoro-4-methyl-6-nitrophenyl)amino]-1,3-dimethyl-1H-pyrazol-4-yl]benzonitrile (Compound 127).
   Embodiment 98. The composition of Embodiment 97 wherein component (a) comprises a compound selected from the group consisting of Compounds 1, 19, 57, 60, 68, 72, 93, 112, 121 and 127.
   Embodiment 99. The composition of Embodiment 98 wherein component (a) comprises a compound selected from the group consisting of Compounds 68, 72 and 112.
   Embodiment 100. The composition of Embodiment 99 wherein component (a) comprises Compound 68.
   Embodiment 101. The composition of Embodiment 99 wherein component (a) comprises Compound 72.
   Embodiment 102. The composition of Embodiment 99 wherein component (a) comprises Compound 112.
   Embodiment 103. The composition comprising components (a) and (b) described in the Summary of the Invention or any one of Embodiments 1 through 99 wherein component (a) is 4-(2-chloro-4,6-difluorophenyl)-N-(2-fluoro-6-nitrophenyl)-1,3-dimethyl-1H-pyrazol-5-amine.
   Embodiment 104. The composition comprising components (a) and (b) described in the Summary of the Invention or any one of Embodiments 1 through 99 wherein component (a) is 4-(2-chloro-4-fluorophenyl)-N-(2-fluoro-6-nitrophenyl)-3-ethyl-1-methyl-1H-pyrazol-5-amine.
   Embodiment 105. The composition comprising components (a) and (b) described in the Summary of the Invention or any one of Embodiments 1 through 99 wherein component (a) is 4-(2-chloro-4-fluorophenyl)-N-(2-fluoro-6-nitrophenyl)-1,3-dimethyl-1H-pyrazol-5-amine.
   Embodiment 106. The composition of Embodiments 1 through 105 wherein component (b) comprises at least two fungicidal compounds, and when component (b) consists of a binary combination of two fungicidal compounds, wherein one of the fungicidal compounds is cyproconazole, difenconazole, epoxiconazole, flutriafol, metconazole, prothioconazole or tebuconazole then the other fungicidal compound is other than azoxystrobin, benzovindiflupyr, bixafen, boscalid, fluopyram, luindapyr, fluxapyroxad, isopyrazam, kresoxim-methyl, penthiopyrad, picoxystrobin, proquinazid, pyraclostrobin, quinoxyfen, sedaxane or trifloxystrobin.
   Embodiment 107. The composition of Embodiment 106 wherein component (b) comprises at least two fungicidal compounds, and when component (b) consists of a binary combination of two fungicidal compounds, wherein one of the fungicidal compounds is cyproconazole, difenconazole, epoxiconazole, flutriafol, prothioconazole or tebuconazole then the other fungicidal compound is other than azoxystrobin, benzovindiflupyr, bixafen, fluindapyr, fluxapyroxad, isopyrazam, picoxystrobin, pyraclostrobin or trifloxystrobin.
   Embodiment 108. The composition of Embodiment 107 wherein (b) comprises at least two fungicidal compounds, and when component (b) consists of a binary combination of two fungicidal compounds, wherein one of the fungicidal compounds is cyproconazole, difenconazole, epoxiconazole, flutriafol, prothioconazole or tebuconazole then the other fungicidal compound is other than azoxystrobin, benzovindiflupyr, bixafen, fluindapyr, fluxapyroxad, picoxystrobin, pyraclostrobin or trifloxystrobin.

Embodiments of this invention, including Embodiments 1-108 above as well as any other embodiments described herein, can be combined in any manner, and the descriptions of variables in the embodiments not only to the compositions comprising compounds of Formula 1 with at least one other fungicidal compound but also to compositions comprising compounds of Formula 1 with at least one invertebrate pest control compound or agent, and also to the compounds of Formula 1 and their compositions, and also to the starting compounds and intermediate compounds useful for preparing the compounds of Formula 1. In addition, embodiments of this invention, including Embodiments 1-108 above as well as any other embodiments described herein, and any combination thereof, pertain to the methods of the present invention. Therefore, of note as a further embodiment is the composition disclosed above comprising (a) at least one compound selected from the compounds of Formula 1 described above, N-oxides, and salts thereof; and at least one invertebrate pest control compound or agent.

Combinations of Embodiments 1-108 are illustrated by:
Embodiment A. The composition comprising components (a) and (b) described Summary of the Invention wherein component (a) comprises a compound of Formula 1 or salt thereof, wherein in Formula 1,
   R¹ is methyl;
   R² is cyano, halogen or C₁-C₂ alkyl;
   R³ is halogen;
   each R⁴ is independently halogen, cyano, methyl, methoxy, halomethoxy, C₂-C₄ alkenyloxy, C₂-C₄ alkynyloxy or C₂-C₄ cyanoalkoxy;
   each R⁵ is independently halogen, methyl, methoxy, halomethoxy, C₂-C₄ alkenyloxy, C₂-C₄ alkynyloxy or C₂-C₄ cyanoalkoxy;
   R⁶ is H; or C₁-C₂ alkyl or C₁-C₂ haloalkyl, each optionally substituted with up to 1 substituent selected from R^{6a}; or S(═O)ᵤR⁷ or OR⁹;
   R^{6a} is cyano, C₃-C₆ cycloalkyl or C₁-C₃ alkoxy;
   R⁷ is methyl or halomethyl;
   R⁹ is H; or C₁-C₂ alkyl or C₁-C₂ haloalkyl, each optionally substituted with up to 1 substituent selected from R^{9a}; and
   R^{9a} is cyano, C₃-C₆ cycloalkyl or C₁-C₃ alkoxy.
Embodiment B. The composition of Embodiment B wherein in Formula 1,
   R² is methyl or ethyl;
   R³ is Br, Cl or F;
   each R⁴ is independently halogen, cyano, methyl or methoxy;
   m is 1 and R⁴ is at the 4-position (or para position); or m is 1 and R⁴ is at the 6-position (or ortho position); or m is 2 and one R⁴ is at the 4-position (or para position), and the other is at the 6-position (or ortho position); each R⁵ is independently halogen, methyl or methoxy;
   n is 1 and R⁵ is at the 4-position (or para position); or n is 1 and R⁵ is at the 6-position (or ortho position); or n is 2 and one R⁵ is at the 4-position (or para position), and the other is at the 6-position (or ortho position); and
   R⁶ is H or methyl.
Embodiment C. The composition of Embodiment B wherein in Formula 1,
   R² is methyl;
   each R⁴ is independently Br, Cl, F, cyano or methoxy;
   each R⁵ is independently Br, Cl, F, methyl or methoxy; and
   R⁶ is H.
Embodiment D. The composition of Embodiment C wherein in Formula 1,
   each R⁴ is independently Br, Cl or F;
   each R⁵ is independently Br, Cl, F or methoxy; and
   m and n are each 1 and R⁴ is at the 4-position (or para position) and R⁵ is at the 6-position (or ortho position); or m is 1 and R⁴ is at the 4-position (or para position), and n is 2 and one R⁵ is at the 4-position (or para position) and the other is at the 6-position (or ortho position); or m is 2 and one R⁴ is at the 4-position (or para position) and the other is at the 6-position (or ortho position), and n is 1 and R⁵ is at the 6-position (or ortho position).
Embodiment E. The composition of Embodiment D wherein in Formula 1,
   R⁴ is Cl or F;
   each R⁵ is independently Cl, F or methoxy; and m and n are each 1 and R⁴ is at the 4-position (or para position) and R⁵ is at the 6-position (or ortho position); or m is 1 and R⁴ is at the 4-position (or para position), and n is 2 and one R⁵ is at the 4-position (or para position) and the other is at the 6-position (or ortho position).
Embodiment F. The composition comprising components (a) and (b) described Summary of the Invention wherein component (a) comprises a compound of Formula 1 or salt thereof, wherein in Formula 1,
   R¹ is C₁-C₂ alkyl;
   R² is cyano, halogen, C₁-C₂ alkyl or C₁-C₂ haloalkyl;
   R³ is halogen or methyl;
   each R⁴ is independently halogen, cyano, nitro, C₁-C₃ alkyl, C₁-C₃ haloalkoxy, C₂-C₆ alkenyloxy, C₂-C₆ alkynyloxy, C₂-C₆ cyanoalkoxy, C₂-C₆ alkoxyalkyl or C₂-C₆ alkoxyalkoxy;
   each R⁵ is independently halogen, C₁-C₃ alkyl, C₂-C₆ alkoxyalkyl, C₁-C₃ alkoxy, C₁-C₃ haloalkoxy, C₂-C₆ alkenyloxy, C₂-C₆ alkynyloxy, C₂-C₆ cyanoalkoxy or C₂-C₆ alkoxyalkoxy; provided that at least one R⁵ is selected from halogen;
   m and n are each independently 1, 2 or 3;
   R⁶ is H; or C₁-C₃ alkyl or C₁-C₃ haloalkyl, each optionally substituted with up to 2 substituents independently selected from R^{6a}; or amino, C₂-C₄ alkenyl, C₂-C₄ alkynyl, C₃-C₆ cycloalkyl, CH(═O), S(═O)₂OM, S(═O)ᵤR⁷, (C═W)R⁸ or OR⁹;
   each R^{6a} is independently cyano, C₃-C₆ cycloalkyl, C₁-C₃ alkoxy, C₁-C₃ haloalkoxy, C₁-C₃ alkylthio, C₁-C₃ alkylsulfinyl or C₁-C₃ alkylsulfonyl;
   M is K or Na;
   u is 0, 1 or 2;
   R⁷ is C₁-C₃ alkyl or C₁-C₃ haloalkyl;
   W is O or S;
   R⁸ is C₁-C₃ alkyl, C₂-C₄ alkoxyalkyl, C₂-C₄ alkylaminoalkyl, C₃-C₆ dialkylaminoalkyl, C₁-C₃ alkoxy, C₁-C₃ alkylthio or C₂-C₄ alkylthioalkyl;
   R⁹ is H; or C₁-C₃ alkyl or C₁-C₃ haloalkyl, each optionally substituted with up to 2 substituents independently selected from R^{9a}; or CH(═O), C₃-C₆ cycloalkyl, S(═O)₂OM or (C═W)R¹⁰; and
   each R^{9a} is independently cyano, C₃-C₆ cycloalkyl, C₁-C₃ alkoxy, C₁-C₃ haloalkoxy, C₁-C₃ alkylthio, C₁-C₃ alkylsulfinyl or C₁-C₃ alkylsulfonyl; and
   R¹⁰ is C₁-C₃ alkyl, C₂-C₄ alkoxyalkyl, C2-C4 alkylaminoalkyl, C₃-C₆ dialkylaminoalkyl, C₁-C₃ alkoxy, C₁-C₃ alkylthio or C₂-C₄ alkylthioalkyl; provided that the compound of Formula 1 is not:
N-(2-bromo-4-fluoro-6-nitrophenyl)-4-(2-chloro-4-fluorophenyl)-1,3-dimethyl-1H-pyrazol-5-amine; or
3-chloro-4-(2-chloro-4-fluorophenyl)-N-(2,4-difluoro-6-nitrophenyl)-1-methyl-1H-pyrazol-5-amine;
Embodiment G. The composition of Embodiment F wherein in Formula 1,
   R¹ is methyl;
   R² is cyano, halogen or C₁-C₂ alkyl;
   R³ is halogen;
   each R⁴ is independently halogen, cyano, methyl, C₁-C₂ alkoxy or C₁-C₂ haloalkoxy;
   m is 1 and R⁴ is at the 4-position (or para position); or m is 1 and R⁴ is at the 6-position (or ortho position); or m is 2 and one R⁴ is at the 4-position (or para position), and the other is at the 6-position (or ortho position);
   each R⁵ is independently halogen, methyl, methoxy, halomethyl, C₂-C₄ alkenyloxy or C₂-C₄ cyanoalkoxy;
   n is 1 and R⁵ is at the 4-position (or para position); or n is 1 and R⁵ is at the 6-position (or ortho position); or n is 2 and one R⁵ is at the 4-position (or para position), and the other is at the 6-position (or ortho position); and
   R⁶ is H or methyl.
Embodiment H. The composition of Embodiment G wherein in Formula 1,
   R² is methyl;
   each R⁴ is independently Br, Cl, F, cyano or methoxy;
   each R⁵ is independently Br, Cl, F, methyl or methoxy; and
   R⁶ is H.
Embodiment I. The composition of Embodiment H wherein in Formula 1,
   R⁴ is Br, Cl or F;
   each R⁵ is independently Br, Cl, F or methoxy; and
   m and n are each 1 and R⁴ is at the 4-position and R⁵ is at the 6-position; or m is 1 and R⁴ is at the 4-position, and n is 2 and one R⁵ is at the 4-position and the other is at the 6-position.
Embodiment J. The composition of Embodiment I wherein
   R⁴ is Cl or F; and
   each R⁵ is independently Cl, F or methoxy.
Embodiment K. The composition of anyone of Embodiments A through J wherein component (a) comprises a compound selected from the group consisting of: Compound 1, Compound 18, Compound 19, Compound 23, Compound 57, Compound 60, Compound 68, Compound 72, Compound 73, Compound 93, Compound 111, Compound 112, Compound 121 and Compound 127.
Embodiment L. The composition of Embodiment K wherein component (a) comprises a compound selected from the group consisting of: Compound 68, Compound 72 and Compound 112.
Embodiment M. The composition of Embodiment L wherein component (a) comprises Compound 112.
Embodiment B1. The composition described in the Summary of the Invention (including but not limited to the composition of any one of Embodiments 1 through 108 and A through M) wherein component (b) includes at least one compound selected from (b1) methyl benzimidazole carbamate fungicides such as benomyl, carbendazim, fuberidazole thiabendazole, thiophanate and thiophanate-methyl.
Embodiment B2. The composition described in the Summary of the Invention (including but not limited to the composition of any one of Embodiments 1 through 108 and A through M) wherein component (b) includes at least one compound selected from (b2) dicarboximide fungicides such as chlozolinate, dimethachlone, iprodione, procymidone and vinclozolin.
Embodiment B3. The composition described in the Summary of the Invention (including but not limited to the composition of any one Embodiments 1 through 108 and A through M) wherein component (b) includes at least one compound selected from (b3) demethylation inhibitor fungicides such as triforine, buthiobate, pyrifenox, pyrisoxazole fenarimol, nuarimol, triarimol econazole, imazalil, oxpoconazole, pefurazoate, prochloraz, triflumizoleazaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, mefentrifluconazole, metconazole, myclobutanil, penconazole, propiconazole, ipfentrifluconazole, quinconazole, simeconazole, tebuconazole, tetraconazole triadimefon, triadimenol, triticonazole, uniconazole and uniconazole-P.
Embodiment B4. The composition described in the Summary of the Invention (including but not limited to the composition of any one of Embodiments 1 through 108 and A through M) wherein component (b) includes at least one compound selected from (b4) phenylamide fungicides such as metalaxyl, metalaxyl-M, benalaxyl, benalaxyl-M, furalaxyl, ofurace and oxadixyl.
Embodiment B5. The composition described in the Summary of the Invention (including but not limited to the composition of any one of Embodiments 1 through 108 and A through M) wherein component (b) includes at least one compound selected from (b5) amine/morpholine fungicides such as aldimorph, dodemorph, fenpropimorph, tridemorph, trimorphamide, fenpropidin, piperalin and spiroxamine.
Embodiment B6. The composition described in the Summary of the Invention (including but not limited to the composition of any one of Embodiments 1 through 108 and A through M) wherein component (b) includes at least one compound selected from (b6) phospholipid biosynthesis inhibitor fungicides such as edifenphos, iprobenfos, pyrazophos and isoprothiolane.
Embodiment B7. The composition described in the Summary of the Invention (including but not limited to the composition of any one of Embodiments 1 through 108 and A through M) wherein component (b) includes at least one compound selected from (b7) succinate dehydrogenase inhibitor fungicides such as benodanil, flutolanil, mepronil, isofetamid, fluopyram, fenfuram, carboxin, oxycarboxin thifluzamide, benzovindiflupyr, bixafen, fluindapyr, fluxapyroxad, furametpyr, inpyrfluxam, isopyrazam, penflufen, penthiopyrad, pyrapropoyne, sedaxane, flubeneteram, isoflucypram, pydiflumetofen, boscalid and pyraziflumid.
Embodiment B8. The composition described in the Summary of the Invention (including but not limited to the composition of any one of Embodiments 1 through 108 and A through M) wherein component (b) includes at least one compound selected from (b8) hydroxy(2-amino-)pyrimidine fungicides such as bupirimate, dimethirimol and ethirimol.
Embodiment B9. The composition described in the Summary of the Invention (including but not limited to the composition of any one of Embodiments 1 through 108 and A through M) wherein component (b) includes at least one compound selected from (b9) anilinopyrimidine fungicides such as cyprodinil, mepanipyrim and pyrimethanil.
Embodiment B10. The composition described in the Summary of the Invention (including but not limited to the composition of any one of Embodiments 1 through 108 and A through M) wherein component (b) includes at least one compound selected from (b10) N-phenyl carbamate fungicides such as diethofencarb.
Embodiment B11. The composition described in the Summary of the Invention (including but not limited to the composition of any one of Embodiments 1 through 108 and A through M) wherein component (b) includes at least one compound selected from (b11) fungicides quinone outside inhibitor fungicides such as azoxystrobin, coumoxystrobin, enoxastrobin, flufenoxystrobin, picoxystrobin, pyraoxystrobin, mandestrobin, pyraclostrobin, pyrametostrobin, triclopyricarb, kresoxim-methyl, trifloxystrobin, dimoxystrobin, fenaminstrobin, metominostrobin, orysastrobin, fluoxastrobin, famoxadone, fenamidone and pyribencarb.
Embodiment B12. The composition described in the Summary of the Invention (including but not limited to the composition of any one of Embodiments 1 through 108 and A through M) wherein component (b) includes at least one compound selected from (b12) phenylpyrrole fungicides compound such as fenpiclonil and fludioxonil.
Embodiment B13. The composition described in the Summary of the Invention (including but not limited to the composition of any one of Embodiments 1 through 108 and A through M) wherein component (b) includes at least one compound selected from (b13) azanaphthalene fungicides such as quinoxyfen and proquinazid.
Embodiment B14. The composition described in the Summary of the Invention (including but not limited to the composition of any one of Embodiments 1 through 108 and A through M) wherein component (b) includes at least one compound selected from (b14) cell peroxidation inhibitor fungicides such as biphenyl, chloroneb, dicloran, quintozene, tecnazene, tolclofos-methyl and etridiazole.
Embodiment B15. The composition described in the Summary of the Invention (including but not limited to the composition of any one of Embodiments 1 through 108 and A through M) wherein component (b) includes at least one compound selected from (b15) melanin biosynthesis inhibitors-reductase fungicides such as fthalide, pyroquilon and tricyclazole.
Embodiment B16a. The composition described in the Summary of the Invention (including but not limited to the composition of any one of Embodiments 1 through 108 and A through M) wherein component (b) includes at least one compound selected from (b16a) melanin biosynthesis inhibitors-dehydratase fungicides such as carpropamid, diclocymet, and fenoxanil.
Embodiment B16b. The composition described in the Summary of the Invention (including but not limited to the composition of any one of Embodiments 1 through 108 and A through M) wherein component (b) includes at least one compound selected from (b16b) melanin biosynthesis inhibitor-polyketide synthase fungicides such as tolprocarb.
Embodiment B17. The composition described in the Summary of the Invention (including but not limited to the composition of any one of Embodiments 1 through 108 and A through M) wherein component (b) includes at least one compound selected from (b17) keto reductase inhibitor fungicides such as fenhexamid, fenpyrazamine, quinofumelin and ipflufenoquin.
Embodiment B18. The composition described in the Summary of the Invention (including but not limited to the composition of any one of Embodiments 1 through 108 and A through M) wherein component (b) includes at least one compound selected from (b18) squalene-epoxidase inhibitor fungicides such as pyributicarb, naftifine and terbinafine.
Embodiment B19. The composition described in the Summary of the Invention (including but not limited to the composition of any one of Embodiments 1 through 108 and A through M) wherein component (b) includes at least one compound selected from (b19) polyoxin fungicides such as polyoxin.
Embodiment B20. The composition described in the Summary of the Invention (including but not limited to the composition of any one of Embodiments 1 through 108 and A through M) wherein component (b) includes at least one compound selected from (b20) phenylurea fungicides such as pencycuron.
Embodiment B21. The composition described in the Summary of the Invention (including but not limited to the composition of any one of Embodiments 1 through 108 and A through M) wherein component (b) includes at least one compound selected from (b21) quinone inside inhibitor fungicides such as cyazofamid, amisulbrom and fenpicoxamid (Registry Number 517875-34-2).
Embodiment B22. The composition described in the Summary of the Invention (including but not limited to the composition of any one of Embodiments 1 through 108 and A through M) wherein component (b) includes at least one compound selected from (b22) benzamide and thiazole carboxamide fungicides such as zoxamide and ethaboxam.
Embodiment B23. The composition described in the Summary of the Invention (including but not limited to the composition of any one of Embodiments 1 through 108 and A through M) wherein component (b) includes at least one compound selected from (b23) enopyranuronic acid antibiotic fungicides such as blasticidin-S.
Embodiment B24. The composition described in the Summary of the Invention (including but not limited to the composition of any one of Embodiments 1 through 108 and A through M) wherein component (b) includes at least one compound selected from (b24) hexopyranosyl antibiotic fungicides such as kasugamycin.
Embodiment B25. The composition described in the Summary of the Invention (including but not limited to the composition of any one of Embodiments 1 through 108 and A through M) wherein component (b) includes at least one compound selected from (b25) glucopyranosyl antibiotic: protein synthesis fungicides such as streptomycin.
Embodiment B26. The composition described in the Summary of the Invention (including but not limited to the composition of any one Embodiments 1 through 108 and A through M) wherein component (b) includes at least one compound selected from (b26) glucopyranosyl antibiotic: trehalase and inositol biosynthesis fungicides such as validamycin.
Embodiment B27. The composition described in the Summary of the Invention (including but not limited to the composition of any one of Embodiments 1 through 108 and A through M) wherein component (b) includes at least one compound selected from (b27) cyanoacetylamideoxime fungicides such as cymoxanil.
Embodiment B28. The composition described in the Summary of the Invention (including but not limited to the composition of any one of Embodiments 1 through 108 and A through M) wherein component (b) includes at least one compound selected from (b28) carbamate fungicides such as propamacarb, prothiocarb and iodocarb.
Embodiment B29. The composition described in the Summary of the Invention (including but not limited to the composition of any one of Embodiments 1 through 108 and A through M) wherein component (b) includes at least one compound selected from (b29) oxidative phosphorylation uncoupling fungicides such as fluazinam, binapacryl, meptyldinocap and dinocap.
Embodiment B30. The composition described in the Summary of the Invention (including but not limited to the composition of any one of Embodiments 1 through 108 and A through M) wherein component (b) includes at least one compound selected from (b30) organo tin fungicides such as fentin acetate, fentin chloride and fentin hydroxide.
Embodiment B31. The composition described in the Summary of the Invention (including but not limited to the composition of any one of Embodiments 1 through 108 and A through M) wherein component (b) includes at least one compound selected from (b31) carboxylic acid fungicides such as oxolinic acid.
Embodiment B32. The composition described in the Summary of the Invention (including but not limited to the composition of any one of Embodiments 1 through 108 and A through M) wherein component (b) includes at least one compound selected from (b32) heteroaromatic fungicides such as hymexazole and octhilinone.
Embodiment B33. The composition described in the Summary of the Invention (including but not limited to the composition of any one of Embodiments 1 through 108 and A through M) wherein component (b) includes at least one compound selected from (b33) phosphonate fungicides such as phosphorous acid and its various salts, including fosetyl-aluminum.
Embodiment B34. The composition described in the Summary of the Invention (including but not limited to the composition of any one of Embodiments 1 through 108 and A through M) wherein component (b) includes at least one compound selected from (b34) phthalamic acid fungicides such as teclofthalam.
Embodiment B35. The composition described in the Summary of the Invention (including but not limited to the composition of any one of Embodiments 1 through 108 and A through M) wherein component (b) includes at least one compound selected from (b35) benzotriazine fungicides such as triazoxide.
Embodiment B36. The composition described in the Summary of the Invention (including but not limited to the composition of any one of Embodiments 1 through 108 and A through M) wherein component (b) includes at least one compound selected from (b36) benzene-sulfonamide fungicides such as flusulfamide.
Embodiment B37. The composition described in the Summary of the Invention (including but not limited to the composition of any one of Embodiments 1 through 108 and A through M) wherein component (b) includes at least one compound selected from (b37) pyridazinone fungicides such as diclomezine.
Embodiment B38. The composition described in the Summary of the Invention (including but not limited to the composition of any one of Embodiments 1 through 108 and A through M) wherein component (b) includes at least one compound selected from (b38) thiophene-carboxamide fungicides such as silthiofam.
Embodiment B39. The composition described in the Summary of the Invention (including but not limited to the composition of any one of Embodiments 1 through 108 and A through M) wherein component (b) includes at least one compound selected from (b39) complex I NADH oxidoreductase inhibitor fungicides such as diflumetorim, tolfenpyrad and fenazaquin.
Embodiment B40. The composition described in the Summary of the Invention (including but not limited to the composition of any one Embodiments 1 through 108 and A through M) wherein component (b) includes at least one compound selected from (b40) carboxylic acid amide fungicides such as dimethomorph, benthiavalicarb, benthiavalicarb-isopropyl, iprovalicarb, valifenalate, mandipropamid, flumorph, dimethomorph, flumorph, pyrimorph, benthiavalicarb, benthiavalicarb-isopropyl, iprovalicarb, tolprocarb, valifenalate and mandipropamid.
Embodiment B41. The composition described in the Summary of the Invention (including but not limited to the composition of any one of Embodiments 1 through 108 and A through M) wherein component (b) includes at least one compound selected from (b41) tetracycline antibiotic fungicides such as oxytetracycline.
Embodiment B42. The composition described in the Summary of the Invention (including but not limited to the composition of any one of Embodiments 1 through 108 and A through M) wherein component (b) includes at least one compound selected from (b42) thiocarbamate fungicides such as methasulfocarb.
Embodiment B43. The composition described in the Summary of the Invention (including but not limited to the composition of any one of Embodiments 1 through 108 and A through M) wherein component (b) includes at least one compound selected from (b43) benzamide fungicides such as fluopicolide and fluopimomide.
Embodiment B44. The composition described in the Summary of the Invention (including but not limited to the composition of any one of Embodiments 1 through 108 and A through M) wherein component (b) includes at least one compound selected from (b44) microbial fungicides such as Bacillus amyloliquefaciens strains QST713, FZB24, MB1600, D747, F727, TJ100 (also called strain 1 BE; known from EP2962568) and the fungicidal lipopeptides which they produce.
Embodiment B45. The composition described in the Summary of the Invention (including but not limited to the composition of any one of Embodiments 1 through 108 and A through M) wherein component (b) includes at least one compound selected from (b45) quinone outside inhibitor, stigmatellin binding fungicides such as ametoctradin.
Embodiment B46. The composition described in the Summary of the Invention (including but not limited to the composition of any one of Embodiments 1 through 108 and A through M) wherein component (b) includes at least one compound selected from (b46) plant extract fungicides such as Melaleuca alternifolia, eugenol, geraniol and thymol.
Embodiment B47. The composition described in the Summary of the Invention (including but not limited to the composition of any one of Embodiments 1 through 108 and A through M) wherein component (b) includes at least one compound selected from (b47) cyanoacrylate fungicides such as phenamacril.
Embodiment B48. The composition described in the Summary of the Invention (including but not limited to the composition of any one of Embodiments 1 through 108 and A through M) wherein component (b) includes at least one compound selected from (b48) polyene fungicides such as natamycin.
Embodiment B49. The composition described in the Summary of the Invention (including but not limited to the composition of any one of Embodiments 1 through 108 and A through M) wherein component (b) includes at least one compound selected from (b49) oxysterol binding protein inhibitor fungicides such as oxathiapiprolin and fluoxapiprolin.
Embodiment B50. The composition described in the Summary of the Invention (including but not limited to the composition of any one of Embodiments 1 through 108 and A through M) wherein component (b) includes at least one compound selected from (b50) aryl-phenyl-ketone fungicides such as metrafenone and pyriofenone.
Embodiment B51. The composition described in the Summary of the Invention (including but not limited to the composition of any one of Embodiments 1 through 108 and A through M) wherein component (b) includes at least one compound selected from (b51) host plant defense induction fungicides such as acibenzolar-S-methyl, probenazole, tiadinil, isotianil, laminarin, extract from Reynoutria sachalinensis and Bacillus mycoides isolate J and cell walls of Saccharomyces cerevisiae strain LAS117.
Embodiment B52. The composition described in the Summary of the Invention (including but not limited to the composition of any one of Embodiments 1 through 108 and A through M) wherein component (b) includes at least one compound selected from (b52) multi-site activity fungicides such as copper oxychloride, copper sulfate, copper hydroxide, Bordeaux composition (tribasic copper sulfide), elemental sulfur, ferbam, mancozeb, maneb, metiram, propineb, thiram, zinc thiazole, zineb, ziram, folpet, captan, captafol, chlorothalonil, dichlofluanid, tolyfluanid, guazatine, iminoctadine albesilate, iminoctadine triacetate, anilazine, dithianon, quinomethionate and fluoroimide.
Embodiment B53. The composition described in the Summary of the Invention (including but not limited to the composition of any one of Embodiments 1 through 108 and A through M) wherein component (b) includes at least one compound selected from (b53) biological fungicides with multiple modes of action such as extract from the cotyledons of lupine plantlets.
Embodiment B54. The composition described in the Summary of the Invention (including but not limited to the composition of any one Embodiments 1 through 108 and A through M) wherein component (b) includes at least one compound selected from (b54) fungicides other than fungicides of component (a) and components (b1) through (b53), such as cyflufenamid, bethoxazin, neo-asozin, pyrrolnitrin, tebufloquin, dodine, flutianil, ferimzone, picarbutrazox, dichlobentiazox (Registry Number 957144-77-3), dipymetitrone (Registry Number 16114-35-5), flometoquin, tolnifanide (Registry Number 304911-98-6), N-[4-[4-chloro-3-(trifluoromethyl)phenoxy]-2,5-dimethylphenyl]-N-ethyl-N-methylmethanimidamide, 5-fluoro-2-[(4-fluoro-phenyl)methoxy]-4-pyrimidinamine and 4-fluorophenyl N-[1-[[[1-(4-cyanophenyl)-ethyl]sulfonyl]methyl]propyl]carbamate (XR-539).
Embodiment B55. The composition described in the Summary of the Invention (including but not limited to the composition of any one of Embodiments 1 through 108 and A through M) wherein component (b) includes (1S)-2,2-bis(4-fluorophenyl)-1-methylethyl N-[[3-(acetyloxy)-4-methoxy-2-pyridinyl]carbonyl]-L-alaninate (provisional common name florylpicoxamid).
Embodiment B56. The composition described in the Summary of the Invention (including but not limited to the composition of any one of Embodiments 1 through 108 and A through M) wherein component (b) includes 1-[2-[[[1-(4-chlorophenyl)-1H-pyrazol-3-yl]oxy]methyl]-3-methylphenyl]-1,4-dihydro-4-methyl-5H-tetrazol-5-one (provisional common name metyltetraprole).
Embodiment B57. The composition described in the Summary of the Invention (including but not limited to the composition of any one of Embodiments 1 through 108 and A through M) wherein component (b) includes 3-chloro-4-(2,6-difluorophenyl)-6-methyl-5-phenylpyridazine (provisional common name pyridachlometyl).
Embodiment B58. The composition described in the Summary of the Invention (including but not limited to the composition of any one of Embodiments 1 through 108 and A through M) wherein component (b) includes (4-phenoxyphenyl)methyl 2-amino-6-methyl-pyridine-3-carboxylate (provisional common name aminopyrifen).
Embodiment B59. The composition described in the Summary of the Invention (including but not limited to the composition of any one of Embodiments 1 through 108 and A through M) wherein component (b) includes at least one compound selected from (b54.11) (i.e Formula b54.11)

Component (b54.11) Relates to a Compound of Formula b54.11

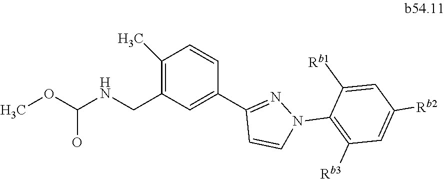

wherein
R^{b1} and R^{b3} are each independently halogen; and
R^{b2} is H, halogen, C₁-C₃ alkyl, C₁-C₃ haloalkyl or C₃-C₆ cycloalkyl.
Embodiment B60. The composition of Embodiment B59 wherein component (b) includes at least one fungicidal compound selected from the group consisting of methyl N-[[5-[1-(2,6-difluoro-4-formylphenyl)-1H-pyrazol-3-yl]-2-methylphenyl] methyl]carbamate, methyl N-[[5-[1-(4-cyclopropyl-2,6-dichlorophenyl)-1H-pyrazol-3-yl]-2-methylphenyl]-methyl]carbamate, methyl N-[[5-[1-(4-chloro-2,6-difluorophenyl)-1H-pyrazol-3-yl]-2-methylphenyl]methyl]carbamate, methyl N-[[5-[1-(4-cyclopropyl-2,6-difluorophenyl)-1H-pyrazol-3-yl]-2-methylphenyl]methyl]carbamate, methyl N-[[5-[1-[2,6-difluoro-4-(1-methylethyl)phenyl]-1H-pyrazol-3-yl]-2-methylphenyl]methyl]carbamate and methyl N-[[5-[1-[2,6-difluoro-4-(trifluoromethyl)phenyl]-1H-pyrazol-3-yl]-2-methylphenyl]methyl]carbamate.
Embodiment B60b. The composition of Embodiment B60 wherein component (b) includes at least one fungicidal compound selected from the group consisting of methyl N-[[5-[1-(4-cyclopropyl-2,6-dichlorophenyl)-1H-pyrazol-3-yl]-2-methylphenyl]methyl]-carbamate, methyl N-[[5-[1-(4-chloro-2,6-difluorophenyl)-1H-pyrazol-3-yl]-2-methyl-phenyl]methyl]carbamate, methyl N-[[5-[1-[2,6-difluoro-4-(1-methylethyl)phenyl]-1H-pyrazol-3-yl]-2-methylphenyl]methyl]carbamate and methyl N-[[5-[1-[2,6-difluoro-4-(trifluoromethyl)phenyl]-1H-pyrazol-3-yl]-2-methylphenyl]methyl]carbamate.
Embodiment B61. The composition described in the Summary of the Invention (including but not limited to the composition of any one of Embodiments 1 through 108 and A through M) wherein component (b) includes at least one compound selected from (b54.12) (i.e Formula b54.12) Component (b54.12) relates to a compound of Formula b54.12

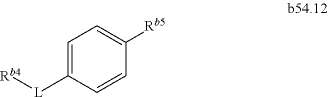

wherein
R^{b4} is

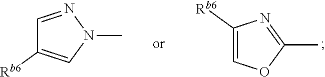

R^{b6} is C₂-C₄ alkoxycarbonyl or C₂-C₄ haloalkylaminocarbonyl;
L is CH₂ or CH₂O, wherein the atom to the right is connected to the phenyl ring in Formula b54.12;
R^{b5} is

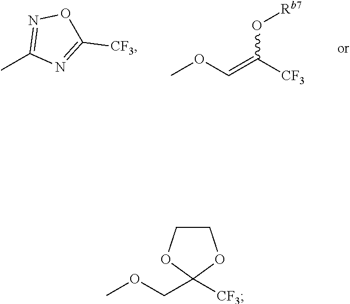

and
R^{b7} is C₁-C₃ alkyl, wherein the wavy bond indicates the adjacent double bond is either (Z)- or (E)-configuration, or a mixture thereof.
Embodiment B62. The composition of Embodiment B61 wherein component (b) includes at least one fungicidal compound selected from the group consisting of N-(2,2,2-trifluoroethyl)-2-[[4-[5-(trifluoromethyl)-1,2,4-oxadiazol-3-yl]phenyl]methyl]-4-oxazolecarboxamide, ethyl 1-[[4-[5-(trifluoromethyl)-1,2,4-oxadiazol-3-yl]phenoxy]methyl]-1H-pyrazole-4-carboxylate, ethyl 1-[[4-[[(1Z)-2-ethoxy-3,3,3-trifluoro-1-propen-1-yl]oxy]phenyl]methyl]-1H-pyrazole-4-carboxylate and ethyl 1-[[4-[[2-(trifluoromethyl)-1,3-dioxolan-2-yl]methoxy]phenyl]methyl]-1H-pyrazole-4-carboxylate.
Embodiment B62b. The composition of Embodiment B62 wherein component (b) includes at least one fungicidal compound selected from the group consisting of N-(2,2,2-trifluoroethyl)-2-[[4-1-[5-(trifluoromethyl)-1,2,4-oxadiazol-3-yl]phenyl]methyl]-4-oxazolecarboxamide and ethyl 1-[[4-[[(1Z)-2-ethoxy-3,3,3-trifluoro-1-propen-1-yl]oxy]phenyl]methyl]-1H-pyrazole-4-carboxylate.
Embodiment B63. The composition described in the Summary of the Invention (including but not limited to the composition of any one of Embodiments 1 through 108 and A through M) wherein component (b) includes at least one fungicidal compound (fungicide) selected from the group consisting of azoxystrobin, benzovindiflupyr, boscalid (nicobifen), bixafen, bromuconazole, carbendazim, chlorothalonil, copper hydroxide, cyflufenamid, cyproconazole, difenoconazole, dimoxystrobin, epoxiconazole, famoxadone, fenbuconazole, fenpropidin, fenpropimorph, fluindapyr, flusilazole, flutriafol, fluxapyroxad, hexaconazole, ipconazole kresoxim-methyl, manzate, metconazole, metominostrobin, metrafenone, myclobutanil, penconazole, penthiopyrad, picoxystrobin, prochloraz, propiconazole, proquinazid, prothioconazole, pydiflumetofen, pyraclostrobin, pyrametostrobin, pyraoxystrobin, pyriofenone quinoxyfen, tebuconazole, trifloxystrobin, triticonazole, methyl N-[[5-[1-(4-cyclopropyl-2,6-dichlorophenyl)-1H-pyrazol-3-yl]-2-methylphenyl]methyl]carbamate, methyl N-[[5-[1-(4-chloro-2,6-difluorophenyl)-1H-pyrazol-3-yl]-2-methylphenyl]-methyl]carbamate, methyl N-[[5-[1-[2,6-difluoro-4-(1-methylethyl)phenyl]-1H-pyrazol-3-yl]-2-methylphenyl]methyl]carbamate, methyl N-[[5-[1-[2,6-difluoro-4-(trifluoromethyl)phenyl]-1H-pyrazol-3-yl]-2-methylphenyl]methyl]carbamate, N-(2,2,2-trifluoroethyl)-2-[[4-1-[5-(trifluoromethyl)-1,2,4-oxadiazol-3-yl]phenyl]methyl]-4-oxazolecarboxamide and ethyl 1-[[4-[[(1Z)-2-ethoxy-3,3,3-trifluoro-1-propen-1-yl]oxy]phenyl]methyl]-1H-pyrazole-4-carboxylate.
Embodiment B64. The composition of Embodiment B63 wherein component (b) includes at least one compound selected from the group consisting of azoxystrobin, benzovindiflupyr, bixafen, chlorothalonil, copper hydroxide, cyflufenamid, cyproconazole, difenoconazole, dimoxystrobin, epoxiconazole, famoxadone, fenpropidin, fenpropimorph, fluindapyr, flusilazole, flutriafol, fluxapyroxad, kresoxim-methyl, manzate, metconazole, metominostrobin, metrafenone, myclobutanil, penthiopyrad, picoxystrobin, propiconazole, proquinazid, prothioconazole, pydiflumetofen, pyraclostrobin, pyrametostrobin, pyraoxystrobin, pyriofenone, quinoxyfen, tebuconazole, trifloxystrobin, triticonazole, methyl N-[[5-[1-(4-cyclopropyl-2,6-dichlorophenyl)-1H-pyrazol-3-yl]-2-methylphenyl]methyl]carbamate, methyl N-[[5-[1-(4-chloro-2,6-difluorophenyl)-1H-pyrazol-3-yl]-2-methylphenyl]-methyl]carbamate, methyl N-[[5-[1-[2,6-difluoro-4-(1-methylethyl)phenyl]-1H-pyrazol-3-yl]-2-methylphenyl]methyl]carbamate, methyl N-[[5-[1-[2,6-difluoro-4-(trifluoromethyl)phenyl]-1H-pyrazol-3-yl]-2-methylphenyl]methyl]carbamate, N-(2,2,2-trifluoroethyl)-2-[[4-[5-(trifluoromethyl)-1,2,4-oxadiazol-3-yl]phenyl]methyl]-4-oxazolecarboxamide and ethyl 1-[[4-[[(1Z)-2-ethoxy-3,3,3-trifluoro-1-propen-1-yl]oxy]phenyl]methyl]-1H-pyrazole-4-carboxylate.
Embodiment B65. The composition of Embodiment B64 wherein component (b) includes at least one compound selected from the group consisting of azoxystrobin, benzovindiflupyr, bixafen, chlorothalonil, copper hydroxide, cyproconazole, difenoconazole, epoxiconazole, fenpropidin, fenpropimorph, fluindapyr, flutriafol, fluxapyroxad, manzate, metominostrobin, picoxystrobin, prothioconazole, pydiflumetofen, pyraclostrobin, tebuconazole, trifloxystrobin, methyl N-[[5-[1-(4-cyclopropyl-2,6-dichlorophenyl)-1H-pyrazol-3-yl]-2-methylphenyl]methyl]carbamate, methyl N-[[5-[1-(4-chloro-2,6-difluorophenyl)-1H-pyrazol-3-yl]-2-methylphenyl]-methyl]carbamate, methyl N-[[5-[1-[2,6-difluoro-4-(1-methylethyl)phenyl]-1H-pyrazol-3-yl]-2-methylphenyl]methyl]carbamate, methyl N-[[5-[1-[2,6-difluoro-4-(trifluoromethyl)phenyl]-1H-pyrazol-3-yl]-2-methylphenyl]methyl]carbamate, N-(2,2,2-trifluoroethyl)-2-[[4-[5-(trifluoromethyl)-1,2,4-oxadiazol-3-yl]phenyl]methyl]-4-oxazolecarboxamide and ethyl 1-[[4-[[(1Z)-2-ethoxy-3,3,3-trifluoro-1-propen-1-yl]oxy]phenyl]methyl]-1H-pyrazole-4-carboxylate.
Embodiment B66. The composition of Embodiment B65 wherein component (b) includes at least one compound selected from the group consisting of azoxystrobin, benzovindiflupyr, bixafen, chlorothalonil, copper hydroxide, cyproconazole, epoxiconazole, fenpropidin, fenpropimorph, fluindapyr, flutriafol, fluxapyroxad, manzate, metominostrobin, picoxystrobin, prothioconazole, pydiflumetofen, pyraclostrobin, tebuconazole, trifloxystrobin.

Of note is the composition of any one of the embodiments described herein, including any Embodiments 1 through 108, A through M, and B1 through B66, wherein reference to Formula 1 includes salts thereof but not N-oxides thereof; therefore the phrase “a compound of Formula 1” can be replaced by the phrase “a compound of Formula 1 or a salt thereof”. In this composition of note, component (a) comprises a compound of Formula 1 or a salt thereof.

Also noteworthy as embodiments are fungicidal compositions of the present invention comprising a fungicidally effective amount of a composition of Embodiments 1 through 108, A through M, and B1 through B66, and at least one additional component selected from the group consisting of surfactants, solid diluents and liquid diluents.
Embodiments of the invention further include methods for controlling plant diseases caused by fungal plant pathogens comprising applying to the plant or portion thereof, or to the plant seed or seedling, a fungicidally effective amount of a composition any one of Embodiments 1 through 108, A through M, and B1 through B66 (e.g., as a composition including formulation ingredients as described herein). Embodiments of the invention also include methods for protecting a plant or plant seed from diseases caused by fungal pathogens comprising applying a fungicidally effective amount of a composition of any one of Embodiments 1 through 108, A through M, and B1 through B66 to the plant or plant seed.

Some embodiments of the invention involve control of a plant disease or protection from a plant disease that primarily afflicts plant foliage and/or applying the composition of the invention to plant foliage (i.e. plants instead of seeds). The preferred methods of use include those involving the above preferred compositions; and the diseases controlled with particular effectiveness include plant diseases caused by fungal plant pathogens.

Combinations of fungicides used in accordance with this invention can facilitate disease control and retard resistance development.

Method embodiments further include:
Embodiment C1. A method for protecting a plant from a disease selected from rust, powdery mildew and Septoria diseases comprising applying to the plant a fungicidally effective amount of the composition comprising components (a) and (b) described in the Summary of the Invention or any one of Embodiments 1 through 108.
Embodiment C2. The method of Embodiment Cl wherein the disease is a rust disease and component (b) of the composition includes at least one fungicidal compound selected from (b3) demethylation inhibitor (DMI) fungicides, (b5) amine/morpholine fungicides, (b7) succinate dehydrogenase inhibitor fungicides, (b11) quinone outside inhibitor (QoI) fungicides, (b13) methyl benzimidazole carbamate fungicides and (b52) multi-site activity fungicides.
Embodiment C3. The method of Embodiment C2 wherein component (b) of the composition includes at least one fungicidal compound selected from (b3) demethylation inhibitor (DMI) fungicides, (b7) succinate dehydrogenase inhibitor fungicides and (b11) quinone outside inhibitor (QoI) fungicides.
Embodiment C4. The method of Embodiment C3 wherein component (b) of the composition includes at least one fungicidal compound selected from (b3) demethylation inhibitor (DMI) fungicides, (b7) succinate dehydrogenase inhibitor fungicides and (b11) quinone outside inhibitor (QoI).
Embodiment C5. The method of any one of Embodiments Cl through C4 wherein component (b) of the composition includes at least one fungicidal compound selected from the group consisting of azoxystrobin, benzovindiflupyr, bixafen, cyproconazole, difenoconazole, epoxiconazole, fenpropimorph, florylpicoxamid, fluindapyr, flutriafol, fluxapyroxad, inpyrfluxam, isoflucypram, mefentrifluconazole, metominostrobin, picoxystrobin, prothioconazole, pydiflumetofen, pyraclostrobin, tebuconazole and trifloxystrobin.
Embodiment C6. The method of Embodiment C5 wherein component (b) of the composition includes at least one fungicidal compound selected from the group consisting of azoxystrobin, benzovindiflupyr, cyproconazole, epoxiconazole, fluindapyr, fluxapyroxad, metominostrobin, picoxystrobin, prothioconazole, pyraclostrobin, tebuconazole and trifloxystrobin.
Embodiment C7. The method of any one of Embodiments C2 through C6 wherein the disease is Asian soybean rust caused by Phakopsora pachyrhizi.
Embodiment C8. The method of any one of Embodiments C2 through C6 wherein the disease is wheat leaf rust caused by Puccinia recondita.
Embodiment C9. The method of Embodiment Cl wherein the disease is a powdery mildew disease and component (b) of the composition includes at least one fungicidal compound selected from (b3) demethylation inhibitor (DMI) fungicides, (b11) quinine outside inhibitor (QoI) fungicides, (b13) azanaphthalene fungicides and (b52) multi-site activity fungicides.
Embodiment C10. The method of Embodiment C9 wherein component (b) of the composition includes at least one fungicidal compound selected from (b3) demethylation inhibitor (DMI) fungicides, (b11) quinone outside inhibitor (QoI) fungicides and (b52) multi-site activity fungicides.
Embodiment C11. The method of Embodiments C9 and C10 wherein component (b) of the composition includes at least one fungicidal compound selected from the group consisting of azoxystrobin, chlorothalonil, copper sulfate, cyproconazole, difenoconazole, epoxiconazole, fenpropimorph, florylpicoxamid, flutriafol, mancozeb, mefentrifluconazole, metominostrobin, picoxystrobin, prothioconazole, pyraclostrobin, tebuconazole and trifloxystrobin.
Embodiment C12. The method of Embodiment C11 wherein component (b) of the composition includes at least one fungicidal compound selected from the group consisting of cyproconazole, difenoconazole, epoxiconazole, prothioconazole and tebuconazole.
Embodiment C13. The method of Embodiment C10 wherein component (b) of the composition includes at least one fungicidal compound selected from (b3) DMI fungicides.
Embodiment C14. The method of Embodiment C13 wherein component (b) of the composition includes at least one fungicidal compound selected from the group consisting of cyproconazole, difenoconazole and prothioconazole.
Embodiment C15. The method Embodiment C10 wherein component (b) of the composition includes at least one fungicidal compound selected from (b11) QoI fungicides.
Embodiment C16. The method of Embodiment C15 wherein component (b) of the composition includes at least one fungicidal compound selected from the group consisting of azoxystrobin, picoxystrobin and pyraclostrobin.
Embodiment C17. The method of any one of Embodiments C9 through C16 wherein the disease is wheat powdery mildew caused by Erysiphe graminis.
Embodiment C18. The method of Embodiment Cl wherein the disease is a Septoria disease and component (b) of the composition includes at least one fungicidal compound selected from (b3) demethylation inhibitor (DMI) fungicides and (b11) quinine outside inhibitor (QoI) fungicides.
Embodiment C19. The method of Embodiment C18 wherein component (b) of the composition includes at least one fungicidal compound selected from the group consisting of azoxystrobin, cyproconazole, difenoconazole, epoxiconazole, fenpropimorph, florylpicoxamid, flutriafol, mefentrifluconazole, metominostrobin, picoxystrobin, prothioconazole, pyraclostrobin, tebuconazole and trifloxystrobin.
Embodiment C20. The method of Embodiment C19 wherein component (b) of the composition includes at least one fungicidal compound selected from the group consisting of epoxiconazole and fenpropimorph.
Embodiment C21. The method of any one of Embodiments C18 through C20 wherein the disease is wheat leaf blotch caused by Zymoseptoria tritici.
Embodiment C22. The method of Embodiment Cl wherein the disease is a Botrytis disease and component (b) of the composition includes at least one fungicidal compound selected from (b11) quinone outside inhibitor (QoI) fungicides and (b52) multi-site activity fungicides.
Embodiment C23. The method of Embodiment C22 wherein component (b) of the composition includes at least one fungicidal compound selected from the group consisting of azoxystrobin, chlorothalonil, florylpicoxamid, mancozeb, metominostrobin, picoxystrobin, pyraclostrobin and trifloxystrobin.
Embodiment C24. The method of Embodiment C23 wherein component (b) of the composition includes at least one fungicidal compound selected from the group consisting of azoxystrobin and chlorothalonil.
Embodiment C25. The method of any one of Embodiments Cl through C24 wherein components (a) and (b) are applied in synergistically effective amounts (and in a synergistic ratio relative to each other).

Of note are embodiments that are counterparts of Embodiments Cl through C25 relating to a method for controlling plant diseases caused by fungal plant pathogens comprising applying to the plant or portion thereof, a fungicidally effective amount of a fungicidal composition of the invention.

As noted in the Summary of the Invention, this invention also relates to a compound of Formula 1, or an N-oxide or salt thereof. Also noted is that the embodiments of this invention, including Embodiments 1-108, relate also to compounds of Formula 1.

This invention provides a fungicidal composition comprising a compound of Formula 1 (including all stereoisomers, N-oxides, and salts thereof), and at least one other fungicide.

Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as embodiments of such methods are methods comprising applying a fungicidally effective amount of a compound corresponding to any of the compound embodiments describe above. Of particular note are embodiments where the compounds are applied as compositions of this invention.

Of note are compounds of Formula 1 that are compounds of Formula 1A (including all geometric and stereoisomers), N-oxides, hydrates and salts thereof, and agricultural compositions containing them and their use as fungicides:

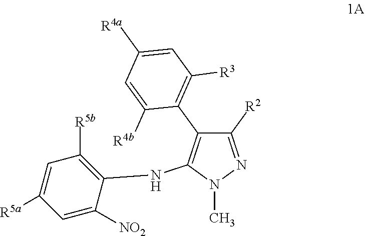

wherein
R² is cyano, halogen or C₁-C₂ alkyl;
R³ is halogen;
R^{4a} and R^{4b} are each independently H or halogen, provided that at least one is halogen; and
R^{5a} and R^{5b} are each independently H, halogen, methyl or methoxy, provided that at least one is halogen;
provided that when R³ is Cl, R^{4a} is F and R^{4b} is H, then R^{5a} is H, Br, Cl, I, methyl or methoxy.

Embodiment A1. A compound of Formula 1A wherein
R² is methyl or ethyl;
R³ is Br, Cl or F;
R^{4a} and R^{4b} are each independently H, Br, Cl or F; and
R^{5a} and R^{5b} are each independently H, Br, Cl, F or methyl.

Embodiment B1. A compound of Embodiment A1 wherein
R² is methyl;
R^{4a} is Cl or F;
R^{4b} is H, Cl or F; and
R^{5a} is H, Cl, F or methyl; and
R^{5b} is H or F.

Also of note is a fungicidal composition comprising a fungicidally effective amount of a compound of Formula 1A (including all geometric and stereoisomers, N-oxides, and salts thereof) or any one of counterpart embodiments that are embodiment counterparts to Embodiments 1 through 107 and Embodiments A through M and at least one additional component selected from the group consisting of surfactants, solid diluents and liquid diluents. Additionally, of note is a method for controlling plant diseases caused by fungal plant pathogens comprising applying to the plant or portion thereof, or to the plant seed, a fungicidally effective amount of a compound of Formula 1A (including all geometric and stereoisomers, N-oxides, and salts thereof) or any one of said counterpart embodiments. Of particular note are embodiments where the compounds of Formula 1A are applied as compositions of this invention.

One or more of the following methods and variations as described in Schemes 1-12 can be used to prepare the compounds of Formula 1. The definitions of R¹, R², R³, R⁴, R⁵, m, n and R⁶ in the compounds of Formulae 1-21 below are as defined above in the Summary of the Invention unless otherwise noted. Formulae 1a and 1b are subsets of Formula 1. Substituents for subset formulae are as defined for its parent formula unless otherwise noted.

As shown in Scheme 1, compounds of Formula 1 can be prepared by reaction of 5-aminopyrazoles of Formula 2 with nitrophenyl compounds of Formula 3 wherein L¹ is a leaving group such as halogen (e.g., F, Cl, Br, I) or sulfonate (e.g., mesylate, triflate or p-toluenesulfonate), optionally in the presence of a metal catalyst, and generally in the presence of a base such as potassium tert-butoxide, triethylamine or potassium carbonate and a solvent such as tetrahydrofuran, N,N-dimethylformamide, 1,4-dioxane, toluene, ethanol, methanol or dimethyl sulfoxide. In certain instances, the use of a metal catalyst in amounts ranging from catalytic up to superstoichiometric can facilitate the desired reaction. Typical reaction conditions include, for example, running the reaction in the presence of a metal catalyst such as copper salt complexes (e.g., CuI with N,N′-dimethylethylenediamine, proline or bipyridyl), palladium complexes (e.g., tris(dibenzylideneacetone)dipalladium(0)) or palladium salts (e.g., palladium acetate) with ligands such as 4,5-bis(diphenylphosphino)-9,9-dimethylxanthene, 2-dicyclohexylphosphino-2′,4′,6′-triisopropylbiphenyl or 2,2′-bis-(diphenylphosphino)1,1′-binaphthalene, with a base such as potassium carbonate, cesium carbonate, potassium phosphate, sodium phenoxide or sodium tert-butoxide and a solvent such as N,N-dimethylformamide, 1,2-dimethoxyethane, dimethyl sulfoxide, 1,4-dioxane or toluene, optionally containing an alcohol such as ethanol. For relevant references, see PCT Patent Publications WO 2013/126283, Synthesis Example 1, Step C; and WO 2010/020363, Example 2A. Also, the method of Scheme 1 is illustrated in present Example 1, Step C; Example 5, Step C; and Example 3. Compounds of Formula 3 are commercially available, or their preparation is known in the art.

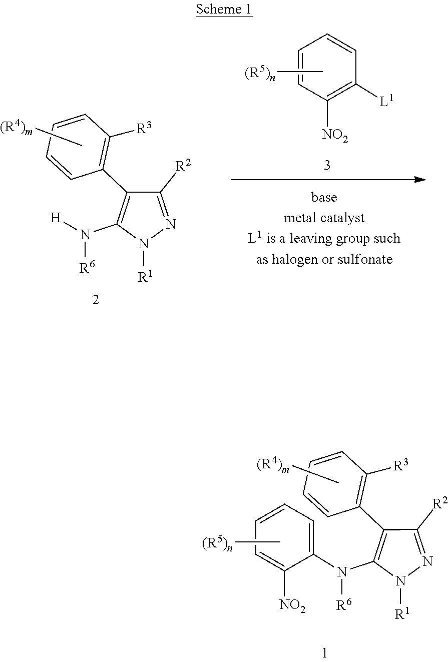

General methods useful for preparing 5-aminopyrazoles of Formula 2 are well-known in the art; see, for example, Journal für Praktische Chemie (Leipzig) 1911, 83, 171 and J. Am. Chem. Soc. 1954, 76, 501. One such method is illustrated in Scheme 2 below, wherein 5-aminopyrazoles of Formula 2 are prepared by condensing compounds of Formula 4 with a hydrazine of Formula 5 (e.g., methylhydrazine or ethylhydrazine) in a solvent such as ethanol or methanol and optionally in the presence of an acid such as acetic acid, according to general procedures known in the art; see, for example, PCT Patent Publication WO 2012/031061 Synthesis Example 1, Step A; and Synthesis Example 2, Step C. Also, the method of Scheme 2 is illustrated in present Example 1, Step B.

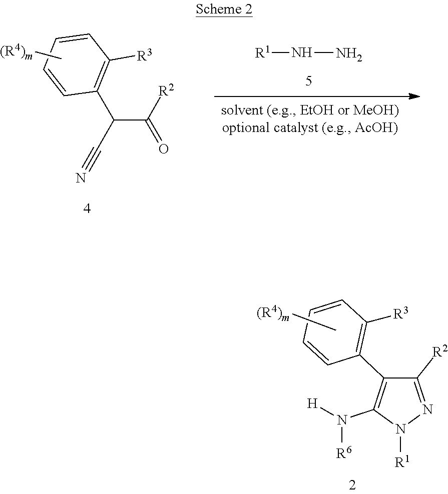

Alternatively, as shown in Scheme 3, 5-aminopyrazoles of Formula 2 can also be prepared by reacting 4-bromo or 4-iodo pyrazoles of Formula 6 with boronic acid compounds of Formula 7 using well-known transition-metal-catalyzed cross-coupling reaction conditions.

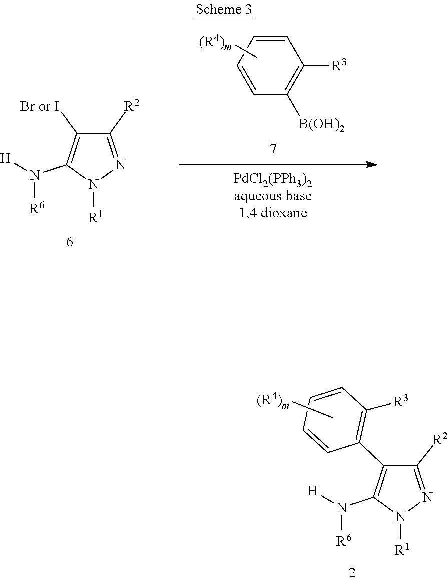

Methods useful for preparing compounds of Formula 6 are known in the art.

Compounds of Formula 1a (i.e. Formula 1 wherein R⁶ is H) can be prepared as shown in Scheme 4. In this method, compounds of Formula 8 are condensed with a hydrazine of Formula 5 (e.g., methylhydrazine or ethylhydrazine) in a solvent such as ethanol or methanol and optionally in the presence of an acid or base catalyst such as acetic acid, piperidine or sodium methoxide, according to general procedures known in the art. For reaction conditions see, PCT Patent Publication WO 2013/116251, Synthesis Example 1, Step C and Example 2, Step B. Also, the method of Scheme 4 using a compound of Formula 8 wherein R^{a} is methyl is illustrated in Example 2, Step C of the present invention.

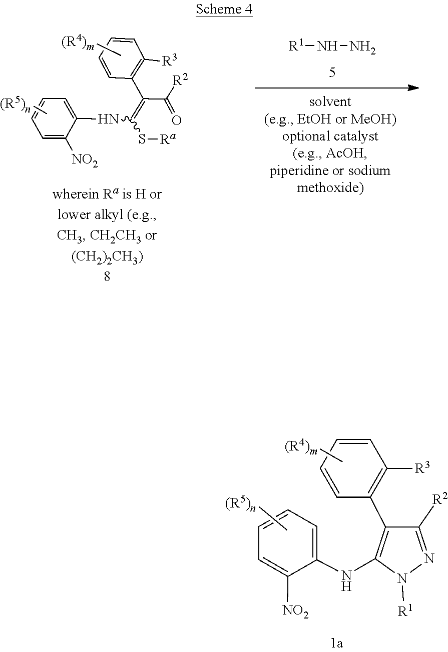

As shown in Scheme 5, compounds of Formula 8 can be prepared by reacting ketene dithioacetal derivatives of Formula 9 with compounds of Formula 10 optionally in the presence of a base, such as sodium hydride or ethylmagnesium chloride, in solvents such as toluene, tetrahydrofuran or dimethoxymethane, at temperatures ranging from about −10° C. to the boiling point of the solvent. For a related reference see, for example, J. Heterocycl. Chem. 1975, 12(1), 139. Methods useful for preparing compounds of Formula 9 are known in the art.

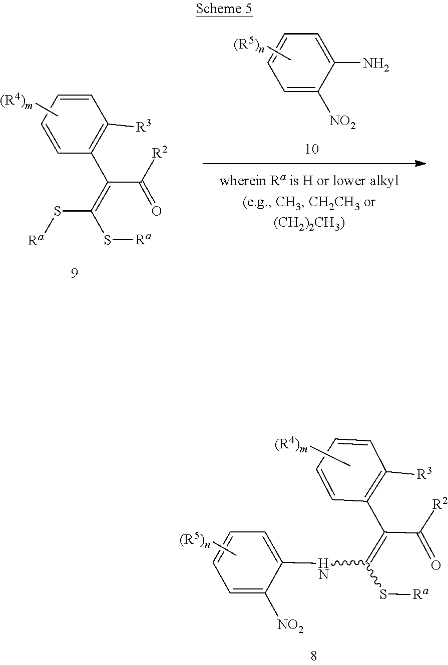

Additionally, as shown in Scheme 6, compounds of Formula 8 wherein R^{a} is lower alkyl (e.g., methyl, ethyl, n-propyl) and Formula 8a (i.e. tautomer of Formula 8 when R^{a} is H) can be prepared via a condensation reaction of isothiocyanate compounds of Formula 11 with carbonyl compounds of Formula 12 to give intermediate compounds of Formula 13, which are salts of the thioamides of Formula 8a. The intermediate compounds of Formula 13 can either be used in situ (as is illustrated in WO 2013/116251, Synthesis Example 1, Step C; and present Example 2, Step C) or isolated (as is illustrated in WO 2013/116251, Example 2, Step A). Bases useful for preparing compounds of Formula 13 include hydrides, alkoxides, hydroxides or carbonates of sodium or potassium, such as sodium hydride, potassium tert-butoxide, sodium ethoxide, potassium hydroxide, sodium hydroxide or potassium carbonate. Amine bases (e.g., triethylamine or N,N-diisopropylethylamine) can also be used to effect the condensation of the compounds of Formulae 11 and 12 to Formula 13. A variety of solvents are useful, such as tetrahydrofuran, diethyl ether, toluene, N,N-dimethylformamide, alcohols (e.g., ethanol), esters (e.g., ethyl acetate or isopropyl acetate), or mixtures thereof. Solvents are chosen for compatibility with the base, as is understood by those skilled in the art. Reaction temperatures can range from −78° C. to the boiling point of the solvent. One useful mixture of base and solvent combination is potassium tert-butoxide or potassium tert-pentoxide in tetrahydrofuran, to which can be added a solution of an isothiocyanate of Formula 11 and a carbonyl compound of Formula 12, which are either combined into one solution, or added separately, preferably by addition of the carbonyl compound followed by addition of the isothiocyanate. Typically this reaction is run at −70 to 0° C. The salt of Formula 13 can be acidified to form the ketothioamide compound of Formula 8a or alkylated with R^{a}X¹ (Formula 14) wherein R^{a} is lower alkyl (e.g., methyl, ethyl, n-propyl) and X¹ is a nucleofuge (i.e. a nucleophilic reaction leaving group such as Br, I, OS(O)₂CH₃) to form the corresponding compound of Formula 8. This general method is known in the chemical literature; see, for example, Zhurnal Organicheskoi Khimii 1982, 18(12), 2501. The method of Scheme 6 to prepare a compound of Formula 8 wherein R^{a} is methyl from an intermediate compound of Formula 13, which is not isolated, is illustrated in PCT Patent Publication WO 2013/116251 Synthesis Example 1, Step C. Also, present Example 2, Step C illustrates the preparation of a compound of Formula 8.

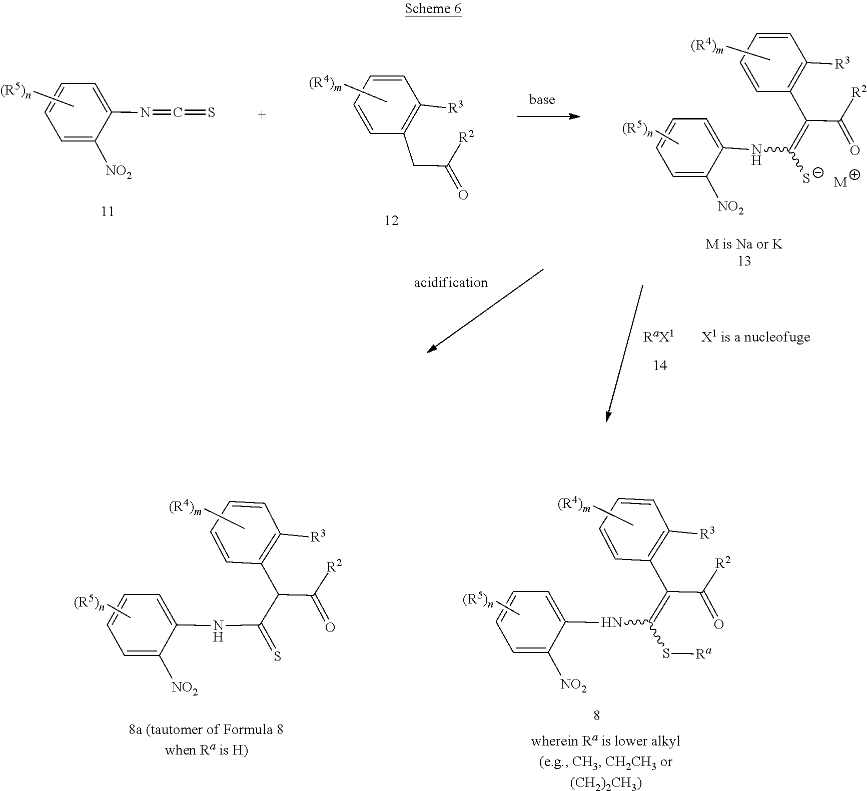

Ketothioamides of Formula 8a can also be prepared by allowing the corresponding ketoamides to react with sulfurizing agents such as Lawesson's reagent or P₂S₅; see, for example, Helv. Chim. Act. 1998, 81(7), 1207.

As shown in Scheme 7, compounds of Formula 1 can also be prepared by reacting 1H-pyrazole compounds of Formula 15 with methylating agents of formula R¹-L² wherein R¹ is methyl or ethyl and L² is a leaving group such as halogen (e.g., Cl, Br, I), sulfonate (e.g., mesylate, triflate or p-toluenesulfonate) or phosphate (e.g., dimethyl phosphate), preferably in the presence of a base such as 1,8-diazabicyclo[5.4.0]undec-7-ene, potassium carbonate or potassium hydroxide, and a solvent such as N,N-dimethylformamide, tetrahydrofuran, toluene or water. General procedures for methylations of this type are well-known in the art and can be readily adapted to prepare compounds of the present invention. Particularly useful methylating agents include diazomethane and iodomethane using general procedures known in the art, such as those described in Canada Journal of Chemistry 1986, 64, 2211-2219 and Heterocycles 2000, 53(12), 2775-2780.

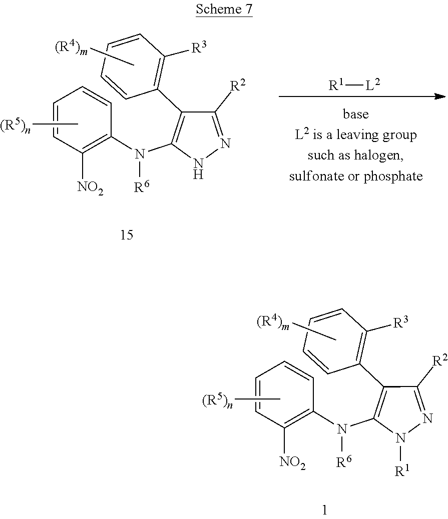

Compounds of Formula 15 can be prepared by condensing compounds of Formula 8 with hydrazine, in a manner analogous to the method of Scheme 4. This method is described in Chemistry of Heterocyclic Compounds 2005, 41(1), 105-110.

In an alternative method, as shown in Scheme 8, compounds of Formula 1 can be prepared by reaction of 4-bromo or 4-iodo pyrazoles of Formula 16 with organometallic compounds of Formula 17 under transition-metal-catalyzed cross-coupling reaction conditions, in the presence of a suitable palladium, copper or nickel catalyst. In this method compounds of Formula 17 are organoboronic acids (e.g., M¹ is B(OH)₂), organoboronic esters (e.g., M¹ is B(—OC(CH₂)₃O—), organotrifluoroborates (e.g., M¹ is BF₃K), organotin reagents (e.g., M¹ is Sn(n-Bu)₃, Sn(Me)₃), Grignard reagents (e.g., M¹ is MgBr or MgCl) or organozinc reagents (e.g., M¹ is ZnBr or ZnCl). Suitable metal catalysts include, but are not limited to: palladium(II) acetate, palladium(II) chloride, tetrakis(triphenylphosphine)-palladium(0), bis(triphenylphosphine)palladium(II) dichloride, dichloro[1,1′-bis(diphenyl-phosphino)ferrocene]palladium(II), bis(triphenylphosphine)dichloronickel(II) and copper(I) salts (e.g., copper(I) iodide, copper(I) bromide, copper(I) chloride, copper(I) cyanide or copper(I) triflate). Optimal conditions will depend on the catalyst used and the counterion attached to the coupling reagent (i.e. M¹), as is understood by one skilled in the art. In some cases the addition of a ligand such as a substituted phosphine or a substituted bisphosphinoalkane promotes reactivity. Also, the presence of a base such as an alkali carbonate, tertiary amine or alkali fluoride may be necessary for some reactions involving organoboron reagents of the Formula 17. For reviews of this type of reaction see: E. Negishi, Handbook of Organopalladium Chemistry for Organic Synthesis, John Wiley and Sons, Inc., New York, 2002; N. Miyaura, Cross-Coupling Reactions: A Practical Guide, Springer, New York, 2002; H. C. Brown et al., Organic Synthesis via Boranes, Vol. 3, Aldrich Chemical Co., Milwaukee, Wis., 2002; Suzuki et al., Chemical Review 1995, 95, 2457-2483 and Molander et al., Accounts of Chemical Research 2007, 40, 275-286. Also, the method of Scheme 8 is illustrated PCT Patent Publications WO 2010/101973 and WO 2012/031061.

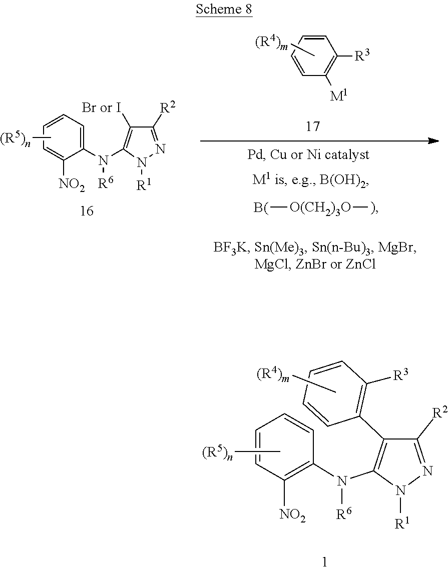

As shown in Scheme 9, pyrazole intermediates of Formula 16 are readily prepared from corresponding pyrazoles of Formula 18 by treatment with a halogenating agent. Suitable halogenating agents for this method include N-bromosuccinimide (NBS), N-iodo-succinimide (NIS), bromine, sodium bromite, thionyl chloride, oxalyl chloride, phenylphosphonic dichloride or phosgene. Particularly useful is N-bromosuccinimide (NBS) and N-iodosuccinimide (NIS). Suitable solvents for this reaction include, for example, N,N-dimethylformamide, N,N-dimethylacetamide, dichloromethane, chloroform, chlorobutane, benzene, xylenes, chlorobenzene, tetrahydrofuran, p-dioxane, acetonitrile, and the like. Optionally, an organic base such as triethylamine, pyridine, N,N-dimethylaniline, and the like can be added. Typical reaction temperatures range from about ambient temperature to 200° C. For representative procedures see Synthesis 2006, 17, 2855-2864; Journal of Medicinal Chemistry 2005, 48, 6843-6854; Journal of Medicinal Chemistry 2007, 50, 3086-3100 and Journal of Medicinal Chemistry 2005, 48, 4420-4431.

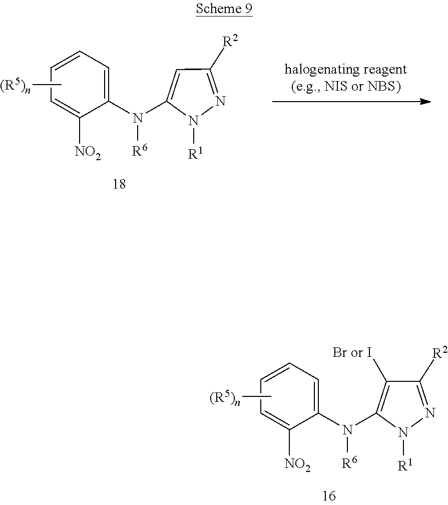

As shown in Scheme 10, compounds of Formula 18 can be prepared from corresponding compounds of Formula 19 by procedures analogous to those used for the method of Scheme 1. Compounds of Formula 19 are commercially available or can be prepared by methods known in the art.

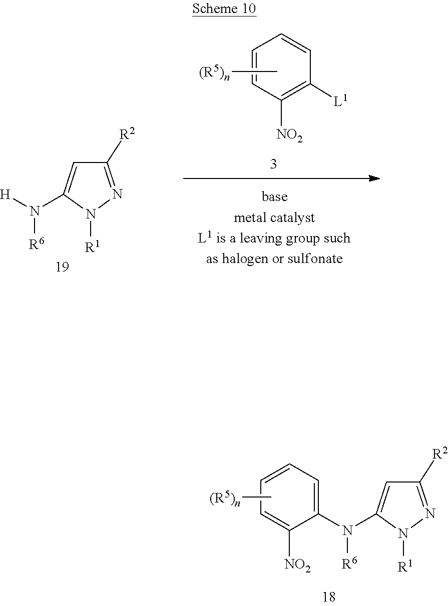

Compounds of Formula 1 and their intermediates described herein can be subjected to various electrophilic, nucleophilic, organometallic, oxidation and reduction reactions to add substituents or modify existing substituents, and thus provide other functionalized compounds of Formula 1. For example, as shown in Scheme 11, compounds of Formula 1b (i.e. Formula 1 wherein (R⁵)ₙ is CH₃) can be prepared by reaction of compounds of Formula 20 wherein L³ leaving group such as a halogen (e.g., Br, I) or sulfonate (e.g., mesylate, triflate, p-toluenesulfonate) with reagents such as 2,4,6-trimethylboroxine or tetramethylstannane in the presence of a catalyst such as [1,1′-bis(diphenylphosphino)-ferrocene]palladium(II) chloride dichloromethane adduct, preferably in the presence of a base such as 1,8-diazabicyclo[5.4.0]undec-7-ene, cesium carbonate or potassium hydroxide and in a solvent such as N,N-dimethylformamide, tetrahydrofuran, 1,4-dioxane, ethanol, toluene or water. The method of Scheme 11 is illustrated PCT Patent Publication WO 2013/192126 Example 4, Step A, and in present Example 4, Step B.

Compounds of Formula 20 can be prepared by methods described in PCT Patent Publications WO 2010/101973 and WO 2012/031061. One skilled in the art will recognize that in some instances preparation of N-protected compounds of Formula 20 prior to functional group interconversions will aid in obtaining the desired products. The choice and use of a suitable N-protection group will be apparent to one skilled in the art; for representative examples see T. W. Greene and P. G. M. Wuts, Protective Groups in Organic Synthesis, 2nd ed.; Wiley: New York, 1991. Also, Step A of present Example 4 illustrates the preparation of an N-Boc protected compound of Formula 20.

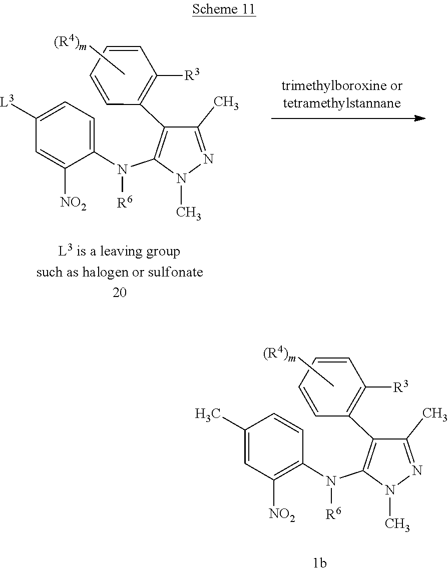

Analogous to the method of Scheme 11, compounds of Formula 20 can be treated with potassium (trifluoromethyl)trimethoxyborate to provide trifluoromethyl analogs of Formula 1b.

In another example, as shown in Scheme 12, compounds of Formula 1 wherein R⁶ is other than H can be prepared from the corresponding compounds of Formula 1 wherein R⁶ is H by reaction with an electrophile comprising R⁶ (i.e. Formula 21). Typically the reaction is done in the presence of a base such as sodium hydride and a polar solvent such as N,N-dimethylformamide. In this context the expression “electrophile comprising R⁶” means a chemical compound capable of transferring an R⁶ moiety to a nucleophile (i.e. the nitrogen atom in Formula 1 when R⁶ is H). Often electrophiles comprising R⁶ have the formula R⁶X² wherein X² is a nucleofuge (i.e. leaving group in nucleophilic reactions). Typical nucleofuges include halide (e.g., Br, Cl, I) or sulfonate (e.g., mesylate, triflate, p-toluenesulfonate). However, some electrophiles comprising R⁶ do not comprise a nucleofuge; an example is sulfur trioxide (SO₃), which after deprotonation (such as by a base of the formula M⁺H⁻ wherein M⁺ is a cation) of the nitrogen atom in Formula 1 when R⁶ is H, can bond to the nitrogen atom as a —SO₃M substituent.

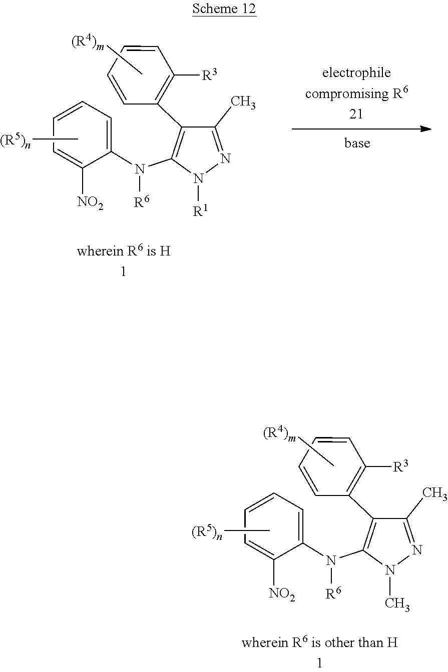

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, T. W. Greene and P. G. M. Wuts, Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “m” means multiplet, “br s” means broad singlet and “dd” means doublet of doublets.

### Example 1

### Preparation of 4-(2-chloro-4-fluorophenyl)-N-(2-fluoro-6-nitrophenyl)-1,3-dimethyl-1H-pyrazol-5-amine (Compound 112)

### Step A: Preparation of α-acetyl-2-chloro-4-fluorobenzeneacetonitrile

A mixture of sodium methoxide solution (30% in methanol, 85 mL, 0.47 mol) in toluene (400 mL) was heated to 120° C. with use of a Dean-Stark trap for the azeotropic removal of methanol. After cooling to 90° C., 2-chloro-4-fluorobenzeneacetonitrile (40.0 g, 0.24 mol) in ethyl acetate (200 mL) was added dropwise to the reaction mixture. The reaction mixture was stirred for 1 h at 90° C., and then hydrochloric acid (1 N, 30 mL) was added. The resulting mixture was extracted with ethyl acetate (3×250 mL) and the combined organic extracts were dried over sodium sulfate, filtered and concentrated under reduced pressure. The resulting material was purified by column chromatography on silica gel (eluting with 3:7 ethyl acetate-petroleum ether) to provide the title compound as a white solid (35 g).

¹H NMR (CDCl₃): δ 7.49 (dd, 1H), 7.24 (dd, 1H), 7.14-7.09 (m, 1H), 5.13 (s, 1H), 2.36 (s, 3H).

### Step B: Preparation of 4-(2-chloro-4-fluorophenyl)-1,3-dimethyl-1H-pyrazol-5-amine

To a mixture of α-acetyl-2-chloro-4-fluorobenzeneacetonitrile (i.e. the product of Step A) (28 g, 0.13 mol) in ethanol (400 mL) was added methylhydrazine sulfate (28.6 g, 0.20 mol) and sodium acetate (21.7 g, 0.27 mol). The reaction mixture was heated at 120° C. for 12 h, and then concentrated under reduced pressure to remove the solvent. The resulting mixture was poured into ice-water (500 mL) and filtered collecting a white solid. The solid was rinsed with water and pentane, then dried to provide the title compound as an off-white solid (24 g).

¹H NMR (CDCl₃): δ 7.45 (dd, 1H), 7.27 (t, 1H), 7.23-7.12 (m, 1H), 4.89 (s, 2H), 3.49 (s, 3H).

### Step C: Preparation of 4-(2-chloro-4-fluorophenyl)-N-(2-fluoro-6-nitrophenyl)-1,3-dimethyl-1H-pyrazol-5-amine

To a mixture of 4-(2-chloro-4-fluorophenyl)-1,3-dimethyl-1H-pyrazol-5-amine (i.e. the product of Step B) (1.2 g, 5.0 mmol) in tetrahydrofuran (40 mL) at 0° C. was added potassium tert-butoxide (1 M in THF, 10 mL, 10 mmol) portion-wise. The reaction mixture was stirred for 1 h at 0° C., and then 1,2-difluoro-3-nitrobenzene (0.85 g, 5.3 mmol) was added drop-wise. After 30 minutes at 0° C., saturated aqueous ammonium chloride was added to the reaction mixture and the resulting mixture was extracted with ethyl acetate (100 mL). The aqueous layer was further extracted with ethyl acetate (2×40 mL) and the combined organic extracts were washed with saturated sodium chloride solution, dried over sodium sulfate, filtered, and concentrated under reduced pressure. The resulting material was purified by column chromatography on silica gel (eluting with 40% ethyl acetate in petroleum ether) to provide the title compound, a compound of the present invention, as a yellow solid (1.1 g).

1H NMR (CDCl₃): δ 8.59 (s, 1H), 7.59 (d, 1H), 7.31 (d, 1H), 7.2 (d, 1H), 7.09 (t, 1H), 7.04-7.01 (m, 1H), 6.82-6.86 (m, 1H), 3.74 (s, 3H), 1.97 (s, 3H).

### Example 2

### Preparation of 3-chloro-4-[5-[(2-fluoro-6-nitrophenyl)amino]-1,3-dimethyl-1H-pyrazol-4-yl]benzonitrile (Compound 113)

Step A: Preparation of 3-chloro-4-(2-oxopropyl)benzonitrile

To a mixture of 4-amino-3-chlorobenzonitrile (50.0 g, 0.33 mol) in diethyl ether (500 mL) at −10° C. was added boron trifluoride diethyl etherate (61 mL, 0.50 mol). The reaction mixture was stirred at −10° C. for 10 minutes, and then tert-butylnitrite (48 mL, 0.4 mol) was added. After 20 minutes at −10° C., the reaction mixture was allowed to warm to room temperature, stirred for 2 h, and then filtered collecting a white solid. The white solid was triturated with diethyl ether and pentane (1:1, 300 mL), filtered and dried to provide the intermediate compound 2-chloro-4-cyanobenzene diazonium tetrafluoroborate salt as an off-white solid (72 g).

To a mixture of 2-chloro-4-cyanobenzene diazonium tetrafluoroborate salt (72 g, 0.33 mol) in dimethylformamide (500 mL) at −10° C. was added isopropenyl acetate (354 mL, 3.2 mol). The reaction mixture was stirred for 20 minutes at −10° C., and then 4-aminomorpholine (1.0 mL) in dimethyl sulfoxide (40 mL) was added. After 1 h, ice-cold water (1000 mL) was added and the resulting mixture was extracted with ethyl acetate (3×250 mL). The combined organic extracts were dried over sodium sulfate, filtered and concentrated under reduced pressure. The resulting material was purified by column chromatography on silica gel (eluting with 1:4 ethyl acetate-petroleum ether) to provide the title compound as a solid (52 g).

¹H NMR (CDCl₃): δ 7.69 (s, 1H), 7.53 (d, 1H), 7.32 (d, 1H), 3.93 (s, 2H), 2.28 (s, 3H).

### Step B: Preparation of 1-fluoro-2-isothiocyanato-3-nitrobenzene

To a mixture of 2-fluoro-6-nitrobenzenamine (1.0 g, 6.4 mmol) in 1,2-dichlorobenzene (10 mL) at 0° C. was added 2 drops of dimethylformamide followed by thiophosgene (1.46 mL, 19 mmol). The reaction mixture was heated at 160° C. for 1 h, cooled to room temperature and concentrated under reduced pressure. The resulting material was purified by column chromatography on silica gel (eluting with 1:9 ethyl acetate-petroleum ether) to provide the title compound as an oil (0.91 g).

¹H NMR (CDCl₃) δ 7.88 (d, 1H), 7.46 (t, 1H), 7.36 (m, 1H).

### Step C: Preparation of 3-chloro-4-[5-[(2-fluoro-6-nitrophenyl)amino]-1,3-dimethyl-1H-pyrazol-4-yl]benzonitrile

To a mixture of 3-chloro-4-(2-oxopropyl)benzonitrile (i.e. the product of Step A) (1.0 g, 5.2 mmol) in tetrahydrofuran (20 mL) at −10° C. was added potassium tert-butoxide (0.7 g, 6.2 mmol). After 30 minutes at −10° C., 1-fluoro-2-isothiocyanato-3-nitrobenzene (i.e. the product of Step B) (0.99 g, 5.0 mmol) in tetrahydrofuran (10 mL) was added to the reaction mixture and stirring was continued for about 15 minutes to provide a reaction mixture containing the intermediate compound 4-[1-[1[(2-chloro-6-nitrophenyl)amino]mercaptomethylene]-2-oxopropyl]-3-chloro-benzonitrile potassium salt, which is the potassium salt of α-acetyl-N-(2-chloro-6-nitrophenyl)-2-chloro-4-cyano-benzeneethane-thioamide. Iodomethane (1.2 mL, 19 mmol) was added to the reaction mixture. After 20 minutes at −10° C., the reaction temperature was brought to 0° C., and acetic acid (5.0 mL) and methylhydrazine (85% in water, 0.5 g, 10 mmol) were added. The reaction mixture was allowed to warm to room temperature, heated at reflux for 2 h, and then poured into ice-cold water (30 mL) and ethyl acetate (20 mL). The organic layer was separated, and the aqueous layer was extracted with ethyl acetate (2×10 mL). The combined organic extracts were washed with saturated sodium chloride solution (20 mL), dried over sodium sulfate, filtered and concentrated under reduced pressure. The resulting material was purified by column chromatography on silica gel (eluting with 2:3 ethyl acetate-petroleum ether) to provide the title compound, a compound of the present invention, as a pale-yellow solid (0.850 g).

1H NMR (CDCl₃) δ 8.71 (d, 1H), 7.85 (d, 1H), 7.64-7.58 (m, 2H), 7.34-7.25 (m, 2H), 6.87-6.81 (m, 1H), 3.75 (s, 3H), 1.99 (s, 3H).

### Example 3

### Preparation of N-(4-bromo-2-fluoro-6-nitrophenyl)-4-(2-chloro-4-fluorophenyl)-1,3-dimethyl-1H-pyrazol-5-amine (Compound 61)

To a mixture of 4-(2-chloro-4-fluorophenyl)-1,3-dimethyl-1H-pyrazol-5-amine (i.e. the product of Example 1, Step B) (0.5 g, 2.1 mmol) in tetrahydrofuran (30 mL) at 0° C. was added potassium tert-butoxide (1 M in THF, 4.2 mL, 4.2 mmol) portion-wise. The reaction mixture was stirred for 1 h at 0° C., and then 5-bromo-1,2-difluoro-3-nitrobenzene (0.54 g, 2.3 mmol) was added drop-wise. After 30 minutes at 0° C., saturated aqueous ammonium chloride was added to the reaction mixture and the resulting mixture was extracted with ethyl acetate (100 mL). The aqueous layer was further extracted with ethyl acetate (2×40 mL) and the combined organic extracts were washed with saturated sodium chloride solution, dried over sodium sulfate, filtered, and concentrated under reduced pressure. The resulting material was purified by column chromatography on silica gel (eluting with 40% ethyl acetate in petroleum ether) to provide the title compound, a compound of the present invention, as a yellow solid (0.45 g).

¹H NMR (CDCl₃): δ 8.69 (br s, 1H), 7.77 (t, 1H), 7.66 (dd, 1H), 7.27 (dd, 2.0 Hz, 1H), 7.09-7.06 (m, 2H), 3.73 (s, 3H), 1.97 (s, 3H).

### Example 4

### Preparation of 4-(2-chloro-4-fluorophenyl)-N-(2-fluoro-4-methyl-6-nitrophenyl)-1,3-dimethyl-1H-pyrazol-5-amine (Compound 93)

### Step A: Preparation of 1,1-Dimethylethyl N-(4-bromo-2-fluoro-6-nitrophenyl)-N-[4-(2-chloro-4-fluorophenyl)-1,3-dimethyl-1H-pyrazol-5-yl]carbamate

To a mixture of N-(4-bromo-2-fluoro-6-nitrophenyl)-4-(2-chloro-4-fluorophenyl)-1,3-dimethyl-1H-pyrazol-5-amine (i.e. the product of Example 3) (1 g, 2.2 mmol) and triethylamine (1.24 mL, 8.9 mmol) in dichloromethane (20 mL) at 0° C. was added di-tert-butyl dicarbonate (1.46 g, 6.7 mmol). The reaction mixture was allowed to warm to room temperature and stirred overnight, and then diluted with water (20 mL) and extracted with dichloromethane (2×20 mL). The combined organic extracts were washed with a saturated sodium chloride solution, dried over sodium sulfate, filtered and concentrated under reduced pressure. The resulting material was purified by column chromatography on silica gel (eluting with 40% ethyl acetate in petroleum ether) to provide the title compound as a yellow solid (750 mg).

¹H NMR (CDCl₃): δ 7.85 (s, 1H), 7.78 (s, 1H), 7.52-7.47 (m, 1H), 7.17-7.19 (m, 1H), 6.97-6.88 (m, 1H), 3.8 (s, 3H), 1.96 (s, 3H), 1.49 (s, 9H).

### Step B: Preparation of 4-(2-chloro-4-fluorophenyl)-N-(2-fluoro-4-methyl-6-nitrophenyl)-1,3-dimethyl-1H-pyrazol-5-amine

A mixture of 1,1-dimethylethyl N-(4-bromo-2-fluoro-6-nitrophenyl)-N-[4-(2-chloro-4-fluorophenyl)-1,3-dimethyl-1H-pyrazol-5-yl]carbamate (i.e. the product of Step A) (600 mg, 1.07 mmol), potassium carbonate (372 mg, 2.7 mmol), dichloro[1,1′-bis(diphenyl-phosphino)ferrocene]palladium(II) dichloromethane complex (1:1) (40 mg, 0.05 mmol) and trimethylboroxine (0.54 mL, 3.9 mmol) in 1,4-dioxane (20 mL) was heated at reflux for 3 h. The reaction mixture was diluted with water (15 mL) and extracted with ethyl acetate (2×10 mL). The combined organic extracts were washed with saturated aqueous sodium chloride solution (3×5 mL), dried over sodium sulfate, filtered and concentrated under reduced pressure. The resulting material was dissolved in dichloromethane and trifluoroacetic acid (3:1; 4 mL) and stirred at room temperature for 16 h. The reaction mixture was concentrated under reduced pressure and the resulting material was dissolved in dichloromethane (5 mL) and washed with a saturated aqueous sodium bicarbonate solution (2 mL). The aqueous layer was further extracted with dichloromethane (3×10 mL). The combined organic extracts were washed with a saturated sodium chloride solution, dried over sodium sulfate, filtered and concentrated under reduced pressure. The resulting material was purified by column chromatography on silica gel (eluting with 40% ethyl acetate in petroleum ether) to provide the title compound, a compound of the present invention, as a yellow solid (210 mg).

¹H NMR (CDCl₃) δ 8.41 (s, 1H), 7.45 (s, 1H), 7.24-7.15 (m, 2H), 7.1-7.01 (m, 2H), 3.72 (s, 3H), 2.15 (s, 3H), 1.95 (s, 3H).

### Example 5

### Alternative preparation of 3-chloro-4-[5-[(2-fluoro-6-nitrophenyl)amino]-1,3-dimethyl-1H-pyrazol-4-yl]benzonitrile (Compound 113)

### Step A: Preparation of 1-methyl-hydrazinecarbonitrile

A solution of cyanogen bromide (13.5 g, 127.5 mmol) and dichloromethane (250 mL) was cooled to 0° C., and then a mixture of methyl hydrazine (85% aqueous solution, 6.0 g, 127.5 mmol), sodium carbonate (7.5 g, 63.9 mmol) and water (60 mL) was added dropwise with vigorous stirring. After visible signs of gas evolution stopped, the aqueous layer was separated and extracted with dichloromethane (3×). The combined organic layers were dried over magnesium sulphate, filtered and the filtrate was concentrated under reduced pressure to provide the title compound as an oil (6.0 g).

### Step B: Preparation of 4-(5-amino-1,3-dimethyl-1H-pyrazol-4-yl)-3-chloro-benzonitrile

A mixture of 3-chloro-4-(2-oxopropyl)benzonitrile (13.7 g, 71.4 mmol) and 1-methylhydrazinecarbonitrile (i.e. the product of Step A) (6.0 g, 86 mmol) was heated at 60° C. with stirring. After 48 h, the reaction mixture was dissolved in dichloromethane (100 mL) and water (100 mL), the layers ware separated and the aqueous layer was extracted with dichloromethane (3×). The combined organic layers were dried over magnesium sulphate, filtered and the filtrate was concentrated under reduced pressure. The resulting material was purified by column chromatography on silica gel (eluting with 60% ethyl acetate in petroleum ether) to provide the title compound as a light-yellow solid (8.1 g).

LCMS: 247 (M+1)

### Step C: Preparation of 3-chloro-4-[5-[(2-fluoro-6-nitrophenyl)amino]-1,3-dimethyl-1H-pyrazol-4-yl]benzonitrile

To a mixture of 4-(5-amino-1,3-dimethyl-1H-pyrazol-4-yl)-3-chlorobenzonitrile (i.e. the product of Step B) (1.2 g, 4.8 mol) in tetrahydrofuran (40 mL) at 0° C. was added potassium tert-butoxide (9.7 mL, 1 M in tetrahydrofuran) dropwise. The reaction mixture was stirred at 0° C. for 1 h, and then 1,2-difluoro-3-nitrobenzene (0.85 g, 5.3 mmol) was added dropwise and stirring was continued for an additional 30 minutes at 0° C. The reaction mixture was diluted with saturated aqueous ammonium chloride and ethyl acetate (100 mL), and the layers were separated. The aqueous layer was extracted with ethyl acetate (40 mL×2), and the combined organic extracts were washed with saturated aqueous sodium chloride solution, dried over magnesium sulphate, filtered and the filtrate was concentrated under reduced pressure. The resulting material was purified by column chromatography on silica gel (eluting with 40% ethyl acetate in petroleum ether) to provide a yellow solid. The yellow sold was crystallized from ethanol to provide the title compound, a compound of the present invention, as a light-yellow solid (560 mg).

¹H NMR (CDCl₃) δ 8.71 (d, 1H), 7.85 (d, 1H), 7.63-7.58 (m, 2H), 7.33-7.25 (m, 2H), 6.86-6.82 (m, 1H), 3.75 (s, 3H), 1.99 (s, 3H).

LCMS: 386 (M+1).

By the procedures described herein together with methods known in the art, the compounds disclosed in the Tables that follow can be prepared. The following abbreviations are used in the Tables which follow: Me means methyl, MeO means methoxy, EtO means ethoxy, and CN means cyano.

TABLE 1

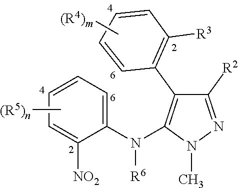

R² is CH₃, R³ is Cl and (R⁴)ₘ is 4-F.
(R⁵)ₙ
6-F
4,6-di-F
4-Cl, 6-F
4-Br, 6-F
4-I, 6-F
4-Me, 6-F
4-MeO, 6-F
4-EtO, 6-F
6-Cl
4,6-di-Cl
4-F, 6-Cl
4-Br, 6-Cl
4-I, 6-Cl
4-Me, 6-Cl
4-MeO, 6-Cl
4-EtO, 6-Cl
6-Br
4,6-di-Br
4-F, 6-Br
4-Cl, 6-Br
4-I, 6-Br
4-Me, 6-Br
4-MeO, 6-Br
4-EtO, 6-Br
6-I
4,6-di-I
4-F, 6-I
4-Cl, 6-I
4-Br, 6-I
4-Me, 6-I
4-MeO, 6-I
4-EtO, 6-I
4-Me
4-MeO
4-EtO
6-Me
6-MeO
6-EtO

The present disclosure also includes Tables 1A through 46A, each of which is constructed the same as Table 1 above, except that the row heading in Table 1 (i.e. “R² is CH₃, R³ is Cl and (R⁴)ₘ is 4-F” is replaced with the respective row headings shown below.

| Table | Row Heading |
|---|---|
| 1A | R² is CH₃, R³ is F and (R⁴)ₘ is 4-F. |
| 2A | R² is CH₃, R³ is Br and (R⁴)ₘ is 4-F. |
| 3A | R² is CH₃, R³ is Me and (R⁴)ₘ is 4-F. |
| 4A | R² is CH₃, R³ is Cl and (R⁴)ₘ is 4-MeO. |
| 5A | R² is CH₃, R³ is F and (R⁴)ₘ is 4-MeO. |
| 6A | R² is CH₃, R³ is Br and (R⁴)ₘ is 4-MeO. |
| 7A | R² is CH₃, R³ is Me and (R⁴)ₘ is 4-MeO. |
| 8A | R² is CH₃, R³ is Cl and (R⁴)ₘ is 4-CN. |
| 9A | R² is CH₃, R³ is F and (R⁴)ₘ is 4-CN. |
| 10A | R² is CH₃, R³ is Br and (R⁴)ₘ is 4-CN |
| 11A | R² is CH₃, R³ is Me and (R⁴)ₘ is 4-CN. |
| 12A | R² is CH₃, R³ is Cl and (R⁴)ₘ is 4,6-di-F. |
| 13A | R² is CH₃, R³ is F and (R⁴)ₘ is 4,6-di-F. |
| 14A | R² is CH₃, R³ is Br and (R⁴)ₘ is 4,6-di-F. |
| 15A | R² is CH₃, R³ is Me and (R⁴)ₘ is 4,6-di-F. |
| 16A | R² is CH₃, R³ is Cl and (R⁴)ₘ is 4-Cl, 6-F. |
| 17A | R² is CH₃, R³ is F and (R⁴)ₘ is 4-Cl, 6-F. |
| 18A | R² is CH₃, R³ is Br and (R⁴)ₘ is 4-Cl, 6-F. |
| 19A | R² is CH₃, R³ is Me and (R⁴)ₘ is 4-Cl, 6-F. |
| 20A | R² is CH₃, R³ is Cl and (R⁴)ₘ is 4-MeO, 6-F. |
| 21A | R² is CH₃, R³ is F and (R⁴)ₘ is 4-MeO, 6-F. |
| 22A | R² is CH₃, R³ is Br and (R⁴)ₘ is 4-MeO, 6-F. |
| 23A | R² is CH₃, R³ is Me and (R⁴)ₘ is 4-MeO, 6-F. |
| 24A | R² is Et, R³ is F and (R⁴)ₘ is 4-F. |
| 25A | R² is Et, R³ is Br and (R⁴)ₘ is 4-F. |
| 26A | R² is Et, R³ is Me and (R⁴)ₘ is 4-F. |
| 27A | R² is Et, R³ is Cl and (R⁴)ₘ is 4-MeO. |
| 28A | R² is Et, R³ is F and (R⁴)ₘ is 4-MeO. |
| 29A | R² is Et, R³ is Br and (R⁴)ₘ is 4-MeO. |
| 30A | R² is Et, R³ is Me and (R⁴)ₘ is 4-MeO. |
| 31A | R² is Et, R³ is Cl and (R⁴)ₘ is 4-CN. |
| 32A | R² is Et, R³ is F and (R⁴)ₘ is 4-CN. |
| 33A | R² is Et, R³ is Br and (R⁴)ₘ is 4-CN |
| 34A | R² is Et, R³ is Me and (R⁴)ₘ is 4-CN. |
| 35A | R² is Et, R³ is Cl and (R⁴)ₘ is 4,6-di-F. |
| 36A | R² is Et, R³ is F and (R⁴)ₘ is 4,6-di-F. |
| 37A | R² is Et, R³ is Br and (R⁴)ₘ is 4,6-di-F. |
| 38A | R² is Et, R³ is Me and (R⁴)ₘ is 4,6-di-F. |
| 39A | R² is Et, R³ is Cl and (R⁴)ₘ is 4-Cl, 6-F. |
| 40A | R² is Et, R³ is F and (R⁴)ₘ is 4-Cl, 6-F. |
| 41A | R² is Et, R³ is Br and (R⁴)ₘ is 4-Cl, 6-F. |
| 42A | R² is Et, R³ is Me and (R⁴)ₘ is 4-Cl, 6-F. |
| 43A | R² is Et, R³ is Cl and (R⁴)ₘ is 4-MeO, 6-F. |
| 44A | R² is Et, R³ is F and (R⁴)ₘ is 4-MeO, 6-F. |
| 45A | R² is Et, R³ is Br and (R⁴)ₘ is 4-MeO, 6-F. |
| 46A | R² is Et, R³ is Me and (R⁴)ₘ is 4-MeO, 6-F. |

TABLE 2

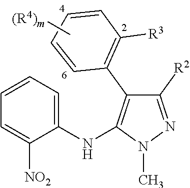

| R² is CH₃ and R³ is Cl. | R² is CH₃, R³ is Cl and (R⁴)ₘ is 4-F. |
|---|---|
| (R⁴)ₘ | (R⁴)ₘ |
| 6-F | 4-I, 6-Br |
| 4,6-di-F | 4-Me, 6-Br |
| 4-Cl, 6-F | 4-MeO, 6-Br |
| 4-Br, 6-F | 4-EtO, 6-Br |
| 4-I, 6-F | 4-CN, 6-Br |
| 4-Me, 6-F | 6-I |
| 4-MeO, 6-F | 4,6-di-I |
| 4-EtO, 6-F | 4-F, 6-I |
| 4-CN, 6-F | 4-Cl, 6-I |
| 6-Cl | 4-Br, 6-I |
| 4,6-di-Cl | 4-Me, 6-I |
| 4-F, 6-Cl | 4-MeO, 6-I |
| 4-Br, 6-Cl | 4-EtO, 6-I |
| 4-I, 6-Cl | 4-CN, 6-I |
| 4-Me, 6-Cl | 4-Me |
| 4-MeO, 6-Cl | 4-MeO |
| 4-EtO, 6-Cl | 4-EtO |
| 4-CN, 6-Cl | 4-CN |
| 6-Br | 6-Me |
| 4,6-di-Br | 6-MeO |
| 4-F, 6-Br | 6-EtO |
| 4-Cl, 6-Br | 6-CN |

### Formulation/Utility

A compound of Formula 1 of this invention (including N-oxides and salts thereof), or a mixture (i.e. composition) comprising the compound with at least one additional fungicidal compound as described in the Summary of the Invention, will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

The mixtures of component (a) (i.e. at least one compound of Formula 1, N-oxides, or salts thereof) with component (b) (e.g., selected from (b1) to (b54) and salts thereof as described above) and/or one or more other biologically active compound or agent (i.e. insecticides, other fungicides, nematocides, acaricides, herbicides and other biological agents) can be formulated in a number of ways, including:
(i) component (a), component (b) and/or one or more other biologically active compounds or agents can be formulated separately and applied separately or applied simultaneously in an appropriate weight ratio, e.g., as a tank mix; or
(ii) component (a), component (b) and/or one or more other biologically active compounds or agents can be formulated together in the proper weight ratio.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Of note is a composition embodiment wherein granules of a solid composition comprising a compound of Formula 1 (or an N-oxide or salt thereof) is mixed with granules of a solid composition comprising component (b). These mixtures can be further mixed with granules comprising additional agricultural protectants. Alternatively, two or more agricultural protectants (e.g., a component (a) (Formula 1) compound, a component (b) compound, an agricultural protectant other than component (a) or (b)) can be combined in the solid composition of one set of granules, which is then mixed with one or more sets of granules of solid compositions comprising one or more additional agricultural protectants. These granule mixtures can be in accordance with the general granule mixture disclosure of PCT Patent Publication WO 94/24861 or more preferably the homogeneous granule mixture teaching of U.S. Pat. No. 6,022,552.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders. | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyl peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquatemary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids. Such formulation auxiliaries and additives may control: pH (buffers), foaming during processing (antifoams such polyorganosiloxanes (e.g., Rhodorsil® 416)), sedimentation of active ingredients (suspending agents), viscosity (thixotropic thickeners), in-container microbial growth (antimicrobials), product freezing (antifreezes), color (dyes/pigment dispersions (e.g., Pro-lzed® Colorant Red)), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pp 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

One embodiment of the present invention relates to a method for controlling fungal pathogens, comprising diluting the fungicidal composition of the present invention (a compound of Formula 1 formulated with surfactants, solid diluents and liquid diluents or a formulated mixture of a compound of Formula 1 and at least one other fungicide) with water, and optionally adding an adjuvant to form a diluted composition, and contacting the fungal pathogen or its environment with an effective amount of said diluted composition.

Although a spray composition formed by diluting with water a sufficient concentration of the present fungicidal composition can provide sufficient efficacy for controlling fungal pathogens, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be anionic or nonionic surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

The amount of adjuvants added to spray mixtures is generally in the range of about 2.5% to 0.1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

One method of seed treatment is by spraying or dusting the seed with a compound of the invention (i.e. as a formulated composition) before sowing the seeds. Compositions formulated for seed treatment generally comprise a film former or adhesive agent. Therefore, typically a seed coating composition of the present invention comprises a biologically effective amount of a compound of Formula 1 and a film former or adhesive agent. Seeds can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be constructed as merely illustrative, and not limiting of the disclosure in any way whatsoever.

### Example A

High Strength Concentrate

| | |
|---|---|
| Compound 60 | 98.5% |
| silica aerogel | 0.5% |
| synthetic amorphous fine silica | 1.0% |

Wettable Powder

| | |
|---|---|
| Compound 68 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 72 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 93 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 112 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 118 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 60 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| Compound 68 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

### Example I

Suspension Concentrate

| | |
|---|---|
| Compound 72 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| Compound 93 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| Compound 112 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| Compound 118 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Water-soluble and water-dispersible formulations are typically diluted with water to form aqueous compositions before application. Aqueous compositions for direct applications to the plant or portion thereof (e.g., spray tank compositions) typically contain at least about 1 ppm or more (e.g., from 1 ppm to 100 ppm) of the compound(s) of this invention.

Seed is normally treated at a rate of from about 0.001 g (more typically about 0.1 g) to about 10 g per kilogram of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Ascomycota, Basidiomycota, Zygomycota phyla, and the fungal-like Comycata class. They are effective in controlling a broad spectrum of plant diseases, particularly foliar pathogens of ornamental, turf, vegetable, field, cereal, and fruit crops. These pathogens include but are not limited to those listed in Table 1-1. For Ascomycetes and Basidiomycetes, names for both the sexual/teleomorph/perfect stage as well as names for the asexual/anamorph/imperfect stage (in parentheses) are listed where known. Synonymous names for pathogens are indicated by an equal sign. For example, the sexual/teleomorph/perfect stage name Phaeosphaeria nodorum is followed by the corresponding asexual/anamorph/imperfect stage name Stagnospora nodorum and the synonymous older name Septoria nodorum.

TABLE 1-1
Ascomycetes in the order Pleosporales including Altemaria solani, A. alternata and A. brassicae,
Guignardia bidwellii, Venturia inaequalis, Pyrenophora tritici-repentis (Dreschlera tritici-repentis =
Helminthosporium tritici-repentis) and Pyrenophora teres (Dreschlera teres = Helminthosporium
teres), Corynespora cassiicola, Phaeosphaeria nodorum (Stagonospora nodorum = Septoria
nodorum), Cochliobolus carbonum and C. heterostrophus, Leptosphaeria biglobosa and L.
maculans;
Ascomycetes in the order Mycosphaerellales including Mycosphaerella graminicola (Zymoseptoria
tritici = Septoria tritici), M. berkeleyi (Cercosporidium personatum), M. arachidis (Cercospora
arachidicola), Passalora sojina (Cercospora sojina), Cercospora zeae-maydis and C. beticola;
Ascomycetes in the order Erysiphales (the powdery mildews) such as Blumeria graminis f. sp. tritici
and Blumeria graminis f. sp. hordei, Erysiphe polygoni, E. necator (=Uncinula necator),
Podosphaera fuliginea (=Sphaerotheca fuliginea), and Podosphaera leucotricha (=Sphaerotheca
fuliginea);
Ascomycetes in the order Helotiales such as Botryotinia fuckeliana (Botrytis cinerea), Oculimacula
yallundae (=Tapesia yallundae; anamorph Helgardia herpotrichoides = Pseudocercosporella
herpetrichoides), Monilinia fructicola, Sclerotinia sclerotiorum, Sclerotinia minor, and Sclerotinia
homoeocarpa;
Ascomycetes in the order Hypocreales such as Giberella zeae (Fusarium graminearum), G.
monoliformis (Fusarium moniliforme), Fusarium solani and Verticillium dahliae;
Ascomycetes in the order Eurotiales such as Aspergillus flavus and A. parasiticus;
Ascomycetes in the order Diaporthales such as Cryptosphorella viticola (=Phomopsis viticola),
Phomopsis longicolla, and Diaporthe phaseolorum;
Other Ascomycete pathogens including Magnaporthe grisea, Gaeumannomyces graminis,
Rhynchosporium secalis, and anthracnose pathogens such as Glomerella acutata (Colletotrichum
acutatum), G. graminicola (C. graminicola) and G. lagenaria (C. orbiculare);
Basidiomycetes in the order Urediniales (the rusts) including Puccinia recondita, P. striiformis,
Puccinia hordei, P. graminis and P. arachidis), Hemileia vastatrix and Phakopsora pachyrhizi;
Basidiomycetes in the order Ceratobasidiales such as Thanatophorum cucumeris (Rhizoctonia solani)
and Ceratobasidium oryzae-sativae (Rhizoctonia oryzae);
Basidiomycetes in the order Polyporales such as Athelia rolfsii (Sclerotium rolfsii);
Basidiomycetes in the order Ustilaginales such as Ustilago maydis;
Zygomycetes in the order Mucorales such as Rhizopus stolonifer;
Oomycetes in the order Pythiales, including Phytophthora infestans, P. megasperma, P. parasitica,
P. sojae, P. cinnamomi and P. capsici, and Pythium pathogens such as Pythium aphanidermatum, P.
graminicola, P. irregulare, P. ultimum and P. dissoticum;
Oomycetes in the order Peronosporales such as Plasmopara viticola, P. halstedii, Peronospora
hyoscyami (=Peronospora tabacina), P. manshurica, Hyaloperonospora parasitica (=Peronospora
parasitica), Pseudoperonospora cubensis and Bremia lactucae;
and other genera and species closely related to all of the above pathogens.

In addition to their fungicidal activity, the compositions or combinations also have activity against bacteria such as Erwinia amylovora, Xanthomonas campestris, Pseudomonas syringae, and other related species. By controlling harmful microorganisms, the compounds of the invention are useful for improving (i.e. increasing) the ratio of beneficial to harmful microorganisms in contact with crop plants or their propagules (e.g., seeds, corms, bulbs, tubers, cuttings) or in the agronomic environment of the crop plants or their propagules.

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance.

Treatment of genetically modified plants and seeds with compounds of the invention may result in super-additive or enhanced effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds and compositions of this invention are useful in seed treatments for protecting seeds from plant diseases. In the context of the present disclosure and claims, treating a seed means contacting the seed with a biologically effective amount of a compound of this invention, which is typically formulated as a composition of the invention. This seed treatment protects the seed from soil-borne disease pathogens and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate.

Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

Compounds of this invention and their compositions, both alone and in combination with other fungicides, nematicides and insecticides, are particularly useful in seed treatment for crops including, but not limited to, maize or corn, soybeans, cotton, cereal (e.g., wheat, oats, barley, rye and rice), potatoes, vegetables and oilseed rape.

Furthermore, the compounds of this invention are useful in treating postharvest diseases of fruits and vegetables caused by fungi and bacteria. These infections can occur before, during and after harvest. For example, infections can occur before harvest and then remain dormant until some point during ripening (e.g., host begins tissue changes in such a way that infection can progress); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g., fruits, seeds, foliage, stems, bulbs, tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example, mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

The compounds can also be applied using an unmanned aerial vehicle (UAV) for the dispension of the compositions disclosed herein over a planted area. In some embodiments the planted area is a crop-containing area. In some embodiments, the crop is selected from a monocot or dicot. In some embodiments, the crop is selected form rice, corn, barley, sobean, wheat, vegetable, tobacco, tea tree, fruit tree and sugar cane. In some embodiments, the compositions disclosed herein are formulated for spraying at an ultra-low volume. Products applied by drones may use water or oil as the spray carrier. Typical spray volume (including product) used for drone applications globally. 5.0 liters/ha-100 liters/ha (approximately 0.5-10 gpa). This includes the range of ultra low spray volume (ULV) to low spray volume (LV). Although not common there may be situations where even lower spray volumes could be used as low as 1.0 liter/ha (0.1 gpa).

Suitable rates of application (e.g., fungicidally effective amounts) of component (a) (i.e. at least one compound selected from compounds of Formula 1, N-oxides and salts thereof) as well as suitable rates of application (e.g., biologically effective amounts, fungicidally effective amounts or insecticidally effective amounts) for the mixtures and compositions comprising component (a) according to this invention can be influenced by factors such as the plant diseases to be controlled, the plant species to be protected, the population structure of the pathogen to be controlled, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.001 g (more typically about 0.1 g) to about 10 g per kilogram of seed. One skilled in the art can easily determine through simple experimentation the application rates of component (a), and mixtures and compositions thereof, containing particular combinations of active ingredients according to this invention needed to provide the desired spectrum of plant protection and control of plant diseases and optionally other plant pests.

Compounds and compositions of the present invention may also be useful for increasing vigor of a crop plant. This method comprises contacting the crop plant (e.g., foliage, flowers, fruit or roots) or the seed from which the crop plant is grown with a compositions comprising a compound of Formula 1 in amount sufficient to achieve the desired plant vigor effect (i.e. biologically effective amount). Typically the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds and compositions of the present invention may increase the vigor of treated plants compared to untreated plants by preventing and/or curing plant diseases caused by fungal plant pathogens in the environment of the plants. In the absence of such control of plant diseases, the diseases reduce plant vigor by consuming plant tissues or sap, or transmitting plant pathogens such as viruses. Even in the absence of fungal plant pathogens, the compounds of the invention may increase plant vigor by modifying metabolism of plants. Generally, the vigor of a crop plant will be most significantly increased by treating the plant with a compound of the invention if the plant is grown in a nonideal environment, i.e. an environment comprising one or more aspects adverse to the plant achieving the full genetic potential it would exhibit in an ideal environment.

Of note is a method for increasing vigor of a crop plant wherein the crop plant is grown in an environment comprising plant diseases caused by fungal plant pathogens. Also of note is a method for increasing vigor of a crop plant wherein the crop plant is grown in an environment not comprising plant diseases caused by fungal plant pathogens. Also of note is a method for increasing vigor of a crop plant wherein the crop plant is grown in an environment comprising an amount of moisture less than ideal for supporting growth of the crop plant.

Compounds and compositions of this invention can also be mixed with one or more other biologically active compounds or agents including fungicides, insecticides, nematicides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

As mentioned in the Summary of the Invention, one aspect of the present invention is a fungicidal composition comprising (i.e. a mixture or combination of) a compound of Formula 1, an N-oxide, or a salt thereof (i.e. component a), and at least one other fungicide (i.e. component b). Of note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a fungicidally effective amount of at least one additional fungicidal active ingredient having a similar spectrum of control but a different site of action.

Examples of component (b) fungicides include acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, captafol, captan, carbendazim, carboxin, carpropamid, chloroneb, chlorothalonil, chlozolinate, clotrimazole, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, dipymetitrone, econazole, edifenphos, enoxastrobin (also known as enestroburin), epoxiconazole, etaconazole, ethaboxam, ethirimol, etridiazole, famoxadone, fenamidone, fenarimol, fenaminstrobin, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, flometoquin, florylpicoxamid, fluazinam, fludioxonil, flufenoxystrobin, fluindapyr, flumorph, fluopicolide, fluopimomide, fluopyram, flouroimide, fluoxastrobin, fluquinconazole, flusilazole, flusulfamide, flutianil, flutolanil, flutriafol, fluxapyroxad, folpet, fthalide, fuberidazole, furalaxyl, furametpyr, guazatine, hexaconazole, hymexazole, imazalil, imibenconazole, iminoctadine albesilate, iminoctadine triacetate, iodocarb, ipconazole, ipfentrifluconazole, iprobenfos, iprodione, iprovalicarb, isoconazole, isofetamid, isoprothiolane, isoflucypram, isopyrazam, isotianil, kasugamycin, kresoxim-methyl, mancozeb, mandepropamid, mandestrobin, maneb, mepanipyrim, mepronil, meptyldinocap, metalaxyl (including metalaxyl-M/mefenoxam), mefentrifluconazole, metconazole, methasulfocarb, metiram, metominostrobin, metrafenone, miconazole, myclobutanil, naftifine, neo-asozin, nuarimol, octhilinone, ofurace, orysastrobin, oxadixyl, oxathiapiprolin, oxolinic acid, oxpoconazole, oxycarboxin, oxytetracycline, pefurazoate, penconazole, pencycuron, penflufen, penthiopyrad, phosphorous acid (including salts thereof, e.g., fosetyl-aluminum), picarbutrazox, picoxystrobin, piperalin, polyoxin, probenazole, prochloraz, procymidone, propamacarb, propiconazole, propineb, proquinazid, prothiocarb, prothioconazole, pyraclostrobin, pyrametostrobin, pyraoxystrobin, pyrazophos, pyribencarb, pyributicarb, pyrifenox, pyrimethanil, pyriofenone, pyrisoxazole, pyroquilon, pyrrolnitrin, quinconazole, quinofumelin (Registry Number 861647-84-9) quinomethionate, quinoxyfen, quintozene, sedaxane, silthiofam, simeconazole, spiroxamine, streptomycin, sulfur, tebuconazole, tebufloquin, teclofthalam, tecnazene, terbinafine, tetraconazole, thiabendazole, thifluzamide, thiophanate, thiophanate-methyl, thiram, tiadinil, tolclofos-methyl, tolnifanide, tolprocarb, tolyfluanid, triadimefon, triadimenol, triarimol, triticonazole, triazoxide, tribasic copper sulfate, tricyclazole, triclopyricarb, tridemorph, trifloxystrobin, triflumizole, triforine, trimorphamide, uniconazole, uniconazole-P, validamycin, valifenalate (also known as valiphenal), vinclozolin, zineb, ziram, zoxamide, N-[2-(1S,2R)-[1,1′-bicyclopropyl]-2-ylphenyl]-3-(difluoromethyl)-1-methyl-1H-pyrazole-4-carboxamide, α-(1-chloro-cyclopropyl)-α-[2-(2,2-dichlorocyclopropyl)ethyl]-1H-1,2,4-triazole-1-ethanol, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, rel-1-[[(2R,3S)-3-(2-chlorophenyl)-2-(2,4-difluorophenyl)-2-oxiranyl]methyl]-5-(2-propen-1-ylthio)-1H-1,2,4-triazole, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-1-[4-[[3-(4-chlorophenyl)-2-propyn-1-yl]oxy]-3-methoxyphenyl]ethyl]-3-methyl-2-[(ethylsulfonyl)amino]butanamide, N-[4-[4-chloro-3-(trifluoromethyl)phenoxy]-2,5-dimethylphenyl]-N-ethyl-N-methylmethanimidamide, N-[[(cyclopropylmethoxy)amino][6-(difluoromethoxy)-2,3-difluorophenyl]methylene]benzeneacetamide, N-[2-(2,4-dichloro-phenyl)-2-methoxy-1-methylethyl]-3-(difluoromethyl)-1-methyl-1H-pyrazole-4-carboxamide, N-(3′,4′-difluoro[1,1′-biphenyl]-2-yl)-3-(trifluoromethyl)-2-pyrazinecarbox-amide, 3-(difluoromethyl)-N-(2,3-dihydro-1,1,3-trimethyl-1H-inden-4-yl)-1-methyl-1H-pyrazole-4-carboxamide, 5,8-difluoro-N-[2-[3-methoxy-4-[[4-(trifluoromethyl)-2-pyridinyl]-oxy]phenyl]ethyl]-4-quinazolinamine, 1-[4-[4-[5R-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone, 4-fluorophenyl N-[1-[[[1-(4-cyanophenyl)ethyl]sulfonyl]methyl]-propyl]carbamate, 5-fluoro-2-[(4-fluorophenyl)methoxy]-4-pyrimidinamine, α-(methoxy-imino)-N-methyl-2-[[[1-[3-(trifluoromethyl)phenyl]ethoxy]imino]methyl]benzeneacetamide, and [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]carbonyl]-3-pyridinyl]oxy]methyl 2-methyl-propanoate. Therefore of note is a fungicidal composition comprising as component (a) a compound of Formula 1 (or an N-oxide or salt thereof) and as component (b) at least one fungicide selected from the preceding list.

Of particular note are combinations of compounds of Formula 1 (or an N-oxide or salt thereof) (i.e. Component (a) in compositions) with component (b) compounds selected from aminopyrifen (Registry Number 1531626-08-0), azoxystrobin, benzovindiflupyr, bixafen, captan, carpropamid, chlorothalonil, copper hydroxide, copper oxychloride, copper sulfate, cymoxanil, cyproconazole, cyprodinil, dichlobentiazox (Registry Number 957144-77-3), diethofencarb, difenoconazole, dimethomorph, epoxiconazole, ethaboxam, fenarimol, fenhexamid, fluazinam, fludioxonil, fluindapyr, fluopyram, flusilazole, flutianil, flutriafol, fluxapyroxad, folpet, ipflufenoquin (Registry Number 1314008-27-9), iprodione, isofetamid, isoflucypram, isopyrazam, kresoxim-methyl, mancozeb, mandestrobin, meptyldinocap, metalaxyl (including metalaxyl-M/mefenoxam), mefentrifluconazole, metconazole, metrafenone, metyltetraprole (Registry Number 1472649-01-6), myclobutanil, oxathiapiprolin, penflufen, penthiopyrad, phosphorous acid (including salts thereof, e.g., fosetyl-aluminum), picoxystrobin, propiconazole, proquinazid, prothioconazole, pyridachlometyl (Registry Number 1358061-55-8), pyraclostrobin, pyrapropoyne (Registry Number 1803108-03-3), pyrimethanil, sedaxane spiroxamine, sulfur, tebuconazole, thiophanate-methyl, trifloxystrobin, zoxamide, α-(1-chlorocyclopropyl)-α-[2-(2,2-dichloro-cyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, N-[2-(2,4-dichlorophenyl)-2-methoxy-1-methylethyl]-3-(difluoromethyl)-1-methyl-1H-pyrazole-4-carboxamide, 3-(difluoromethyl)-N-(2,3-dihydro-1,1,3-trimethyl-1H-inden-4-yl)-1-methyl-1H-pyrazole-4-carboxamide, 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, 1,1-di-methylethyl N-[6-[[[[(1-methyl-1H-tetrazol-5-yl)phenylmethylene]amino]oxy]methyl]-2-pyridinyl]carbamate, dipymetitrone, 5-fluoro-2-[(4-fluorophenyl)methoxy]-4-pyrimidin-amine, 5-fluoro-2-[(4-methylphenyl)methoxy]-4-pyrimidinamine, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-ox-iranyl]methyl]-5-(2-propen-1-ylthio)-1H-1,2,4-triazole (i.e. as Component (b) in compostions).

Of particular note are combinations of compounds of Formula 1 (or an N-oxide or salt thereof) (i.e. Component (a) in compositions) with component (b) compounds selected from aminopyrifen (Registry Number 1531626-08-0), azoxystrobin, benzovindiflupyr, bixafen, captan, carpropamid, chlorothalonil, copper hydroxide, copper oxychloride, copper sulfate, cymoxanil, cyproconazole, cyprodinil, dichlobentiazox (Registry Number 957144-77-3), diethofencarb, difenoconazole, dimethomorph, dipymetitrone, epoxiconazole, ethaboxam, fenarimol, fenhexamid, fluazinam, fludioxonil, fluindapyr, fluopyram, flusilazole, flutianil, flutriafol, fluxapyroxad, folpet, ipflufenoquin (Registry Number 1314008-27-9), iprodione, isofetamid, isoflucypram, isopyrazam, kresoxim-methyl, mancozeb, mandestrobin, meptyldinocap, metalaxyl (including metalaxyl-M/mefenoxam), mefentrifluconazole, metconazole, metrafenone, metyltetraprole (Registry Number 1472649-01-6), myclobutanil, oxathiapiprolin, penflufen, penthiopyrad, phosphorous acid (including salts thereof, e.g., fosetyl-aluminum), picoxystrobin, propiconazole, proquinazid, prothioconazole, pyridachlometyl (Registry Number 1358061-55-8), pyraclostrobin, pyrapropoyne (Registry Number 1803108-03-3), pyrimethanil, sedaxane spiroxamine, sulfur, tebuconazole, thiophanate-methyl, trifloxystrobin, zoxamide, α-(1-chlorocyclopropyl)-α-[2-(2,2-dichloro-cyclopropyl)ethyl]-1H-1,2,4-triazole-1-ethanol, N-[2-(2,4-dichlorophenyl)-2-methoxy-1-methylethyl]-3-(difluoromethyl)-1-methyl-1H-pyrazole-4-carboxamide, 3-(difluoromethyl)-N-(2,3-dihydro-1,1,3-trimethyl-1H-inden-4-yl)-1-methyl-1H-pyrazole-4-carboxamide, 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, 1,1-dimethylethyl N-[6-[[[[(1-methyl-1H-tetrazol-5-yl)phenylmethylene]amino]oxy]methyl]-2-pyridinyl]carbamate, 5-fluoro-2-[(4-fluorophenyl)methoxy]-4-pyrimidinamine, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chloro-phenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole (i.e. as Component (b) in compostions).

Generally Preferred for Better c

Generally preferred for better control of plant diseases caused by fungal plant pathogens (e.g., lower use rate or broader spectrum of plant pathogens controlled) or resistance management are mixtures of a compound of Formula 1, an N-oxide, or salt thereof, with a fungicidal compound selected from the group: amisulbrom, azoxystrobin, benzovindiflupyr, bixafen, boscalid, carbendazim, carboxin, chlorothalonil, copper hydroxide, cymoxanil, cyproconazole, difenoconazole, dimethomorph, dimoxystrobin, epoxiconazole, fenpropidin, fenpropimorph, florylpicoxamid, fluazinam, fludioxonil, flufenoxystrobin, fluindapyr, fluquinconazole, fluopicolide, fluoxastrobin, flutriafol, fluxapyroxad, ipconazole, ipfentrifluconazole, iprodione, kresoxim-methyl, mancozeb, metalaxyl, mefenoxam, mefentrifluconazole, metconazole, metominostrobin, myclobutanil, paclobutrazole, penflufen, picoxystrobin, prothioconazole, pydiflumetofen, pyraclostrobin, pyrametostrobin, pyraoxystrobin, pyriofenone, sedaxane, silthiofam, tebuconazole, thiabendazole, thiophanate-methyl, thiram, trifloxystrobin and triticonazole.

In the fungicidal compositions of the present invention, component (a) (i.e. at least one compound selected from compounds of Formula 1, N-oxides, and salts thereof) and component (b) are present in fungicidally effective amounts. The weight ratio of component (b) (i.e. one or more additional fungicidal compounds) to component (a) is generally between about 1:3000 to about 3000:1, and more typically between about 1:500 and about 500:1. Of note are compositions wherein the weight ratio of component (a) to component (b) is from about 125:1 to about 1:125. Of particular note are compositions wherein the weight ratio of component (a) to component (b) is from about 25:1 to about 1:25, or from about 5:1 to about 1:5. One skilled in the art can determine through simple experimentation the weight ratios and application rates of fungicidal compounds necessary for the desired spectrum of fungicidal protection and control. It will be evident that including additional fungicidal compounds in component (b) may expand the spectrum of plant diseases controlled beyond the spectrum controlled by component (a) alone. Furthermore, exemplify weight ratios for combinations of fungicidal compounds of the present invention are provided below in Tables A1-A15 and C1-C15. Table B1 below lists typical, more typical and most typical ranges of ratios involving particular fungicidal compounds of component (b).

Table A1 discloses specific mixtures of a Component (a) compound with a Component (b) compound. Component (a) compounds are identified by their compound number, see Index Tables A-B for a description of the compounds. The entries under the heading “Illustrative Ratios” disclose three specific weight ratios of Component (a) to Component (b) for the disclosed mixture. For example, the first line of Table A1 discloses a mixture of Compound 1 of the present invention with acibenzolar-S-methyl, with weight ratios of Compound 1 relative to acibenzolar-S-methyl of 1:1, 1:4 or 1:18.

| Component (a) | Component (b) | Illustrative Ratios(*) | | |
|---|---|---|---|---|
| Compound 1 | acibenzolar-S-methyl | 1:1 | 1:4 | 1:18 |
| Compound 1 | aldimorph | 7:1 | 3:1 | 1:1 |
| Compound 1 | ametoctradin | 3:1 | 1:1 | 1:3 |
| Compound 1 | amisulbrom | 1:1 | 1:2 | 1:6 |
| Compound 1 | anilazine | 22:1 | 8:1 | 4:1 |
| Compound 1 | azaconazole | 2:1 | 1:2 | 1:4 |
| Compound 1 | azoxystrobin | 3:1 | 1:1 | 1:3 |
| Compound 1 | benalaxyl | 1:1 | 1:2 | 1:6 |
| Compound 1 | benalaxyl-M | 1:1 | 1:3 | 1:8 |
| Compound 1 | benodanil | 4:1 | 2:1 | 1:2 |
| Compound 1 | benomyl | 11:1 | 4:1 | 1:1 |
| Compound 1 | benthiavalicarb | 1:1 | 1:4 | 1:12 |
| Compound 1 | benthiavalicarb-isopropyl | 1:1 | 1:4 | 1:12 |
| Compound 1 | bethoxazin | 15:1 | 5:1 | 2:1 |
| Compound 1 | binapacryl | 15:1 | 5:1 | 2:1 |
| Compound 1 | biphenyl | 15:1 | 5:1 | 2:1 |
| Compound 1 | bitertanol | 3:1 | 1:1 | 1:2 |
| Compound 1 | bixafen | 2:1 | 1:1 | 1:3 |
| Compound 1 | blasticidin-S | 1:4 | 1:12 | 1:30 |
| Compound 1 | Bordeaux mixture (tribasic copper sulfate) | 45:1 | 15:1 | 5:1 |
| Compound 1 | boscalid | 4:1 | 2:1 | 1:2 |
| Compound 1 | bromuconazole | 3:1 | 1:1 | 1:3 |
| Compound 1 | bupirimate | 1:3 | 1:10 | 1:30 |
| Compound 1 | captafol | 15:1 | 5:1 | 2:1 |
| Compound 1 | captan | 15:1 | 5:1 | 2:1 |
| Compound 1 | carbendazim | 11:1 | 4:1 | 2:1 |
| Compound 1 | carboxin | 4:1 | 2:1 | 1:2 |
| Compound 1 | carpropamid | 3:1 | 1:1 | 1:3 |
| Compound 1 | chloroneb | 100:1 | 35:1 | 14:1 |
| Compound 1 | chlorothalonil | 15:1 | 5:1 | 2:1 |
| Compound 1 | chlozolinate | 11:1 | 4:1 | 2:1 |
| Compound 1 | clotrimazole | 3:1 | 1:1 | 1:3 |
| Compound 1 | copper hydroxide | 45:1 | 15:1 | 5:1 |
| Compound 1 | copper oxychloride | 45:1 | 15:1 | 5:1 |
| Compound 1 | cyazofamid | 1:1 | 1:2 | 1:6 |
| Compound 1 | cyflufenamid | 1:2 | 1:6 | 1:24 |
| Compound 1 | cymoxanil | 1:1 | 1:2 | 1:5 |
| Compound 1 | cyproconazole | 1:1 | 1:2 | 1:6 |
| Compound 1 | cyprodinil | 4:1 | 2:1 | 1:2 |
| Compound 1 | dichlofluanid | 15:1 | 5:1 | 2:1 |
| Compound 1 | diclocymet | 15:1 | 5:1 | 2:1 |
| Compound 1 | diclomezine | 3:1 | 1:1 | 1:3 |
| Compound 1 | dicloran | 15:1 | 5:1 | 2:1 |
| Compound 1 | diethofencarb | 7:1 | 2:1 | 1:2 |
| Compound 1 | difenoconazole | 1:1 | 1:3 | 1:12 |
| Compound 1 | diflumetorim | 15:1 | 5:1 | 2:1 |
| Compound 1 | dimethirimol | 1:3 | 1:8 | 1:30 |
| Compound 1 | dimethomorph | 3:1 | 1:1 | 1:2 |
| Compound 1 | dimoxystrobin | 2:1 | 1:1 | 1:4 |
| Compound 1 | diniconazole | 1:1 | 1:3 | 1:8 |
| Compound 1 | diniconazole-M | 1:1 | 1:3 | 1:12 |
| Compound 1 | dinocap | 2:1 | 1:1 | 1:3 |
| Compound 1 | dithianon | 5:1 | 2:1 | 1:2 |
| Compound 1 | dodemorph | 7:1 | 3:1 | 1:1 |
| Compound 1 | dodine | 10:1 | 4:1 | 2:1 |
| Compound 1 | edifenphos | 3:1 | 1:1 | 1:3 |
| Compound 1 | enestroburin | 2:1 | 1:1 | 1:4 |
| Compound 1 | epoxiconazole | 1:1 | 1:3 | 1:7 |
| Compound 1 | etaconazole | 1:1 | 1:3 | 1:7 |
| Compound 1 | ethaboxam | 2:1 | 1:1 | 1:3 |
| Compound 1 | ethirimol | 7:1 | 3:1 | 1:1 |
| Compound 1 | etridiazole | 7:1 | 2:1 | 1:2 |
| Compound 1 | famoxadone | 2:1 | 1:1 | 1:4 |
| Compound 1 | fenamidone | 2:1 | 1:1 | 1:4 |
| Compound 1 | fenaminstrobin | 3:1 | 1:1 | 1:3 |
| Compound 1 | fenarimol | 1:2 | 1:7 | 1:24 |
| Compound 1 | fenbuconazole | 1:1 | 1:3 | 1:10 |
| Compound 1 | fenfuram | 4:1 | 1:1 | 1:2 |
| Compound 1 | fenhexamid | 10:1 | 4:1 | 2:1 |
| Compound 1 | fenoxanil | 15:1 | 4:1 | 1:1 |
| Compound 1 | fenpiclonil | 15:1 | 5:1 | 2:1 |
| Compound 1 | fenpropidin | 7:1 | 2:1 | 1:1 |
| Compound 1 | fenpropimorph | 7:1 | 2:1 | 1:1 |
| Compound 1 | fenpyrazamine | 3:1 | 1:1 | 1:3 |
| Compound 1 | fentin salt such as fentin acetate, | 3:1 | 1:1 | 1:3 |
| | fentin chloride or fentin hydroxide | | | |
| Compound 1 | ferbam | 30:1 | 10:1 | 4:1 |
| Compound 1 | ferimzone | 7:1 | 2:1 | 1:2 |
| Compound 1 | fluazinam | 3:1 | 1:1 | 1:2 |
| Compound 1 | fludioxonil | 2:1 | 1:1 | 1:4 |
| Compound 1 | flumetover | 3:1 | 1:1 | 1:2 |
| Compound 1 | flumorph | 3:1 | 1:1 | 1:3 |
| Compound 1 | fluopicolide | 1:1 | 1:2 | 1:6 |
| Compound 1 | fluopyram | 3:1 | 1:1 | 1:3 |
| Compound 1 | fluoroimide | 37:1 | 14:1 | 5:1 |
| Compound 1 | fluoxastrobin | 1:1 | 1:2 | 1:6 |
| Compound 1 | fluquinconazole | 1:1 | 1:2 | 1:4 |
| Compound 1 | flusilazole | 3:1 | 1:1 | 1:3 |
| Compound 1 | flusulfamide | 15:1 | 5:1 | 2:1 |
| Compound 1 | flutianil | 1:1 | 1:2 | 1:6 |
| Compound 1 | flutolanil | 4:1 | 1:1 | 1:2 |
| Compound 1 | flutriafol | 1:1 | 1:2 | 1:4 |
| Compound 1 | fluxapyroxad | 2:1 | 1:1 | 1:3 |
| Compound 1 | folpet | 15:1 | 5:1 | 2:1 |
| Compound 1 | fosetyl-aluminum | 30:1 | 12:1 | 5:1 |
| Compound 1 | fuberidazole | 11:1 | 4:1 | 2:1 |
| Compound 1 | furalaxyl | 1:1 | 1:2 | 1:6 |
| Compound 1 | furametpyr | 15:1 | 5:1 | 2:1 |
| Compound 1 | guazatine | 15:1 | 5:1 | 2:1 |
| Compound 1 | hexaconazole | 1:1 | 1:2 | 1:5 |
| Compound 1 | hymexazol | 75:1 | 25:1 | 9:1 |
| Compound 1 | imazalil | 1:1 | 1:2 | 1:5 |
| Compound 1 | imibenconazole | 1:1 | 1:2 | 1:5 |
| Compound 1 | iminoctadine | 15:1 | 4:1 | 1:1 |
| Compound 1 | iodocarb | 15:1 | 5:1 | 2:1 |
| Compound 1 | ipconazole | 1:1 | 1:2 | 1:5 |
| Compound 1 | iprobenfos | 15:1 | 5:1 | 2:1 |
| Compound 1 | iprodione | 15:1 | 5:1 | 2:1 |
| Compound 1 | iprovalicarb | 2:1 | 1:1 | 1:3 |
| Compound 1 | isoprothiolane | 45:1 | 15:1 | 5:1 |
| Compound 1 | isopyrazam | 2:1 | 1:1 | 1:3 |
| Compound 1 | isotianil | 2:1 | 1:1 | 1:3 |
| Compound 1 | kasugamycin | 1:2 | 1:7 | 1:24 |
| Compound 1 | kresoxim-methyl | 2:1 | 1:1 | 1:4 |
| Compound 1 | mancozeb | 22:1 | 7:1 | 3:1 |
| Compound 1 | mandipropamid | 2:1 | 1:1 | 1:4 |
| Compound 1 | maneb | 22:1 | 7:1 | 3:1 |
| Compound 1 | mepanipyrim | 6:1 | 2:1 | 1:1 |
| Compound 1 | mepronil | 1:1 | 1:2 | 1:6 |
| Compound 1 | meptyldinocap | 2:1 | 1:1 | 1:3 |
| Compound 1 | metalaxyl | 1:1 | 1:2 | 1:6 |
| Compound 1 | metalaxyl-M | 1:1 | 1:4 | 1:12 |
| Compound 1 | metconazole | 1:1 | 1:2 | 1:6 |
| Compound 1 | methasulfocarb | 15:1 | 5:1 | 2:1 |
| Compound 1 | metiram | 15:1 | 5:1 | 2:1 |
| Compound 1 | metominostrobin | 3:1 | 1:1 | 1:3 |
| Compound 1 | metrafenone | 2:1 | 1:1 | 1:4 |
| Compound 1 | myclobutanil | 1:1 | 1:3 | 1:8 |
| Compound 1 | naftifine | 15:1 | 5:1 | 2:1 |
| Compound 1 | neo-asozin (ferric methanearsonate) | 15:1 | 5:1 | 2:1 |
| Compound 1 | nuarimol | 3:1 | 1:1 | 1:3 |
| Compound 1 | octhilinone | 15:1 | 4:1 | 1:1 |
| Compound 1 | ofurace | 1:1 | 1:2 | 1:6 |
| Compound 1 | orysastrobin | 3:1 | 1:1 | 1:3 |
| Compound 1 | oxadixyl | 1:1 | 1:2 | 1:6 |
| Compound 1 | oxolinic acid | 7:1 | 2:1 | 1:2 |
| Compound 1 | oxpoconazole | 1:1 | 1:2 | 1:5 |
| Compound 1 | oxycarboxin | 4:1 | 1:1 | 1:2 |
| Compound 1 | oxytetracycline | 3:1 | 1:1 | 1:3 |
| Compound 1 | pefurazoate | 15:1 | 5:1 | 2:1 |
| Compound 1 | penconazole | 1:2 | 1:6 | 1:15 |
| Compound 1 | pencycuron | 11:1 | 4:1 | 2:1 |
| Compound 1 | penflufen | 2:1 | 1:1 | 1:3 |
| Compound 1 | penthiopyrad | 2:1 | 1:1 | 1:3 |
| Compound 1 | phosphorous acid or a salt thereof | 15:1 | 6:1 | 2:1 |
| Compound 1 | phthalide | 15:1 | 6:1 | 2:1 |
| Compound 1 | picoxystrobin | 1:1 | 1:2 | 1:5 |
| Compound 1 | piperalin | 3:1 | 1:1 | 1:3 |
| Compound 1 | polyoxin | 3:1 | 1:1 | 1:3 |
| Compound 1 | probenazole | 3:1 | 1:1 | 1:3 |
| Compound 1 | prochloraz | 7:1 | 2:1 | 1:2 |
| Compound 1 | procymidone | 11:1 | 4:1 | 2:1 |
| Compound 1 | propamocarb or propamocarb-hydrochloride | 10:1 | 4:1 | 2:1 |
| Compound 1 | propiconazole | 1:1 | 1:2 | 1:5 |
| Compound 1 | propineb | 11:1 | 4:1 | 2:1 |
| Compound 1 | proquinazid | 1:1 | 1:3 | 1:12 |
| Compound 1 | prothiocarb | 3:1 | 1:1 | 1:3 |
| Compound 1 | prothioconazole | 1:1 | 1:2 | 1:5 |
| Compound 1 | pyraclostrobin | 2:1 | 1:1 | 1:4 |
| Compound 1 | pyrametostrobin | 2:1 | 1:1 | 1:4 |
| Compound 1 | pyraoxystrobin | 2:1 | 1:1 | 1:4 |
| Compound 1 | pyrazophos | 15:1 | 4:1 | 1:1 |
| Compound 1 | pyribencarb | 4:1 | 1:1 | 1:2 |
| Compound 1 | pyributicarb | 15:1 | 4:1 | 1:1 |
| Compound 1 | pyrifenox | 3:1 | 1:1 | 1:3 |
| Compound 1 | pyrimethanil | 3:1 | 1:1 | 1:2 |
| Compound 1 | pyriofenone | 2:1 | 1:1 | 1:4 |
| Compound 1 | pyrisoxazole | 3:1 | 1:1 | 1:3 |
| Compound 1 | pyroquilon | 3:1 | 1:1 | 1:3 |
| Compound 1 | pyrrolnitrin | 15:1 | 5:1 | 2:1 |
| Compound 1 | quinconazole | 1:1 | 1:2 | 1:4 |
| Compound 1 | quinomethionate | 15:1 | 5:1 | 2:1 |
| Compound 1 | quinoxyfen | 1:1 | 1:2 | 1:6 |
| Compound 1 | quintozene | 15:1 | 5:1 | 2:1 |
| Compound 1 | silthiofam | 2:1 | 1:1 | 1:4 |
| Compound 1 | simeconazole | 1:1 | 1:2 | 1:5 |
| Compound 1 | spiroxamine | 5:1 | 2:1 | 1:2 |
| Compound 1 | streptomycin | 3:1 | 1:1 | 1:3 |
| Compound 1 | sulfur | 75:1 | 25:1 | 9:1 |
| Compound 1 | tebuconazole | 1:1 | 1:2 | 1:5 |
| Compound 1 | tebufloquin | 3:1 | 1:1 | 1:3 |
| Compound 1 | tecloftalam | 15:1 | 5:1 | 2:1 |
| Compound 1 | tecnazene | 15:1 | 5:1 | 2:1 |
| Compound 1 | terbinafine | 15:1 | 5:1 | 2:1 |
| Compound 1 | tetraconazole | 1:1 | 1:2 | 1:5 |
| Compound 1 | thiabendazole | 11:1 | 4:1 | 2:1 |
| Compound 1 | thifluzamide | 3:1 | 1:1 | 1:3 |
| Compound 1 | thiophanate | 11:1 | 4:1 | 2:1 |
| Compound 1 | thiophanate-methyl | 11:1 | 4:1 | 2:1 |
| Compound 1 | thiram | 37:1 | 14:1 | 5:1 |
| Compound 1 | tiadinil | 2:1 | 1:1 | 1:3 |
| Compound 1 | tolclofos-methyl | 37:1 | 14:1 | 5:1 |
| Compound 1 | tolnifanide | 3:1 | 1:1 | 1:3 |
| Compound 1 | tolylfluanid | 15:1 | 5:1 | 2:1 |
| Compound 1 | triadimefon | 1:1 | 1:2 | 1:5 |
| Compound 1 | triadimenol | 1:1 | 1:2 | 1:5 |
| Compound 1 | triarimol | 1:2 | 1:7 | 1:24 |
| Compound 1 | triazoxide | 15:1 | 5:1 | 2:1 |
| Compound 1 | tricyclazole | 3:1 | 1:1 | 1:3 |
| Compound 1 | tridemorph | 7:1 | 2:1 | 1:1 |
| Compound 1 | trifloxystrobin | 2:1 | 1:1 | 1:4 |
| Compound 1 | triflumizole | 3:1 | 1:1 | 1:3 |
| Compound 1 | triforine | 3:1 | 1:1 | 1:3 |
| Compound 1 | trimorphamide | 7:1 | 2:1 | 1:2 |
| Compound 1 | triticonazole | 1:1 | 1:2 | 1:5 |
| Compound 1 | uniconazole | 1:1 | 1:2 | 1:5 |
| Compound 1 | validamycin | 3:1 | 1:1 | 1:3 |
| Compound 1 | valifenalate | 2:1 | 1:1 | 1:4 |
| Compound 1 | vinclozolin | 15:1 | 6:1 | 2:1 |
| Compound 1 | zineb | 37:1 | 14:1 | 5:1 |
| Compound 1 | ziram | 37:1 | 14:1 | 5:1 |
| Compound 1 | zoxamide | 2:1 | 1:1 | 1:4 |
| Compound 1 | 5-chloro-6-(2,4,6-trifluorophenyl)-7-(4-methylpiperidin- | 1:1 | 1:2 | 1:6 |
| | 1-yl)[1,2,4]triazolo[1,5-a]pyrimidine (DPX-BAS600F) | | | |
| Compound 1 | N-[2-[4-[[3-(4-chlorophenyl)-2-propyn-1-yl]oxy]-3-methoxy- | 2:1 | 1:1 | 1:4 |
| | phenyl]ethyl]-3-methyl-2-[(methylsulfonyl)amino]- | | | |
| | butanamide | | | |
| Compound 1 | N-[2-[4-[[3-(4-chlorophenyl)-2-propyn-1-yl]oxy]-3-methoxy- | 2:1 | 1:1 | 1:4 |
| | phenyl]ethyl]-3-methyl-2-[(ethylsulfonyl)amino]butanamide | | | |
| Compound 1 | 4-fluorophenyl N-[1-[[[1-(4-cyanophenyl)ethyl]sulfonyl]- | 2:1 | 1:1 | 1:4 |
| | methyl]propyl]carbamate | | | |
| Compound 1 | α-[methoxyimino]-N-methyl-2-[[[1-[3-(trifluoromethyl)- | 3:1 | 1:1 | 1:3 |
| | phenyl]ethoxy]imino]methyl]benzeneacetamide | | | |
| Compound 1 | N′-[4-[4-chloro-3-(trifluoromethyl)phenoxy]-2,5-dimethyl- | 3:1 | 1:1 | 1:3 |
| | phenyl]-N-ethyl-N-methylmethanimidamide | | | |

(*)Ratios of Component (b) relative to Component (a) by weight.

Tables A2 through A15 are each constructed the same as Table A1 above except that entries below the “Component (a)” column heading are replaced with the respective Component (a) Column Entry shown below. Thus, for example, in Table A2 the entries below the “Component (a)” column heading all recite “Compound 18”. Therefore, the first entry in Table A2 specifically discloses a mixture of Compound 18 with acibenzolar-S-methyl. Tables A3 through A15 are constructed similarly.

| Table Number | Component (a) Column Entry |
|---|---|
| A2 | Compound 18 |
| A3 | Compound 19 |
| A4 | Compound 23 |
| A5 | Compound 57 |
| A6 | Compound 60 |
| A7 | Compound 68 |
| A8 | Compound 72 |
| A9 | Compound 73 |
| A10 | Compound 93 |
| A11 | Compound 111 |
| A12 | Compound 112 |
| A13 | Compound 118 |
| A14 | Compound 121 |
| A15 | Compound 127 |

Table B1 lists combinations of a Component (b) compound with Component (a) compound illustrative of the mixtures, compositions and methods of the present invention. The first column of Table B1 lists the specific Component (b) compound (e.g., “acibenzolar-S-methyl” is the first entry). The second, third and fourth columns of Table B1 lists ranges of weight ratios for rates at which the Component (a) compound is typically applied to a field-grown crop relative to Component (b). Thus, for example, the first line of Table B1 discloses the combination of a compound of Component (a) with acibenzolar-S-methyl is typically applied in a weight ratio of Component (a) to Component (b) of between 2:1 to 1:180, more typically between 1:1 to 1:60, and most typically between 1:1 to 1:18. The remaining lines of Table B1 are to be construed similarly. Of particular note is a composition comprising a mixture of any one of the compounds listed in Embodiment 97 as Component (a) with a compound listed in the Component (b) column of Table B1 according to the weight ratios disclosed in Table B1. Table B1 thus supplements the specific ratios disclosed in Tables A1 through A15 with ranges of ratios for these combinations.

TABLE B1

| | Typical | More Typical | Most Typical |
|---|---|---|---|
| Component (b) | Weight Ratio | Weight Ratio | Weight Ratio |
| acibenzolar-S-methyl | 2:1 to 1:180 | 1:1 to 1:60 | 1:1 to 1:18 |
| aldimorph | 30:1 to 1:3 | 10:1 to 1:1 | 7:1 to 1:1 |
| ametoctradin | 9:1 to 1:18 | 3:1 to 1:6 | 3:1 to 1:3 |
| amisulbrom | 6:1 to 1:18 | 2:1 to 1:6 | 1:1 to 1:6 |
| anilazine | 90:1 to 2:1 | 30:1 to 4:1 | 22:1 to 4:1 |
| azaconazole | 7:1 to 1:18 | 2:1 to 1:6 | 2:1 to 1:4 |
| azoxystrobin | 9:1 to 1:12 | 3:1 to 1:4 | 3:1 to 1:3 |
| benalaxyl | 4:1 to 1:18 | 1:1 to 1:6 | 1:1 to 1:6 |
| benalaxyl-M | 4:1 to 1:36 | 1:1 to 1:12 | 1:1 to 1:8 |
| benodanil | 18:1 to 1:6 | 6:1 to 1:2 | 4:1 to 1:2 |
| benomyl | 45:1 to 1:4 | 15:1 to 1:1 | 11:1 to 1:1 |
| benthiavalicarb or benthiavalicarb- | 2:1 to 1:36 | 1:1 to 1:12 | 1:1 to 1:12 |
| isopropyl | | | |
| bethoxazin | 150:1 to 1:36 | 50:1 to 1:12 | 15:1 to 2:1 |
| binapacryl | 150:1 to 1:36 | 50:1 to 1:12 | 15:1 to 2:1 |
| biphenyl | 150:1 to 1:36 | 50:1 to 1:12 | 15:1 to 2:1 |
| bitertanol | 15:1 to 1:5 | 5:1 to 1:2 | 3:1 to 1:2 |
| bixafen | 12:1 to 1:9 | 4:1 to 1:3 | 2:1 to 1:3 |
| blasticidin-S | 3:1 to 1:90 | 1:1 to 1:30 | 1:4 to 1:30 |
| boscalid | 18:1 to 1:6 | 6:1 to 1:2 | 4:1 to 1:2 |
| bromuconazole | 15:1 to 1:9 | 5:1 to 1:3 | 3:1 to 1:3 |
| bupirimate | 3:1 to 1:90 | 1:1 to 1:30 | 1:3 to 1:30 |
| captafol | 90:1 to 1:4 | 30:1 to 1:2 | 15:1 to 2:1 |
| captan | 90:1 to 1:4 | 30:1 to 1:2 | 15:1 to 2:1 |
| carbendazim | 45:1 to 1:4 | 15:1 to 1:2 | 11:1 to 2:1 |
| carboxin | 18:1 to 1:6 | 6:1 to 1:2 | 4:1 to 1:2 |
| carpropamid | 15:1 to 1:9 | 5:1 to 1:3 | 3:1 to 1:3 |
| chloroneb | 300:1 to 2:1 | 100:1 to 4:1 | 100:1 to 14:1 |
| chlorothalonil | 90:1 to 1:4 | 30:1 to 1:2 | 15:1 to 2:1 |
| chlozolinate | 45:1 to 1:2 | 15:1 to 2:1 | 11:1 to 2:1 |
| clotrimazole | 15:1 to 1:9 | 5:1 to 1:3 | 3:1 to 1:3 |
| copper salts such as Bordeaux mixture | 450:1 to 1:1 | 150:1 to 4:1 | 45:1 to 5:1 |
| (tribasic copper sulfate), copper | | | |
| oxychloride, copper sulfate and copper | | | |
| hydroxide | | | |
| cyazofamid | 4:1 to 1:18 | 1:1 to 1:6 | 1:1 to 1:6 |
| cyflufenamid | 1:1 to 1:90 | 1:2 to 1:30 | 1:2 to 1:24 |
| cymoxanil | 6:1 to 1:18 | 2:1 to 1:6 | 1:1 to 1:5 |
| cyproconazole | 4:1 to 1:18 | 1:1 to 1:6 | 1:1 to 1:6 |
| cyprodinil | 22:1 to 1:9 | 7:1 to 1:3 | 4:1 to 1:2 |
| dichlofluanid | 150:1 to 1:36 | 50:1 to 1:12 | 15:1 to 2:1 |
| diclocymet | 150:1 to 1:36 | 50:1 to 1:12 | 15:1 to 2:1 |
| diclomezine | 15:1 to 1:9 | 5:1 to 1:3 | 3:1 to 1:3 |
| dicloran | 150:1 to 1:36 | 50:1 to 1:12 | 15:1 to 2:1 |
| diethofencarb | 22:1 to 1:9 | 7:1 to 1:3 | 7:1 to 1:2 |
| difenoconazole | 4:1 to 1:36 | 1:1 to 1:12 | 1:1 to 1:12 |
| diflumetorim | 150:1 to 1:36 | 50:1 to 1:12 | 15:1 to 2:1 |
| dimethirimol | 3:1 to 1:90 | 1:1 to 1:30 | 1:3 to 1:30 |
| dimethomorph | 9:1 to 1:6 | 3:1 to 1:2 | 3:1 to 1:2 |
| dimoxystrobin | 9:1 to 1:18 | 3:1 to 1:6 | 2:1 to 1:4 |
| diniconazole | 3:1 to 1:36 | 1:1 to 1:12 | 1:1 to 1:8 |
| diniconazole M | 3:1 to 1:90 | 1:1 to 1:30 | 1:1 to 1:12 |
| dinocap | 7:1 to 1:9 | 2:1 to 1:3 | 2:1 to 1:3 |
| dithianon | 15:1 to 1:4 | 5:1 to 1:2 | 5:1 to 1:2 |
| dodemorph | 30:1 to 1:3 | 10:1 to 1:1 | 7:1 to 1:1 |
| dodine | 30:1 to 1:2 | 10:1 to 2:1 | 10:1 to 2:1 |
| edifenphos | 30:1 to 1:9 | 10:1 to 1:3 | 3:1 to 1:3 |
| enestroburin | 9:1 to 1:18 | 3:1 to 1:6 | 2:1 to 1:4 |
| epoxiconazole | 3:1 to 1:36 | 1:1 to 1:12 | 1:1 to 1:7 |
| etaconazole | 3:1 to 1:36 | 1:1 to 1:12 | 1:1 to 1:7 |
| ethaboxam | 7:1 to 1:9 | 2:1 to 1:3 | 2:1 to 1:3 |
| ethirimol | 30:1 to 1:3 | 10:1 to 1:1 | 7:1 to 1:1 |
| etridiazole | 30:1 to 1:9 | 10:1 to 1:3 | 7:1 to 1:2 |
| famoxadone | 9:1 to 1:18 | 3:1 to 1:6 | 2:1 to 1:4 |
| fenamidone | 6:1 to 1:18 | 2:1 to 1:6 | 2:1 to 1:4 |
| fenaminstrobin | 9:1 to 1:18 | 3:1 to 1:6 | 3:1 to 1:3 |
| fenarimol | 3:1 to 1:90 | 1:1 to 1:30 | 1:2 to 1:24 |
| fenbuconazole | 3:1 to 1:30 | 1:1 to 1:10 | 1:1 to 1:10 |
| fenfuram | 18:1 to 1:6 | 6:1 to 1:2 | 4:1 to 1:2 |
| fenhexamid | 30:1 to 1:2 | 10:1 to 2:1 | 10:1 to 2:1 |
| fenoxanil | 150:1 to 1:36 | 50:1 to 1:12 | 15:1 to 1:1 |
| fenpiclonil | 75:1 to 1:9 | 25:1 to 1:3 | 15:1 to 2:1 |
| fenpropidin | 30:1 to 1:3 | 10:1 to 1:1 | 7:1 to 1:1 |
| fenpropimorph | 30:1 to 1:3 | 10:1 to 1:1 | 7:1 to 1:1 |
| fenpyrazamine | 100:1 to 1:100 | 10:1 to 1:10 | 3:1 to 1:3 |
| fentin salt such as the acetate, chloride or | 15:1 to 1:9 | 5:1 to 1:3 | 3:1 to 1:3 |
| hydroxide | | | |
| ferbam | 300:1 to 1:2 | 100:1 to 2:1 | 30:1 to 4:1 |
| ferimzone | 30:1 to 1:5 | 10:1 to 1:2 | 7:1 to 1:2 |
| fluazinam | 22:1 to 1:5 | 7:1 to 1:2 | 3:1 to 1:2 |
| fludioxonil | 7:1 to 1:12 | 2:1 to 1:4 | 2:1 to 1:4 |
| flumetover | 9:1 to 1:6 | 3:1 to 1:2 | 3:1 to 1:2 |
| flumorph | 9:1 to 1:18 | 3:1 to 1:6 | 3:1 to 1:3 |
| fluopicolide | 3:1 to 1:18 | 1:1 to 1:6 | 1:1 to 1:6 |
| fluopyram | 15:1 to 1:90 | 5:1 to 1:30 | 3:1 to 1:3 |
| fluoromide | 150:1 to 2:1 | 50:1 to 4:1 | 37:1 to 5:1 |
| fluoxastrobin | 4:1 to 1:18 | 1:1 to 1:6 | 1:1 to 1:6 |
| fluquinconazole | 4:1 to 1:12 | 1:1 to 1:4 | 1:1 to 1:4 |
| flusilazole | 15:1 to 1:9 | 5:1 to 1:3 | 3:1 to 1:3 |
| flusulfamide | 90:1 to 1:2 | 30:1 to 2:1 | 15:1 to 2:1 |
| flutianil | 7:1 to 1:36 | 2:1 to 1:12 | 1:1 to 1:6 |
| flutolanil | 18:1 to 1:6 | 6:1 to 1:2 | 4:1 to 1:2 |
| flutriafol | 4:1 to 1:12 | 1:1 to 1:4 | 1:1 to 1:4 |
| fluxapyroxad | 12:1 to 1:9 | 4:1 to 1:3 | 2:1 to 1:3 |
| folpet | 90:1 to 1:4 | 30:1 to 1:2 | 15:1 to 2:1 |
| fosetyl-aluminum | 225:1 to 2:1 | 75:1 to 5:1 | 30:1 to 5:1 |
| fuberidazole | 45:1 to 1:4 | 15:1 to 1:2 | 11:1 to 2:1 |
| furalaxyl | 15:1 to 1:45 | 5:1 to 1:15 | 1:1 to 1:6 |
| furametpyr | 150:1 to 1:36 | 50:1 to 1:12 | 15:1 to 2:1 |
| guazatine or iminoctadine | 150:1 to 1:36 | 50:1 to 1:12 | 15:1 to 2:1 |
| hexaconazole | 15:1 to 1:36 | 5:1 to 1:12 | 1:1 to 1:5 |
| hymexazol | 225:1 to 2:1 | 75:1 to 4:1 | 75:1 to 9:1 |
| imazalil | 7:1 to 1:18 | 2:1 to 1:6 | 1:1 to 1:5 |
| imibenconazole | 15:1 to 1:36 | 5:1 to 1:12 | 1:1 to 1:5 |
| iodocarb | 150:1 to 1:36 | 50:1 to 1:12 | 15:1 to 2:1 |
| ipconazole | 15:1 to 1:36 | 5:1 to 1:12 | 1:1 to 1:5 |
| iprobenfos | 150:1 to 1:36 | 50:1 to 1:12 | 15:1 to 2:1 |
| iprodione | 120:1 to 1:2 | 40:1 to 2:1 | 15:1 to 2:1 |
| iprovalicarb | 9:1 to 1:9 | 3:1 to 1:3 | 2:1 to 1:3 |
| isoprothiolane | 150:1 to 2:1 | 50:1 to 4:1 | 45:1 to 5:1 |
| isopyrazam | 12:1 to 1:9 | 4:1 to 1:3 | 2:1 to 1:3 |
| isotianil | 12:1 to 1:9 | 4:1 to 1:3 | 2:1 to 1:3 |
| kasugamycin | 7:1 to 1:90 | 2:1 to 1:30 | 1:2 to 1:24 |
| kresoxim-methyl | 7:1 to 1:18 | 2:1 to 1:6 | 2:1 to 1:4 |
| mancozeb | 180:1 to 1:3 | 60:1 to 2:1 | 22:1 to 3:1 |
| mandipropamid | 6:1 to 1:18 | 2:1 to 1:6 | 2:1 to 1:4 |
| maneb | 180:1 to 1:3 | 60:1 to 2:1 | 22:1 to 3:1 |
| mepanipyrim | 18:1 to 1:3 | 6:1 to 1:1 | 6:1 to 1:1 |
| mepronil | 7:1 to 1:36 | 2:1 to 1:12 | 1:1 to 1:6 |
| meptyldinocap | 7:1 to 1:9 | 2:1 to 1:3 | 2:1 to 1:3 |
| metalaxyl | 15:1 to 1:45 | 5:1 to 1:15 | 1:1 to 1:6 |
| metalaxyl-M | 7:1 to 1:90 | 2:1 to 1:30 | 1:1 to 1:12 |
| metconazole | 3:1 to 1:18 | 1:1 to 1:6 | 1:1 to 1:6 |
| methasulfocarb | 150:1 to 1:36 | 50:1 to 1:12 | 15:1 to 1:1 |
| metiram | 150:1 to 1:36 | 50:1 to 1:12 | 15:1 to 1:1 |
| metominostrobin | 9:1 to 1:12 | 3:1 to 1:4 | 3:1 to 1:3 |
| metrafenone | 6:1 to 1:12 | 2:1 to 1:4 | 2:1 to 1:4 |
| myclobutanil | 5:1 to 1:26 | 1:1 to 1:9 | 1:1 to 1:8 |
| naftifine | 150:1 to 1:36 | 50:1 to 1:12 | 15:1 to 2:1 |
| neo-asozin (ferric methanearsonate) | 150:1 to 1:36 | 50:1 to 1:12 | 15:1 to 2:1 |
| nuarimol | 15:1 to 1:9 | 5:1 to 1:3 | 3:1 to 1:3 |
| octhilinone | 150:1 to 1:36 | 50:1 to 1:12 | 15:1 to 1:1 |
| ofurace | 15:1 to 1:45 | 5:1 to 1:15 | 1:1 to 1:6 |
| orysastrobin | 9:1 to 1:12 | 3:1 to 1:4 | 3:1 to 1:3 |
| oxadixyl | 15:1 to 1:45 | 5:1 to 1:15 | 1:1 to 1:6 |
| oxolinic acid | 30:1 to 1:9 | 10:1 to 1:3 | 7:1 to 1:2 |
| oxpoconazole | 15:1 to 1:36 | 5:1 to 1:12 | 1:1 to 1:5 |
| oxycarboxin | 18:1 to 1:6 | 6:1 to 1:2 | 4:1 to 1:2 |
| oxytetracycline | 15:1 to 1:9 | 5:1 to 1:3 | 3:1 to 1:3 |
| pefurazoate | 150:1 to 1:36 | 50:1 to 1:12 | 15:1 to 2:1 |
| penconazole | 1:1 to 1:45 | 1:2 to 1:15 | 1:2 to 1:15 |
| pencycuron | 150:1 to 1:2 | 50:1 to 2:1 | 11:1 to 2:1 |
| penflufen | 12:1 to 1:9 | 4:1 to 1:3 | 2:1 to 1:3 |
| penthiopyrad | 12:1 to 1:9 | 4:1 to 1:3 | 2:1 to 1:3 |
| phosphorous acid and salts thereof | 150:1 to 1:36 | 50:1 to 1:12 | 15:1 to 2:1 |
| phthalide | 150:1 to 1:36 | 50:1 to 1:12 | 15:1 to 2:1 |
| picoxystrobin | 7:1 to 1:18 | 2:1 to 1:6 | 1:1 to 1:5 |
| piperalin | 15:1 to 1:9 | 5:1 to 1:3 | 3:1 to 1:3 |
| polyoxin | 15:1 to 1:9 | 5:1 to 1:3 | 3:1 to 1:3 |
| probenazole | 15:1 to 1:9 | 5:1 to 1:3 | 3:1 to 1:3 |
| prochloraz | 22:1 to 1:4 | 7:1 to 1:1 | 7:1 to 1:2 |
| procymidone | 45:1 to 1:3 | 15:1 to 1:1 | 11:1 to 2:1 |
| propamocarb or propamocarb- | 30:1 to 1:2 | 10:1 to 2:1 | 10:1 to 2:1 |
| hydrochloride | | | |
| propiconazole | 4:1 to 1:18 | 1:1 to 1:6 | 1:1 to 1:5 |
| propineb | 45:1 to 1:2 | 15:1 to 2:1 | 11:1 to 2:1 |
| proquinazid | 3:1 to 1:36 | 1:1 to 1:12 | 1:1 to 1:12 |
| prothiocarb | 9:1 to 1:18 | 3:1 to 1:6 | 3:1 to 1:3 |
| prothioconazole | 6:1 to 1:18 | 2:1 to 1:6 | 1:1 to 1:5 |
| pyraclostrobin | 9:1 to 1:18 | 3:1 to 1:6 | 2:1 to 1:4 |
| pyrametostrobin | 9:1 to 1:18 | 3:1 to 1:6 | 2:1 to 1:4 |
| pyraoxystrobin | 9:1 to 1:18 | 3:1 to 1:6 | 2:1 to 1:4 |
| pyrazophos | 150:1 to 1:36 | 50:1 to 1:12 | 15:1 to 1:1 |
| pyribencarb | 15:1 to 1:6 | 5:1 to 1:2 | 4:1 to 1:2 |
| pyrifenox | 15:1 to 1:9 | 5:1 to 1:3 | 3:1 to 1:3 |
| pyrimethanil | 30:1 to 1:6 | 10:1 to 1:2 | 3:1 to 1:2 |
| pyriofenone | 6:1 to 1:12 | 2:1 to 1:4 | 2:1 to 1:4 |
| pyrisoxazole | 15:1 to 1:9 | 5:1 to 1:3 | 3:1 to 1:3 |
| pyroquilon | 15:1 to 1:9 | 5:1 to 1:3 | 3:1 to 1:3 |
| pyrrolnitrin | 150:1 to 1:36 | 50:1 to 1:12 | 15:1 to 2:1 |
| quinconazole | 4:1 to 1:12 | 1:1 to 1:4 | 1:1 to 1:4 |
| quinmethionate | 150:1 to 1:36 | 50:1 to 1:12 | 15:1 to 2:1 |
| quinoxyfen | 4:1 to 1:18 | 1:1 to 1:6 | 1:1 to 1:6 |
| quintozene | 150:1 to 1:36 | 50:1 to 1:12 | 15:1 to 2:1 |
| silthiofam | 7:1 to 1:18 | 2:1 to 1:6 | 2:1 to 1:4 |
| simeconazole | 15:1 to 1:36 | 5:1 to 1:12 | 1:1 to 1:5 |
| spiroxamine | 22:1 to 1:4 | 7:1 to 1:2 | 5:1 to 1:2 |
| streptomycin | 15:1 to 1:9 | 5:1 to 1:3 | 3:1 to 1:3 |
| sulfur | 300:1 to 3:1 | 100:1 to 9:1 | 75:1 to 9:1 |
| tebuconazole | 7:1 to 1:18 | 2:1 to 1:6 | 1:1 to 1:5 |
| tebufloquin | 100:1 to 1:100 | 10:1 to 1:10 | 3:1 to 1:3 |
| tecloftalam | 150:1 to 1:36 | 50:1 to 1:12 | 15:1 to 2:1 |
| tecnazene | 150:1 to 1:36 | 50:1 to 1:12 | 15:1 to 2:1 |
| terbinafine | 150:1 to 1:36 | 50:1 to 1:12 | 15:1 to 2:1 |
| tetraconazole | 15:1 to 1:36 | 5:1 to 1:12 | 1:1 to 1:5 |
| thiabendazole | 45:1 to 1:4 | 15:1 to 1:2 | 11:1 to 2:1 |
| thifluzamide | 15:1 to 1:9 | 5:1 to 1:3 | 3:1 to 1:3 |
| thiophanate | 45:1 to 1:3 | 15:1 to 2:1 | 11:1 to 2:1 |
| thiophanate-methyl | 45:1 to 1:3 | 15:1 to 2:1 | 11:1 to 2:1 |
| thiram | 150:1 to 1:2 | 50:1 to 2:1 | 37:1 to 5:1 |
| tiadinil | 12:1 to 1:9 | 4:1 to 1:3 | 2:1 to 1:3 |
| tolclofos-methyl | 150:1 to 1:2 | 50:1 to 2:1 | 37:1 to 5:1 |
| tolnifanide | 15:1 to 1:18 | 5:1 to 1:6 | 3:1 to 1:3 |
| tolylfluanid | 150:1 to 1:36 | 50:1 to 1:12 | 15:1 to 2:1 |
| triadimefon | 15:1 to 1:36 | 5:1 to 1:12 | 1:1 to 1:5 |
| triadimenol | 15:1 to 1:36 | 5:1 to 1:12 | 1:1 to 1:5 |
| triarimol | 3:1 to 1:90 | 1:1 to 1:30 | 1:2 to 1:24 |
| triazoxide | 150:1 to 1:36 | 50:1 to 1:12 | 15:1 to 2:1 |
| tricyclazole | 15:1 to 1:9 | 5:1 to 1:3 | 3:1 to 1:3 |
| tridemorph | 30:1 to 1:3 | 10:1 to 1:1 | 7:1 to 1:1 |
| trifloxystrobin | 6:1 to 1:18 | 2:1 to 1:6 | 2:1 to 1:4 |
| triflumizole | 15:1 to 1:9 | 5:1 to 1:3 | 3:1 to 1:3 |
| triforine | 15:1 to 1:9 | 5:1 to 1:3 | 3:1 to 1:3 |
| trimorphamide | 45:1 to 1:9 | 15:1 to 1:3 | 7:1 to 1:2 |
| triticonazole | 15:1 to 1:36 | 5:1 to 1:12 | 1:1 to 1:5 |
| uniconazole | 15:1 to 1:36 | 5:1 to 1:12 | 1:1 to 1:5 |
| validamycin | 150:1 to 1:36 | 50:1 to 1:12 | 3:1 to 1:3 |
| valifenalate | 6:1 to 1:18 | 2:1 to 1:6 | 2:1 to 1:4 |
| vinclozolin | 120:1 to 1:2 | 40:1 to 2:1 | 15:1 to 2:1 |
| zineb | 150:1 to 1:2 | 50:1 to 2:1 | 37:1 to 5:1 |
| ziram | 150:1 to 1:2 | 50:1 to 2:1 | 37:1 to 5:1 |
| zoxamide | 6:1 to 1:18 | 2:1 to 1:6 | 2:1 to 1:4 |
| 5-chloro-6-(2,4,6-trifluorophenyl)- | 15:1 to 1:36 | 5:1 to 1:12 | 1:1 to 1:6 |
| 7-(4-methylpiperidin-1-yl)[1,2,4]triazolo- | | | |
| [1,5-a]pyrimidine (DPX-BAS600F) | | | |
| N-[2-[4-[[3-(4-chlorophenyl)-2-propyn- | 6:1 to 1:18 | 2:1 to 1:6 | 2:1 to 1:4 |
| 1-yl]oxy]-3-methoxyphenyl]ethyl]- | | | |
| 3-methyl-2-[(methylsulfonyl)amino]- | | | |
| butanamide | | | |
| N-[2-[4-[[3-(4-chlorophenyl)-2-propyn-1- | 6:1 to 1:18 | 2:1 to 1:6 | 2:1 to 1:4 |
| yl]oxy]-3-methoxyphenyl]ethyl]-3-methyl- | | | |
| 2-[(ethylsulfonyl)amino]butanamide | | | |
| N′-[4-[[3-[(4-chlorophenyl)methyl]-1,2,4- | 20:1 to 1:20 | 8:1 to 1:8 | 3:1 to 1:3 |
| thiadiazol-5-yl]oxy]-2,5-dimethylphenyl]- | | | |
| N-ethyl-N-methylmethanimidamide | | | |
| 4-fluorophenyl N-[1-[[[1-(4-cyanophenyl)- | 6:1 to 1:18 | 2:1 to 1:6 | 2:1 to 1:4 |
| ethyl]sulfonyl]methyl]propyl]carbamate | | | |
| N-[[(cyclopropylmethoxy)amino][6- | 1:1 to 1:90 | 1:2 to 1:30 | 1:2 to 1:24 |
| (difluoromethoxy)-2,3-difluorophenyl]- | | | |
| methylene]benzeneacetamide | | | |
| α-[methoxyimino]-N-methyl-2-[[[1-[3- | 9:1 to 1:18 | 3:1 to 1:6 | 3:1 to 1:3 |
| (trifluoromethyl)phenyl]ethoxy]imino]- | | | |
| methyl]benzeneacetamide | | | |
| N-[4-[4-chloro-3-(trifluoromethyl)- | 15:1 to 1:18 | 5:1 to 1:6 | 3:1 to 1:3 |
| phenoxy]-2,5-dimethylphenyl]-N-ethyl- | | | |
| N-methylmethanimidamide | | | |
| pentyl N-[4-[[[[(1-methyl-1H-tetrazol- | 9:1 to 1:18 | 3:1 to 1:6 | 3:1 to 1:3 |
| 5-yl)phenylmethylene]amino]oxy]methyl]- | | | |
| 2-thiazolyl]carbamate | | | |
| pentyl N-[6-[[[[(1-methyl-1H-tetrazol- | 9:1 to 1:18 | 3:1 to 1:6 | 3:1 to 1:3 |
| 5-yl)phenylmethylene]amino]oxy]methyl]- | | | |
| 2-pyridinyl]carbamate | | | |
| 2-[(3-bromo-6-quinolinyl)oxy]- | 5:1 to 1:22 | 2:1 to 1:8 | 2:1 to 1:4 |
| N-(1,1-dimethyl-2-butyn-1-yl)- | | | |
| 2-(methylthio)acetamide | | | |
| 2-[(3-ethynyl-6-quinolinyl)oxy]- | 5:1 to 1:22 | 2:1 to 1:8 | 2:1 to 1:4 |
| N-[1-(hydroxymethyl)-1-methyl-2-propyn- | | | |
| 1-yl]-2-(methylthio)acetamide | | | |
| N-(1,1-dimethyl-2-butyn-1-yl)-2-[(3- | 5:1 to 1:22 | 2:1 to 1:8 | 2:1 to 1:4 |
| ethynyl-6-quinolinyl)oxy]-2- | | | |
| (methylthio)acetamide | | | |

As already noted, the present invention includes embodiments wherein in the composition comprising components (a) and (b), wherein component (b) comprises at least one fungicidal compound from each of two groups selected from (b) through (b54). Table C1 list specific mixtures to illustrate embodiments wherein component (b) includes at least one fungicidal compound from each of two groups selected from (b3) through (b54). Table C1 discloses a mixture of Compound 1 of the present invention with at least two Component (b) compounds. The entries under the heading “Illustrative Ratios” disclose three specific weight ratios of Component (a) to each Component (b) compound. For example, the first line discloses a mixture of Compound 1 with cyproconazole and azoxystrobin and lists weight ratios of Compound 1 to cyproconazole to azoxystrobin of 1:1:1, 2:1:1 or 3:1:1.

TABLE C1

| Component (a) | Component (b) | | | Illustrative Ratios(*) | | |
|---|---|---|---|---|---|---|
| Compound 1 | cyproconazole | azoxystrobin | | 1:1:1 | 2:1:1 | 3:1:1 |
| Compound 1 | cyproconazole | kresoxim-methyl | | 1:1:1 | 2:1:1 | 3:1:1 |
| Compound 1 | cyproconazole | picoxystrobin | | 1:1:1 | 2:1:1 | 3:1:1 |
| Compound 1 | cyproconazole | pyraclostrobin | | 1:1:1 | 2:1:1 | 3:1:1 |
| Compound 1 | cyproconazole | trifloxystrobin | | 1:1:1 | 2:1:1 | 3:1:1 |
| Compound 1 | cyproconazole | bixafen | | 1:1:2 | 2:1:2 | 3:1:2 |
| Compound 1 | cyproconazole | boscalid | | 1:1:2 | 2:1:2 | 3:1:2 |
| Compound 1 | cyproconazole | cyflufenamid | | 1:2:1 | 2:2:1 | 3:2:1 |
| Compound 1 | cyproconazole | fluopyram | | 1:1:2 | 2:1:2 | 3:1:2 |
| Compound 1 | cyproconazole | isopyrazam | | 1:1:2 | 2:1:2 | 3:1:2 |
| Compound 1 | cyproconazole | metrafenone | | 1:1:2 | 2:1:2 | 3:1:2 |
| Compound 1 | cyproconazole | penthiopyrad | | 1:1:2 | 2:1:2 | 3:1:2 |
| Compound 1 | cyproconazole | proquinazid | | 1:1:1 | 2:1:1 | 3:1:1 |
| Compound 1 | cyproconazole | pyriofenone | | 1:1:2 | 2:1:2 | 3:1:2 |
| Compound 1 | cyproconazole | quinoxyfen | | 1:1:1 | 2:1:1 | 3:1:1 |
| Compound 1 | cyproconazole | sedaxane | | 1:1:2 | 2:1:2 | 3:1:2 |
| Compound 1 | cyproconazole | picoxystrobin | proquinazid | 1:1:1:1 | 2:1:1:1 | 3:1:1:1 |
| Compound 1 | cyproconazole | trifloxystrobin | proquinazid | 1:1:1:1 | 2:1:1:1 | 3:1:1:1 |
| Compound 1 | difenconazole | azoxystrobin | | 1:1:1 | 2:1:1 | 3:1:1 |
| Compound 1 | difenconazole | kresoxim-methyl | | 1:1:1 | 2:1:1 | 3:1:1 |
| Compound 1 | difenconazole | picoxystrobin | | 1:1:1 | 2:1:1 | 3:1:1 |
| Compound 1 | difenconazole | pyraclostrobin | | 1:1:1 | 2:1:1 | 3:1:1 |
| Compound 1 | difenconazole | pyrametostrobin | | 1:1:1 | 2:1:1 | 3:1:1 |
| Compound 1 | difenconazole | pyraoxystrobin | | 1:1:1 | 2:1:1 | 3:1:1 |
| Compound 1 | difenconazole | trifloxystrobin | | 1:1:1 | 2:1:1 | 3:1:1 |
| Compound 1 | difenconazole | bixafen | | 1:1:2 | 2:1:2 | 3:1:2 |
| Compound 1 | difenconazole | boscalid | | 1:1:2 | 2:1:2 | 3:1:2 |
| Compound 1 | difenconazole | cyflufenamid | | 1:2:1 | 2:2:1 | 3:2:1 |
| Compound 1 | difenconazole | fluopyram | | 1:1:2 | 2:1:2 | 3:1:2 |
| Compound 1 | difenconazole | isopyrazam | | 1:1:2 | 2:1:2 | 3:1:2 |
| Compound 1 | difenconazole | metrafenone | | 1:1:2 | 2:1:2 | 3:1:2 |
| Compound 1 | difenconazole | penthiopyrad | | 1:1:2 | 2:1:2 | 3:1:2 |
| Compound 1 | difenconazole | proquinazid | | 1:1:1 | 2:1:1 | 3:1:1 |
| Compound 1 | difenconazole | pyriofenone | | 1:1:2 | 2:1:2 | 3:1:2 |
| Compound 1 | difenconazole | quinoxyfen | | 1:1:1 | 2:1:1 | 3:1:1 |
| Compound 1 | difenconazole | sedaxane | | 1:1:2 | 2:1:2 | 3:1:2 |
| Compound 1 | difenconazole | picoxystrobin | proquinazid | 1:1:1:1 | 2:1:1:1 | 3:1:1:1 |
| Compound 1 | difenconazole | trifloxystrobin | proquinazid | 1:1:1:1 | 2:1:1:1 | 3:1:1:1 |
| Compound 1 | epoxiconazole | azoxystrobin | | 1:1:1 | 2:1:1 | 3:1:1 |
| Compound 1 | epoxiconazole | kresoxim-methyl | | 1:1:1 | 2:1:1 | 3:1:1 |
| Compound 1 | epoxiconazole | picoxystrobin | | 1:1:1 | 2:1:1 | 3:1:1 |
| Compound 1 | epoxiconazole | pyraclostrobin | | 1:1:1 | 2:1:1 | 3:1:1 |
| Compound 1 | difenconazole | pyrameotostrobin | | 1:1:1 | 2:1:1 | 3:1:1 |
| Compound 1 | difenconazole | pyraoxystrobin | | 1:1:1 | 2:1:1 | 3:1:1 |
| Compound 1 | epoxiconazole | trifloxystrobin | | 1:1:1 | 2:1:1 | 3:1:1 |
| Compound 1 | epoxiconazole | bixafen | | 1:1:2 | 2:1:2 | 3:1:2 |
| Compound 1 | epoxiconazole | boscalid | | 1:1:2 | 2:1:2 | 3:1:2 |
| Compound 1 | epoxiconazole | cyflufenamid | | 1:2:1 | 2:2:1 | 3:2:1 |
| Compound 1 | epoxiconazole | fluopyram | | 1:1:2 | 2:1:2 | 3:1:2 |
| Compound 1 | epoxiconazole | isopyrazam | | 1:1:2 | 2:1:2 | 3:1:2 |
| Compound 1 | epoxiconazole | metrafenone | | 1:1:2 | 2:1:2 | 3:1:2 |
| Compound 1 | epoxiconazole | penthiopyrad | | 1:1:2 | 2:1:2 | 3:1:2 |
| Compound 1 | epoxiconazole | proquinazid | | 1:1:1 | 2:1:1 | 3:1:1 |
| Compound 1 | epoxiconazole | pyriofenone | | 1:1:2 | 2:1:2 | 3:1:2 |
| Compound 1 | epoxiconazole | quinoxyfen | | 1:1:1 | 2:1:1 | 3:1:1 |
| Compound 1 | epoxiconazole | sedaxane | | 1:1:2 | 2:1:2 | 3:1:2 |
| Compound 1 | epoxiconazole | picoxystrobin | proquinazid | 1:1:1:1 | 2:1:1:1 | 3:1:1:1 |
| Compound 1 | epoxiconazole | trifloxystrobin | proquinazid | 1:1:1:1 | 2:1:1:1 | 3:1:1:1 |
| Compound 1 | metconazole | azoxystrobin | | 1:1:1 | 2:1:1 | 3:1:1 |
| Compound 1 | metconazole | kresoxim-methyl | | 1:1:1 | 2:1:1 | 3:1:1 |
| Compound 1 | metconazole | picoxystrobin | | 1:1:1 | 2:1:1 | 3:1:1 |
| Compound 1 | metconazole | pyraclostrobin | | 1:1:1 | 2:1:1 | 3:1:1 |
| Compound 1 | metconazole | pyrametostrobin | | 1:1:1 | 2:1:1 | 3:1:1 |
| Compound 1 | metconazole | pyraoxystrobin | | 1:1:1 | 2:1:1 | 3:1:1 |
| Compound 1 | metconazole | trifloxystrobin | | 1:1:1 | 2:1:1 | 3:1:1 |
| Compound 1 | metconazole | bixafen | | 1:1:2 | 2:1:2 | 3:1:2 |
| Compound 1 | metconazole | boscalid | | 1:1:2 | 2:1:2 | 3:1:2 |
| Compound 1 | metconazole | cyflufenamid | | 1:2:1 | 2:2:1 | 3:2:1 |
| Compound 1 | metconazole | fluopyram | | 1:1:2 | 2:1:2 | 3:1:2 |
| Compound 1 | metconazole | isopyrazam | | 1:1:2 | 2:1:2 | 3:1:2 |
| Compound 1 | metconazole | metrafenone | | 1:1:2 | 2:1:2 | 3:1:2 |
| Compound 1 | metconazole | penthiopyrad | | 1:1:2 | 2:1:2 | 3:1:2 |
| Compound 1 | metconazole | proquinazid | | 1:1:1 | 2:1:1 | 3:1:1 |
| Compound 1 | metconazole | pyriofenone | | 1:1:2 | 2:1:2 | 3:1:2 |
| Compound 1 | metconazole | quinoxyfen | | 1:1:1 | 2:1:1 | 3:1:1 |
| Compound 1 | metconazole | sedaxane | | 1:1:2 | 2:1:2 | 3:1:2 |
| Compound 1 | metconazole | picoxystrobin | proquinazid | 1:1:1:1 | 2:1:1:1 | 3:1:1:1 |
| Compound 1 | metconazole | trifloxystrobin | proquinazid | 1:1:1:1 | 2:1:1:1 | 3:1:1:1 |
| Compound 1 | myclobutanil | azoxystrobin | | 1:1:1 | 2:1:1 | 3:1:1 |
| Compound 1 | myclobutanil | kresoxim-methyl | | 1:1:1 | 2:1:1 | 3:1:1 |
| Compound 1 | myclobutanil | picoxystrobin | | 1:1:1 | 2:1:1 | 3:1:1 |
| Compound 1 | myclobutanil | pyraclostrobin | | 1:1:1 | 2:1:1 | 3:1:1 |
| Compound 1 | myclobutanil | pyrametostrobin | | 1:1:1 | 2:1:1 | 3:1:1 |
| Compound 1 | myclobutanil | pyraoxystrobin | | 1:1:1 | 2:1:1 | 3:1:1 |
| Compound 1 | myclobutanil | trifloxystrobin | | 1:1:1 | 2:1:1 | 3:1:1 |
| Compound 1 | myclobutanil | bixafen | | 1:1:2 | 2:1:2 | 3:1:2 |
| Compound 1 | myclobutanil | boscalid | | 1:1:2 | 2:1:2 | 3:1:2 |
| Compound 1 | myclobutanil | cyflufenamid | | 1:2:1 | 2:2:1 | 3:2:1 |
| Compound 1 | myclobutanil | fluopyram | | 1:1:2 | 2:1:2 | 3:1:2 |
| Compound 1 | myclobutanil | isopyrazam | | 1:1:2 | 2:1:2 | 3:1:2 |
| Compound 1 | myclobutanil | metrafenone | | 1:1:2 | 2:1:2 | 3:1:2 |
| Compound 1 | myclobutanil | penthiopyrad | | 1:1:2 | 2:1:2 | 3:1:2 |
| Compound 1 | myclobutanil | proquinazid | | 1:1:1 | 2:1:1 | 3:1:1 |
| Compound 1 | myclobutanil | pyriofenone | | 1:1:2 | 2:1:2 | 3:1:2 |
| Compound 1 | myclobutanil | quinoxyfen | | 1:1:1 | 2:1:1 | 3:1:1 |
| Compound 1 | myclobutanil | sedaxane | | 1:1:2 | 2:1:2 | 3:1:2 |
| Compound 1 | myclobutanil | picoxystrobin | proquinazid | 1:1:1:1 | 2:1:1:1 | 3:1:1:1 |
| Compound 1 | myclobutanil | trifloxystrobin | proquinazid | 1:1:1:1 | 2:1:1:1 | 3:1:1:1 |
| Compound 1 | prothioconazole | azoxystrobin | | 1:1:1 | 2:1:1 | 3:1:1 |
| Compound 1 | prothioconazole | kresoxim-methyl | | 1:1:1 | 2:1:1 | 3:1:1 |
| Compound 1 | prothioconazole | picoxystrobin | | 1:1:1 | 2:1:1 | 3:1:1 |
| Compound 1 | prothioconazole | pyraclostrobin | | 1:1:1 | 2:1:1 | 3:1:1 |
| Compound 1 | prothioconazole | pyrametostrobin | | 1:1:1 | 2:1:1 | 3:1:1 |
| Compound 1 | prothioconazole | pyraoxystrobin | | 1:1:1 | 2:1:1 | 3:1:1 |
| Compound 1 | prothioconazole | trifloxystrobin | | 1:1:1 | 2:1:1 | 3:1:1 |
| Compound 1 | prothioconazole | bixafen | | 1:1:2 | 2:1:2 | 3:1:2 |
| Compound 1 | prothioconazole | boscalid | | 1:1:2 | 2:1:2 | 3:1:2 |
| Compound 1 | prothioconazole | cyflufenamid | | 1:2:1 | 2:2:1 | 3:2:1 |
| Compound 1 | prothioconazole | fluopyram | | 1:1:2 | 2:1:2 | 3:1:2 |
| Compound 1 | prothioconazole | isopyrazam | | 1:1:2 | 2:1:2 | 3:1:2 |
| Compound 1 | prothioconazole | metrafenone | | 1:1:2 | 2:1:2 | 3:1:2 |
| Compound 1 | prothioconazole | penthiopyrad | | 1:1:2 | 2:1:2 | 3:1:2 |
| Compound 1 | prothioconazole | proquinazid | | 1:1:1 | 2:1:1 | 3:1:1 |
| Compound 1 | prothioconazole | pyriofenone | | 1:1:2 | 2:1:2 | 3:1:2 |
| Compound 1 | prothioconazole | quinoxyfen | | 1:1:1 | 2:1:1 | 3:1:1 |
| Compound 1 | prothioconazole | sedaxane | | 1:1:2 | 2:1:2 | 3:1:2 |
| Compound 1 | prothioconazole | picoxystrobin | proquinazid | 1:1:1:1 | 2:1:1:1 | 3:1:1:1 |
| Compound 1 | prothioconazole | trifloxystrobin | proquinazid | 1:1:1:1 | 2:1:1:1 | 3:1:1:1 |
| Compound 1 | tebuconazole | azoxystrobin | | 1:1:1 | 2:1:1 | 3:1:1 |
| Compound 1 | tebuconazole | kresoxim-methyl | | 1:1:1 | 2:1:1 | 3:1:1 |
| Compound 1 | tebuconazole | picoxystrobin | | 1:1:1 | 2:1:1 | 3:1:1 |
| Compound 1 | tebuconazole | pyraclostrobin | | 1:1:1 | 2:1:1 | 3:1:1 |
| Compound 1 | tebuconazole | pyrametostrobin | | 1:1:1 | 2:1:1 | 3:1:1 |
| Compound 1 | tebuconazole | pyraoxystrobin | | 1:1:1 | 2:1:1 | 3:1:1 |
| Compound 1 | tebuconazole | trifloxystrobin | | 1:1:1 | 2:1:1 | 3:1:1 |
| Compound 1 | tebuconazole | bixafen | | 1:1:2 | 2:1:2 | 3:1:2 |
| Compound 1 | tebuconazole | boscalid | | 1:1:2 | 2:1:2 | 3:1:2 |
| Compound 1 | tebuconazole | cyflufenamid | | 1:2:1 | 2:2:1 | 3:2:1 |
| Compound 1 | tebuconazole | fluopyram | | 1:1:2 | 2:1:2 | 3:1:2 |
| Compound 1 | tebuconazole | isopyrazam | | 1:1:2 | 2:1:2 | 3:1:2 |
| Compound 1 | tebuconazole | metrafenone | | 1:1:2 | 2:1:2 | 3:1:2 |
| Compound 1 | tebuconazole | penthiopyrad | | 1:1:2 | 2:1:2 | 3:1:2 |
| Compound 1 | tebuconazole | proquinazid | | 1:1:1 | 2:1:1 | 3:1:1 |
| Compound 1 | tebuconazole | pyriofenone | | 1:1:2 | 2:1:2 | 3:1:2 |
| Compound 1 | tebuconazole | quinoxyfen | | 1:1:1 | 2:1:1 | 3:1:1 |
| Compound 1 | tebuconazole | sedaxane | | 1:1:2 | 2:1:2 | 3:1:2 |
| Compound 1 | tebuconazole | picoxystrobin | proquinazid | 1:1:1:1 | 2:1:1:1 | 3:1:1:1 |
| Compound 1 | tebuconazole | trifloxystrobin | proquinazid | 1:1:1:1 | 2:1:1:1 | 3:1:1:1 |

(*)Ratios of Component (a) relative to Component (b) in sequence, by weight.

Tables C2 through C15 are each constructed the same as Table C1 above except that entries below the “Component (a)” column heading are replaced with the respective Component (a) Column Entry shown below. Thus, for example, in Table C2 the entries below the “Component (a)” column heading all recite “Compound 18”. Therefore, the first entry in Table C2 specifically discloses a mixture of Compound 18 with cyproconazole and azoxystrobin, with the illustrative weight ratios of 1:1:1, 2:1:1 and 3:1:1 of Compound 18 to cyproconazole to azoxystrobin. Tables C3 through C15 are constructed similarly.

| Table Number | Component (a) Column Entry |
|---|---|
| C2 | Compound 18 |
| C3 | Compound 19 |
| C4 | Compound 23 |
| C5 | Compound 57 |
| C6 | Compound 60 |
| C7 | Compound 68 |
| C8 | Compound 72 |
| C9 | Compound 73 |
| C10 | Compound 93 |
| C11 | Compound 111 |
| C12 | Compound 112 |
| C13 | Compound 118 |
| C14 | Compound 121 |
| C15 | Compound 127 |

Of note is a composition of the present invention comprising a compound of Formula 1 (or an N-oxide or salt thereof) with at least one other fungicidal compound that has a different site of action from the compound of Formula 1. In certain instances, a combination with at least one other fungicidal compound having a similar spectrum of control but a different site of action will be particularly advantageous for resistance management. Thus, a composition of the present invention can advantageously comprise at least one fungicidal active compound selected from the group consisting of (b1) through (b54) as described above, having a similar spectrum of control but a different site of action.

Compositions of component (a), or component (a) with component (b), can be further mixed with one or more other biologically active compounds or agents including insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of component (a), or a mixture of component (a) with component (b), and a biologically effective amount of at least one additional biologically active compound or agent and can further comprise at least one of a surfactant, a solid diluent or a liquid diluent.

The other biologically active compounds or agents can also be separately formulated in compositions comprising at least one of a surfactant, solid or liquid diluent. For compositions of the present invention, one or more other biologically active compounds or agents can be formulated together with one or both of components (a) and (b) to form a premix, or one or more other biologically active compounds or agents can be formulated separately from components (a) and (b) and the formulations combined together before application (e.g., in a spray tank) or, alternatively, applied in succession.

Examples of such biologically active compounds or agents with which compositions of component (a), or component (a) with component (b), can be formulated are: insecticides such as abamectin, acephate, acequinocyl, acetamiprid, acrinathrin, acynonapyr, afidopyropen, amidoflumet, amitraz, avermectin, azadirachtin, azinphos-methyl, benfuracarb, bensultap, benzpyrimoxan, bifenthrin, kappa-bifenthrin, bifenazate, bistrifluron, borate, broflanilide, buprofezin, cadusafos, carbaryl, carbofuran, cartap, carzol, chlorantraniliprole, chlorfenapyr, chlorfluazuron, chloroprallethrin, chlorpyrifos, chlorpyrifos-e, chlorpyrifos-methyl, chromafenozide, clofentezin, chloroprallethrin, clothianidin, cyantraniliprole, cyclaniliprole, cycloprothrin, cycloxaprid, cyenopyrafen, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyromazine, deltamethrin, diafenthiuron, diazinon, dicloromesotiaz, dieldrin, diflubenzuron, dimefluthrin, dimehypo, dimethoate, dimpropyridaz, dinotefuran, diofenolan, emamectin, emamectin benzoate, endosulfan, esfenvalerate, ethiprole, etofenprox, epsilon-metofluthrin, etoxazole, fenbutatin oxide, fenitrothion, fenothiocarb, fenoxycarb, fenpropathrin, fenvalerate, fipronil, flometoquin, flonicamid, fluazaindolizine, flubendiamide, flucythrinate, flufenerim, flufenoxuron, flufenoxystrobin, fluensulfone, fluhexafon, fluopyram, flupiprole, flupyradifurone, flupyrimin, fluvalinate, tau-fluvalinate, fluxametamide, fonophos, formetanate, fosthiazate, gamma-cyhalothrin, halofenozide, heptafluthrin, hexaflumuron, hexythiazox, hydramethylnon, imidacloprid, indoxacarb, insecticidal soaps, isofenphos, isocycloseram, kappa-tefluthrin, lambda-cyhalothrin, lufenuron, malathion, meperfluthrin, metaflumizone, metaldehyde, methamidophos, methidathion, methiocarb, methomyl, methoprene, methoxychlor, metofluthrin, methoxyfenozide, epsilon-metofluthrin, epsilon-momfluorothrin, monocrotophos, monofluorothrin, nicotine, nitenpyram, nithiazine, novaluron, noviflumuron, oxamyl, oxazosulfyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrin, pyridaben, pyridalyl, pyrifluquinazon, pyriminostrobin, pyriprole, pyriproxyfen, rotenone, ryanodine, silafluofen, spinetoram, spinosad, spirodiclofen, spiromesifen, spiropidion, spirotetramat, sulprofos, sulfoxaflor, tebufenozide, tebufenpyrad, teflubenzuron, tefluthrin, kappa-tefluthrin, terbufos, tetrachlorantraniliprole, tetrachlorvinphos, tetramethrin, tetramethylfluthrin, tetraniliprole, thiacloprid, thiamethoxam, thiodicarb, thiosultap-sodium, tioxazafen, tolfenpyrad, tralomethrin, triazamate, trichlorfon, triflumezopyrim, triflumuron, tyclopyrazoflor, zeta-cypermethrin, Bacillus thuringiensis delta-endotoxins, entomopathogenic bacteria, entomopathogenic viruses or entomopathogenic fungi.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the weight ratio of these various mixing partners (in total) to component (a), or a mixture of component (a) with component (b), is generally between about 1:3000 and about 3000:1. Of note are weight ratios between about 1:100 and about 3000:1, or between about 1:30 and about 300:1 (for example ratios between about 1:1 and about 30:1). It will be evident that including these additional components may expand the spectrum of diseases controlled beyond the spectrum controlled by component (a), or a mixture of component (a) with component (b).

Component (a) compounds and/or combinations thereof with component (b) compounds and/or one or more other biologically active compounds or agents can be applied to plants genetically transformed to express proteins toxic to invertebrate pests (such as Bacillus thuringiensis delta-endotoxins). The effect of the exogenously applied present component (a) alone or in combination with component (b) may be synergistic with the expressed toxin proteins.

Of note is the combination or the composition comprising component (a), or components (a) and (b), as described in the Summary of the Invention further comprising at least one invertebrate pest control compound or agent (e.g., insecticide, acaricide). Of particular note is a composition comprising component (a) and at least one (i.e. one or more) invertebrate pest control compound or agent, which then can be subsequently combined with component (b) to provide a composition comprising components (a) and (b) and the one or more invertebrate pest control compounds or agents. Alternatively without first mixing with component (b), a biologically effective amount of the composition comprising component (a) with at least one invertebrate pest control agent can be applied to a plant or plant seed (directly or through the environment of the plant or plant seed) to protect the plant or plant seed from diseases caused by fungal pathogens and injury caused by invertebrate pests.

Of note is a composition of the present invention which comprises in addition to a component (a) compound, alone or in combination with component (b), at least one invertebrate pest control compound or agent selected from the group consisting abamectin, acetamiprid, acrinathrin, acynonapyr, afidopyropen, amitraz, avermectin, azadirachtin, benfuracarb, bensultap, bifenthrin, buprofezin, broflanilide, cadusafos, carbaryl, cartap, chlorantraniliprole, chloroprallethrin, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, epsilon-metofluthrin, esfenvalerate, ethiprole, etofenprox, etoxazole, fenitrothion, fenothiocarb, fenoxycarb, fenvalerate, fipronil, flometoquin, fluxametamide, flonicamid, flubendiamide, fluensulfone, flufenoxuron, flufenoxystrobin, flufensulfone, flupiprole, flupyrimin, flupyradifurone, fluvalinate, formetanate, fosthiazate, gamma-cyhalothrin, heptafluthrin, hexaflumuron, hydramethylnon, imidacloprid, indoxacarb, isocycloseram, kappa-tefluthrin, lambda-cyhalothrin, lufenuron, meperfluthrin, metaflumizone, methiodicarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, tyclopyrazoflor, zeta-cypermethrin, Bacillus thuringiensis delta-endotoxins, all strains of Bacillus thuringiensis and all strains of nucleo polyhedrosis viruses.

In certain instances, combinations of a a component (a) compound of this invention, alone or in mixture with component (b), with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When an enhanced effect of fungicidal active ingredients occurs at application rates giving agronomically satisfactory levels of fungal control, such combinations can be advantageous for reducing crop production cost and decreasing environmental load.

Table D1 lists specific combinations of invertebrate pest control agents with Compound 1 (compound numbers refer to compounds in Index Tables A-B) as a component (a) compound illustrative of mixtures and compositions comprising these active ingredients and methods using them according to the present invention. The second column of Table D1 lists the specific invertebrate pest control agents (e.g., “Abamectin” in the first line). The third column of Table D1 lists the mode of action (if known) or chemical class of the invertebrate pest control agents. The fourth column of Table D1 lists embodiment(s) of ranges of weight ratios for rates at which the invertebrate pest control agent is typically applied relative to Compound 1 alone or in combination with component (b) (e.g., “50:1 to 1:50” of abamectin relative to a Compound 1 by weight). Thus, for example, the first line of Table D1 specifically discloses the combination of Compound 1 with abamectin is typically applied in a weight ratio between 50:1 to 1:50. The remaining lines of Table D1 are to be construed similarly.

TABLE D1

| | Invertebrate Pest Control | Mode of Action or Chemical | Typical |
|---|---|---|---|
| Component (a) | Agent | Class | Weight Ratio |
| Compound 1 | Abamectin | macrocyclic lactones | 50:1 to 1:50 |
| Compound 1 | Acetamiprid | neonicotinoids | 150:1 to 1:200 |
| Compound 1 | Amitraz | octopamine receptor ligands | 200:1 to 1:100 |
| Compound 1 | Avermectin | macrocyclic lactones | 50:1 to 1:50 |
| Compound 1 | Azadirachtin | ecdysone agonists | 100:1 to 1:120 |
| Compound 1 | Beta-cyfluthrin | sodium channel modulators | 150:1 to 1:200 |
| Compound 1 | Bifenthrin | sodium channel modulators | 100:1 to 1:10 |
| Compound 1 | Buprofezin | chitin synthesis inhibitors | 500:1 to 1:50 |
| Compound 1 | Cartap | nereistoxin analogs | 100:1 to 1:200 |
| Compound 1 | Chlorantraniliprole | ryanodine receptor ligands | 100:1 to 1:120 |
| Compound 1 | Chlorfenapyr | mitochondrial electron transport | 300:1 to 1:200 |
| | | inhibitors | |
| Compound 1 | Chlorpyrifos | cholinesterase inhibitors | 500:1 to 1:200 |
| Compound 1 | Clothianidin | neonicotinoids | 100:1 to 1:400 |
| Compound 1 | Cyantraniliprole | ryanodine receptor ligands | 100:1 to 1:120 |
| Compound 1 | Cyfluthrin | sodium channel modulators | 150:1 to 1:200 |
| Compound 1 | Cyhalothrin | sodium channel modulators | 150:1 to 1:200 |
| Compound 1 | Cypermethrin | sodium channel modulators | 150:1 to 1:200 |
| Compound 1 | Cyromazine | chitin synthesis inhibitors | 400:1 to 1:50 |
| Compound 1 | Deltamethrin | sodium channel modulators | 50:1 to 1:400 |
| Compound 1 | Dieldrin | cyclodiene insecticides | 200:1 to 1:100 |
| Compound 1 | Dinotefuran | neonicotinoids | 150:1 to 1:200 |
| Compound 1 | Diofenolan | molting inhibitor | 150:1 to 1:200 |
| Compound 1 | Emamectin | macrocyclic lactones | 50:1 to 1:10 |
| Compound 1 | Endosulfan | cyclodiene insecticides | 200:1 to 1:100 |
| Compound 1 | Esfenvalerate | sodium channel modulators | 100:1 to 1:400 |
| Compound 1 | Ethiprole | GABA-regulated chloride channel | 200:1 to 1:100 |
| | | blockers | |
| Compound 1 | Fenothiocarb | | 150:1 to 1:200 |
| Compound 1 | Fenoxycarb | juvenile hormone mimics | 500:1 to 1:100 |
| Compound 1 | Fenvalerate | sodium channel modulators | 150:1 to 1:200 |
| Compound 1 | Fipronil | GABA-regulated chloride channel | 150:1 to 1:100 |
| | | blockers | |
| Compound 1 | Flonicamid | | 200:1 to 1:100 |
| Compound 1 | Flubendiamide | ryanodine receptor ligands | 100:1 to 1:120 |
| Compound 1 | Flufenoxuron | chitin synthesis inhibitors | 200:1 to 1:100 |
| Compound 1 | Hexaflumuron | chitin synthesis inhibitors | 300:1 to 1:50 |
| Compound 1 | Hydramethylnon | mitochondrial electron transport | 150:1 to 1:250 |
| | | inhibitors | |
| Compound 1 | Imidacloprid | neonicotinoids | 1000:1 to 1:1000 |
| Compound 1 | Indoxacarb | sodium channel modulators | 200:1 to 1:50 |
| Compound 1 | Lambda-cyhalothrin | sodium channel modulators | 50:1 to 1:250 |
| Compound 1 | Lufenuron | chitin synthesis inhibitors | 500:1 to 1:250 |
| Compound 1 | Meperfluthrin | Sodium channel modulators | 100:1 to 1:400 |
| Compound 1 | Metaflumizone | | 200:1 to 1:200 |
| Compound 1 | Methomyl | cholinesterase inhibitors | 500:1 to 1:100 |
| Compound 1 | Methoprene | juvenile hormone mimics | 500:1 to 1:100 |
| Compound 1 | Methoxyfenozide | ecdysone agonists | 50:1 to 1:50 |
| Compound 1 | Nitenpyram | neonicotinoids | 150:1 to 1:200 |
| Compound 1 | Nithiazine | neonicotinoids | 150:1 to 1:200 |
| Compound 1 | Novaluron | chitin synthesis inhibitors | 500:1 to 1:150 |
| Compound 1 | Oxamyl | cholinesterase inhibitors | 200:1 to 1:200 |
| Compound 1 | Pymetrozine | | 200:1 to 1:100 |
| Compound 1 | Pyrethrin | sodium channel modulators | 100:1 to 1:10 |
| Compound 1 | Pyridaben | mitochondrial electron transport | 200:1 to 1:100 |
| | | inhibitors | |
| Compound 1 | Pyridalyl | | 200:1 to 1:100 |
| Compound 1 | Pyriproxyfen | juvenile hormone mimics | 500:1 to 1:100 |
| Compound 1 | Ryanodine | ryanodine receptor ligands | 100:1 to 1:120 |
| Compound 1 | Spinetoram | macrocyclic lactones | 150:1 to 1:100 |
| Compound 1 | Spinosad | macrocyclic lactones | 500:1 to 1:10 |
| Compound 1 | Spirodiclofen | lipid biosynthesis inhibitors | 200:1 to 1:200 |
| Compound 1 | Spiromesifen | lipid biosynthesis inhibitors | 200:1 to 1:200 |
| Compound 1 | Sulfoxaflor | | 200:1 to 1:200 |
| Compound 1 | Tebufenozide | ecdysone agonists | 500:1 to 1:250 |
| Compound 1 | Tetramethylfluthrin | Sodium channel modulators | 100:1 to 1:400 |
| Compound 1 | Thiacloprid | neonicotinoids | 100:1 to 1:200 |
| Compound 1 | Thiamethoxam | neonicotinoids | 1250:1 to 1:1000 |
| Compound 1 | Thiodicarb | cholinesterase inhibitors | 500:1 to 1:400 |
| Compound 1 | Thiosultap-sodium | | 150:1 to 1:100 |
| Compound 1 | Tralomethrin | sodium channel modulators | 150:1 to 1:200 |
| Compound 1 | Triazamate | cholinesterase inhibitors | 250:1 to 1:100 |
| Compound 1 | Triflumuron | chitin synthesis inhibitors | 200:1 to 1:100 |
| Compound 1 | Bacillus thuringiensis | biological agents | 50:1 to 1:10 |
| Compound 1 | Bacillus thuringiensis delta- | biological agents | 50:1 to 1:10 |
| | endotoxin | | |
| Compound 1 | NPV (e.g., Gemstar) | biological agents | 50:1 to 1:10 |

Tables D2 through D15 are each constructed the same as Table D1 above except that entries below the “Component (a)” column heading are replaced with the respective Component (a) Column Entry shown below. Thus, for example, in Table D2 the entries below the “Component (a)” column heading all recite “Compound 18”, and the first line below the column headings in Table D2 specifically discloses a mixture of Compound 18 with abamectin. Tables D3 through D15 are constructed similarly.

| Table Number | Component (a) Column Entry |
|---|---|
| D2 | Compound 18 |
| D3 | Compound 19 |
| D4 | Compound 23 |
| D5 | Compound 57 |
| D6 | Compound 60 |
| D7 | Compound 68 |
| D8 | Compound 72 |
| D9 | Compound 73 |
| D10 | Compound 93 |
| D11 | Compound 111 |
| D12 | Compound 112 |
| D13 | Compound 118 |
| D14 | Compound 121 |
| D15 | Compound 127 |

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM I-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize™ AG.

Seed treatments can also include one or more plant activators that induce systemic acquired resistance in plants following contact by a pathogen. An example of a plant activator which induces such protective mechanisms is acibenzolar-S-methyl.

In the present fungicidal compositions, the Formula 1 compounds of component (a) can work synergically with the additional fungicidal compounds of component (b) to provide such beneficial results as broadening the spectrum of plant diseases controlled, extending duration of preventative and curative protection, and suppressing proliferation of resistant fungal pathogens. In particular embodiments, compositions are provided in accordance with this invention that comprise proportions of component (a) and component (b) that are especially useful for controlling particular fungal diseases (such as Alternaria solani, Blumeria graminis f. sp. tritici, Botrytis cinerea, Puccinia recondita f. sp. tritici, Rhizoctonia solani, Septoria nodorum, Septoria tritici).

Mixtures of fungicides may also provide significantly better disease control than could be predicted based on the activity of the individual components. This synergism has been described as “the cooperative action of two components of a mixture, such that the total effect is greater or more prolonged than the sum of the effects of the two (or more) taken independently” (see P. M. L. Tames, Neth. J. Plant Pathology 1964, 70, 73-80). In methods providing plant disease control in which synergy is exhibited from a combination of active ingredients (e.g., fungicidal compounds) applied to the plant or seed, the active ingredients are applied in a synergistic weight ratio and synergistic (i.e. synergistically effective) amounts. Measures of disease control, inhibition and prevention cannot exceed 100%. Therefore expression of substantial synergism typically requires use of application rates of active ingredients wherein the active ingredients separately provide much less than 100% effect, so that their additive effect is substantially less than 100% to allow the possibility of increase in effect as result of synergism. On the other hand, application rates of active ingredients that are too low may show not show much activity in mixtures even with the benefit of synergism. One skilled in the art can easily identify and optimize through simple experimentation the weight ratios and application rates (i.e. amounts) of fungicidal compounds providing synergy.

The presence of a synergistic effect between two active ingredients was established with the aid of the Colby equation (see Colby, S. R. “Calculating Synergistic and Antagonistic Responses of Herbicide Combinations”, Weeds, (1967), 15, 20-22):

$$p = A + B - [ \frac{A \times B}{100} ] .$$

Using the method of Colby, the presence of a synergistic interaction between two active ingredients is established by first calculating the predicted activity, p, of the mixture based on activities of the two components applied alone. If p is lower than the experimentally established effect, synergism has occurred. In the equation above, A is the fungicidal activity in percentage control of one component applied alone at rate x. The B term is the fungicidal activity in percentage control of the second component applied at rate y. The equation estimates p, the expected fungicidal activity of the mixture of A at rate x with B at rate y if their effects are strictly additive and no interaction has occurred.

Seed treatments can also include one or more plant activators that induce systemic acquired resistance in plants following contact by a pathogen. An example of a plant activator which induces such protective mechanisms is acibenzolar-S-methyl.

The following TESTS demonstrate the control efficacy of compounds of this invention on specific pathogens. The pathogen control protection afforded by the compounds is not limited, however, to these species. See Index Tables A-B below for compound descriptions. The following abbreviations are used in Index Table A: Me means methyl, i-Pr means iso-propyl, MeO means methoxy and —NO₂ means nitro. The abbreviation “Cmpd.” stands for “Compound”, and the abbreviation “Ex.” stands for “Example” and is followed by a number indicating in which example the compound is prepared. In Index Table A, the locant numbers listed for substituents R⁴ and R⁵ are as indicated in the structure above the table. The order of listing substituents R⁴ and R⁵ may be different from the Chemical Abstracts naming system if the difference does not affect the meaning. For example, Compound 1 in Index Table A lists the substituent R⁵ is at the 6-position (i.e. 6-F) whereas the CAS name for Compound 1 is 4-(2-bromo-4,6-difluorophenyl)-N-(2-fluoro-6-nitrophenyl)-1,3-dimethyl-1H-pyrazol-5-amine. The numerical value reported in the column “AP⁺ (M+1)”, is the molecular weight of the observed molecular ion formed by addition of H⁺ (molecular weight of 1) to the molecule having the greatest isotopic abundance (i.e. M); the numerical value reported in the column “AP⁻ (M−1)”, is the molecular weight of the observed molecular ion formed by loss of H⁺ (molecular weight of 1) from the molecule having the greatest isotopic abundance (i.e. M). The presence of molecular ions containing one or more higher atomic weight isotopes of lower abundance (e.g., ³⁷Cl, ⁸¹Br) is not reported. The reported M+1 and M−1 peaks were observed by mass spectrometry using electrospray ionization (ESI) or atmospheric pressure chemical ionization (APCI).

INDEX TABLE A

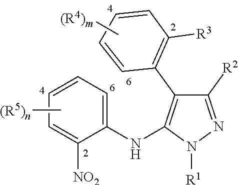

| | | | | | | m.p. | AP⁺ | AP⁻ |
|---|---|---|---|---|---|---|---|---|
| Cmpd. No. | R¹ | R² | R³ | (R⁴)ₘ | (R⁵)ₙ | (° C.) | (M + 1) | (M − 1) |
| 1 | CH₃ | CH₃ | Br | 4,6-di-F | 6-F | 133-137 | 442 | |
| 2 | CH₃ | CH₃ | F | 4-CH₃O, 6-F | 4-F, 6-Cl | 176-180 | 427 | |
| 3 | CH₃ | Et | F | 4-F | 4-Br, 6-Cl | 148-149 | 437 | |
| 4 | CH₃ | CH₃ | Cl | 4-F | 4-MeOCH₂ | 106-110 | 405 | |
| 5 | CH₃ | CH₃ | Cl | 4-F | 4-CH≡CCH₂O | 123-127 | 415 | |
| 6 | CH₃ | CH₃ | Cl | 4-F | 4-CH₃CH₂ | 109-113 | 389 | |
| 7 | CH₃ | CH₃ | Cl | 4-F | 5,6-di-F | 131-135 | 397 | |
| 8 | CH₃ | CH₃ | Cl | 4-F | 4-Br, 6-Cl | 180-184 | 475 | |
| 9 | CH₃ | CH₃ | Cl | 4-Cl | 4,6-di-F | 138-142 | 414 | |
| 10 | CH₃ | CH₃ | Cl | 4-Cl | 4-F, 6-Cl | 178-182 | 430 | |
| 11 | CH₃ | CH₃ | Cl | 4-Cl | 4-Cl, 6-F | 164-168 | 430 | |
| 12 | CH₃ | CH₃ | F | 4-Cl | 6-F | 151-155 | 379 | |
| 13 | CH₃ | CH₃ | F | 4-Cl | 4,6-di-F | 154-158 | 397 | |
| 14 | CH₃ | CH₃ | Cl | 4-N≡C | 4-CH₃ | 166-170 | 382 | |
| 15 | CH₃ | CH₃ | F | 4-F | 4-CH₃, 6-Cl | | 393 | |
| 16 | CH₃ | Et | F | 4-F | 4-CH₃, 6-Cl | | 407 | |
| 17 | CH₃ | Et | F | 4-F | 6-Cl | 116-118 | 393 | |
| 18 | CH₃ | CH₃ | Cl | 4-N≡C | 4-F, 6-Cl | 182-186 | 420 | |
| 19 | CH₃ | CH₃ | Cl | 4-F | 4-F, 6-Cl | 153-157 | 413 | |
| 20 | CH₃ | CH₃ | Br | 4-F | 4-F | 127-131 | 424 | |
| 21 | CH₃ | CH₃ | Br | 4-F | 4-Br | 112-116 | 485 | |
| 22 | CH₃ | CH₃ | Cl | 6-F | 4-Cl | 173-177 | 396 | |
| 23 | CH₃ | CH₃ | Cl | 6-F | 4-CH₃O, 6-F | 167-171 | 409 | |
| 24 | CH₃ | CH₃ | Cl | 6-F | 4-CH₃, 6-Cl | 184-188 | 410 | |
| 25 | CH₃ | CH₃ | Cl | 6-F | 6-F | 130-134 | 379 | |
| 26 | CH₃ | CH₃ | Cl | q4-F | 4-CH₃ | 118-122 | 375 | |
| 27 | CH₃ | CH₃ | Cl | 4-CH₃O | 6-F | 145-149 | 391 | |
| 28 | CH₃ | CH₃ | Cl | 4-F | 4-CF₂HO | | 427 | |
| 29 | CH₃ | CH₃ | Cl | 4-F | 4-N≡CCH₂O | 189-193 | 416 | |
| 30 | CH₃ | CH₃ | Cl | 4-F | 5-F | | 379 | |
| 31 | CH₃ | CH₃ | Cl | 4-F | 4-CH₃, 6-Cl | 164-168 | 410 | |
| 32 | CH₃ | CH₃ | Cl | 4-F | 4,5-di-F | 123-127 | 397 | |
| 33 | CH₃ | CH₃ | Cl | 4-F | 4-Cl, 6-CH₃ | 155-159 | 410 | |
| 34 | CH₃ | CH₃ | Cl | 4-F | 3-Cl | 105-109 | 396 | |
| 35 | CH₃ | CH₃ | Me | 4-CH₃O | 6-Cl | 195-199 | 389 | |
| 36 | CH₃ | CH₃ | Cl | 4-F | 4-CF₂HCH₂O | | 442 | |
| 37 | CH₃ | CH₃ | Br | 4-F | 4,6-di-Cl | 185-189 | 475 | |
| 38 | CH₃ | CH₃ | Cl | 6-F | 6-Cl | 159-163 | 396 | |
| 39 | CH₃ | CH₃ | Cl | 6-F | 4,6-di-F | 146-150 | 397 | |
| 40 | CH₃ | CH₃ | Br | 4-F | 4-CH₃, 6-F | 152-156 | 438 | |
| 41 | CH₃ | CH₃ | Br | 4-F | 6-Cl | 161-165 | 440 | |
| 42 | CH₃ | CH₃ | Br | 4-F | 6-CH₃ | 148-152 | 420 | |
| 43 | CH₃ | CH₃ | Br | 4-F | 4-F, 6-Cl | 208-212 | 458 | |
| 44 | CH₃ | CH₃ | Cl | 6-F | 4-F | 138-142 | 379 | |
| 45 | CH₃ | Et | F | 4-F | 4-CH₃O | | 389 | |
| 46 | CH₃ | Et | F | 4-F | 6-F | | 377 | |
| 47 | CH₃ | CH₃ | Cl | 4-F | 4-I, 6-F | 143-148 | 505 | |
| 48 | CH₃ | CH₃ | Me | 4-CH₃O | 6-F | 145-150 | 371 | |
| 49 | CH₃ | CH₃ | Cl | 6-F | 4-F, 6-Cl | 174-178 | 414 | |
| 50 | CH₃ | Et | F | 4-F | 4,6-di-F | | | 393 |
| 51 | CH₃ | CH₃ | Br | 4-F | 4,6-di-F | 159-163 | 442 | |
| 52 | CH₃ | CH₃ | Br | 4-F | 4-CH₃O | | 435 | |
| 53 | CH₃ | CH₃ | Cl | 6-Cl | 6-F | 145-149 | 396 | |
| 54 | CH₃ | CH₃ | Cl | 6-Cl | 4-F | 151-155 | 396 | |
| 55 | CH₃ | CH₃ | F | 4-F | 4-Br, 6-Cl | 179-183 | 458 | |
| 56 | CH₃ | CH₃ | F | 4-F | 4-Br, 6-F | 136-140 | 442 | |
| 57 | CH₃ | CH₃ | F | 4-F | 4-CH₃O, 6-F | 143-147 | 393 | |
| 58 | CH₃ | CH₃ | F | 4-F | 4,6-di-F | 131-135 | 381 | |
| 59 | CH₃ | CH₃ | Cl | 4-F | 4,6-di-Cl | 180-184 | 429 | |
| 60 | CH₃ | CH₃ | Br | 4-F | 6-F | 132-136 | 423 | |
| 61 (Ex. 3) | CH₃ | CH₃ | Cl | 4-F | 4-Br, 6-F | 149-153 | 457 | |
| 62 | CH₃ | CH₃ | F | 4,6-di-F | 6-F | 141-145 | 381 | |
| 63 | CH₃ | CH₃ | F | 6-F | 6-F | 160-164 | 363 | |
| 64 | CH₃ | CH₃ | Cl | 4-CH₃O | 4,6-di-F | 169-174 | 409 | |
| 65 | CH₃ | CH₃ | Cl | 4-CH₃O | 4,6-di-Cl | 208-213 | 442 | |
| 66 | CH₃ | CH₃ | Cl | 4-CH₃O | 4-CH₃, 6-Cl | 164-169 | 422 | |
| 67 | CH₃ | CH₃ | F | 4-F | 4-F, 6-Cl | 163-167 | 397 | |
| 68 | CH₃ | CH₃ | Cl | 4,6-di-F | 6-F | 124-128 | 397 | |
| 69 | CH₃ | CH₃ | Cl | 4-Cl | 6-F | 169-173 | 395 | |
| 70 | CH₃ | CH₃ | F | 4-CH₃O, 6-F | 6-F | 148-152 | 393 | |
| 71 | Et | CH₃ | Cl | 4-F | 6-F | 112-116 | 393 | |
| 72 | CH₃ | Et | Cl | 4-F | 6-F | 117-121 | 394 | |
| 73 | CH₃ | CH₃ | Cl | 4-CH₃O | 4-F, 6-Cl | 167-172 | 426 | |
| 74 | CH₃ | CH₃ | Cl | 4-CH₃O | 4-Cl | 148-153 | 408 | |
| 75 | CH₃ | CH₃ | F | 6-F | 4,6-di-F | 195-199 | 381 | |
| 76 | CH₃ | CH₃ | F | 6-F | 4-F, 6-Cl | 184-188 | 397 | |
| 77 | CH₃ | CH₃ | F | 6-F | 6-Cl | 154-158 | 379 | |
| 78 | CH₃ | CH₃ | F | 4-F | 4-Cl | 178-182 | 379 | |
| 79 | CH₃ | CH₃ | F | 4-F | 6-Cl | 139-143 | 379 | |
| 80 | CH₃ | CH₃ | Cl | 4-F | 4-CH₃O, 6-Cl | 184-189 | 426 | |
| 81 | CH₃ | CH₃ | Cl | 4-CH₃O | 4-Cl, 6-F | 165-170 | 426 | |
| 82 | CH₃ | CH₃ | F | 4-CH₃O, 6-F | 4,6-di-Cl | 162-167 | 444 | |
| 83 | CH₃ | CH₃ | F | 4-CH₃O, 6-F | 4-F | 172-177 | 393 | |
| 84 | CH₃ | CH₃ | F | 4-CH₃O, 6-F | 4,6-di-F | 178-182 | 411 | |
| 85 | CH₃ | CH₃ | Cl | 4-F | 4-CH₃O, 6-F | 125-130 | 409 | |
| 86 | CH₃ | CH₃ | F | 4-CH₃O, 6-F | 4-Cl, 6-F | 144-149 | 427 | |
| 87 | CH₃ | CH₃ | F | 6-F | 4-CH₃O | | 375 | |
| 88 | CH₃ | CH₃ | F | 4-F | 4-CH₃O | | 375 | |
| 89 | CH₃ | CH₃ | Cl | 4,6-di-F | 4-CH₃O | | 409 | |
| 90 | CH₃ | CH₃ | Br | 4-F | 4-CH₃ | | 419 | |
| 91 | CH₃ | CH₃ | Cl | 4-F | 4-Br | 93-97 | 437 | |
| 92 | CH₃ | CH₃ | Cl | 4-F | 4-Cl | 98-102 | 394 | |
| 93 (Ex. 4) | CH₃ | CH₃ | Cl | 4-F | 4-CH₃, 6-F | 147-151 | 393 | |
| 94 | CH₃ | CH₃ | Cl | 4-F | 4-CF₃O | 124-128 | 445 | |
| 95 | CH₃ | Et | Cl | 4-F | 4,6-di-F | 107-111 | 412 | |
| 96 | CH₃ | CH₃ | F | 6-F | 4,6-di-Cl | 185-189 | 414 | |
| 97 | CH₃ | CH₃ | F | 6-F | 4-CH₃O, 6-F | 159-163 | 393 | |
| 98 | CH₃ | CH₃ | F | 6-F | 4-Cl, 6-F | 180-184 | 397 | |
| 99 | CH₃ | CH₃ | F | 6-F | 3-Cl | | 379 | |
| 100 | CH₃ | CH₃ | F | 6-F | 4-CH₃, 6-Cl | 100-104 | 393 | |
| 101 | CH₃ | CH₃ | F | 4-F | 4,6-di-Cl | 176-180 | 414 | |
| 102 | CH₃ | CH₃ | F | 4-F | 4-Cl, 6-F | 136-140 | 397 | |
| 103 | CH₃ | CH₃ | Cl | 4-F | 4-CH₃CH₂O | 95-99 | 405 | |
| 104 | CH₃ | CH₃ | Cl | 4-F | 4-i-PrO | 99-103 | 419 | |
| 105 | CH₃ | CH₃ | F | 6-F | 4-CH₃ | | 359 | |
| 106 | CH₃ | CH₃ | Cl | 4,6-di-F | 4-CH₃ | | 392 | |
| 107 | CH₃ | CH₃ | F | 4-F | 4-CH₃ | | 359 | |
| 108 | CH₃ | CH₃ | Cl | 4-F | 6-Cl | 145-149 | 395 | |
| 109 | CH₃ | CH₃ | Cl | 4-F | 4,6-di-F | 158-162 | 397 | |
| 110 | CH₃ | CH₃ | Cl | 4-F | 6-Br | 151-155 | 439 | |
| 111 | CH₃ | CH₃ | Cl | 4-F | 4-F | 120-124 | 379 | |
| 112 (Ex. 1) | CH₃ | CH₃ | Cl | 4-F | 6-F | 130-134 | 379 | |
| 113 (Exs. 2 & 5) | CH₃ | CH₃ | Cl | 4-N≡C | 6-F | 176-180 | 386 | |
| 114 | CH₃ | CH₃ | F | 4-N≡C, 6-F | 6-F | 185-189 | 388 | |
| 115 | CH₃ | CH₃ | Cl | 4-N≡C | 4-Cl, 6-F | 185-189 | 420 | |
| 116 | CH₃ | CH₃ | Cl | 4-N≡C | 4,6-di-Cl | 216-220 | 436 | |
| 117 | CH₃ | CH₃ | Cl | 4-F | 6-CH₃ | 154-158 | 375 | |
| 118 | CH₃ | CH₃ | F | 4-F | 6-F | 113-117 | 363 | |
| 119 | CH₃ | CH₃ | Cl | 4-F | 4-CH₃O | 79-83 | 391 | |
| 120 | CH₃ | CH₃ | Cl | 4-N≡C | 4,6-di-F | 191-195 | 404 | |
| 121 | CH₃ | CH₃ | Cl | 4-F | 4-Cl, 6-F | 131-135 | 413 | |
| 122 | CH₃ | C≡N | Cl | 4-F | 6-F | 166-170 | 390 | |
| 123 | CH₃ | CH₃ | Cl | 4-N≡C | 6-Cl | 196-199 | 402 | |
| 124 | CH₃ | CH₃ | Cl | 4-N≡C | 4-CH₃O | 137-139 | 398 | |
| 125 | CH₃ | CH₃ | Cl | 4-N≡C | 6-Br | 209-211 | 446 | |
| 126 | CH₃ | CH₃ | Cl | 4-F | — | | 361 | |
| 127 | CH₃ | CH₃ | Cl | 4-N≡C | 4-CH₃, 6-F | 166-170 | 400 | |
| 128 | CH₃ | Et | Cl | 4-F | 4-Br, 6-Cl | | 489 | |
| 129 | CH₃ | Et | Cl | 4-F | 6-Cl | | 423 | |
| 130 | CH₃ | CH₃ | Cl | 4-F | 5-Cl | | 395 | |
| 131 | CH₃ | CH₃ | Br | 4-F | 4-Cl, 6-F | 151-155 | | |
| 132 | CH₃ | CH₃ | Cl | 4-Cl | 4-Br, 6-F | 159-163 | | |
| 133 | CH₃ | CH₃ | Cl | 4-Cl | 6-Cl | 208-212 | | |
| 134 | CH₃ | CH₃ | F | 4-N≡C | — | 125-129 | | |
| 135 | CH₃ | CH₃ | F | 6-F | — | 149-153 | | |
| 136 | CH₃ | CH₃ | Cl | 6-F | — | 152-156 | | |
| 137 | CH₃ | Et | Cl | 4-F | 4-CH₃, 6-F | | 407 | |
| 138 | CH₃ | CH₃ | Cl | 4-Cl | 4-CH₃O, 6-F | 103-107 | | |
| 139 | CH₃ | CH₃ | Br | 4-F | — | 119-123 | | |
| 140 | CH₃ | CH₃ | Cl | 4-MeO | — | 105-109 | | |
| 141 | CH₃ | CH₃ | F | 4-F | 4-CH₃O, 6-Cl | 156-160 | | |
| 142 | CH₃ | CH₃ | Cl | 4-Cl | 4-CH₃O | | 407 | |
| 143 | CH₃ | CH₃ | Cl | 4-Cl | 4-CH₃, 6-F | 158-162 | | |
| 144 | CH₃ | CH₃ | Cl | 4-Cl | 6-CH₃ | 206-210 | | |
| 145 | CH₃ | CH₃ | Cl | 4-Cl | 4-CH₃, 6-Cl | | | |
| 146 | CH₃ | CH₃ | Cl | 4-Cl | 4,6-di-Cl | 180-184 | | |
| 147 | CH₃ | CH₃ | F | 4-CH₃O, 6-F | — | | 375 | |
| 148 | CH₃ | CH₃ | Cl | 4,6-di-F | — | | 379 | |
| 149 | CH₃ | CH₃ | F | 4,6-di-F | — | | 363 | |
| 150 | CH₃ | CH₃ | F | 4-N≡C, 6-F | 4-CH₃O | 186-190 | | |
| 151 | CH₃ | CH₃ | F | 4-Cl,6-F | 6-F | | 397 | |
| 152 | CH₃ | CH₃ | Cl | 4,6-di-F | 6-Cl | 152-156 | | |
| 153 | CH₃ | CH₃ | Cl | 4,6-di-F | 6-CH₃ | 141-145 | | |
| 154 | CH₃ | CH₃ | Br | 4,6-di-F | — | | 423 | |
| 155 | CH₃ | CH₃ | Cl | 4-N≡C | 4-Br, 6-F | 177-181 | | |
| 156 | CH₃ | CH₃ | Cl | 4-N≡C | 4-CH₃, 6-Cl | 230-234 | | |
| 157 | CH₃ | CH₃ | Cl | 4-N≡C | 6-CH₃ | 220-224 | | |
| 158 | CH₃ | CH₃ | F | 4,6-di-F | 6-Cl | 180-184 | | |
| 159 | CH₃ | CH₃ | F | 4,6-di-F | 4-CH₃O | | 392 | |
| 160 | CH₃ | CH₃ | F | 4-CH₃O, 6-F | 6-Cl | 168-172 | | |
| 161 | CH₃ | CH₃ | F | 4-CH₃O, 6-F | 4-CH₃O | | 405 | |
| 162 | CH₃ | CH₃ | F | 4-CH₃O, 6-F | 6-CH₃ | 154-158 | | |
| 163 | CH₃ | CH₃ | Br | 4,6-di-F | 6-Cl | 201-205 | | |
| 164 | CH₃ | CH₃ | Br | 4,6-di-F | 4-CH₃O | | 453 | |
| 165 | CH₃ | CH₃ | Br | 4-F | 4-CH₃O, 6-F | 177-181 | | |
| 166 | CH₃ | CH₃ | Cl | 4-N≡C | 4-CH₃O, 6-F | 185-189 | | |
| 167 | CH₃ | CH₃ | F | 4,6-di-F | 6-CH₃ | 151-155 | | |
| 168 | CH₃ | CH₃ | F | 4-Cl, 6-F | — | 118-122 | | |
| 169 | CH₃ | CH₃ | Br | 4,6-di-F | 6-CH₃ | 196-200 | | |
| 170 | CH₃ | Et | F | 4-Cl, 6-F | 6-F | 120-122 | | |
| 171 | CH₃ | Et | F | 4-F | 4-Br, 6-F | | 455 | |
| 173 | CH₃ | Et | Cl | 4-F | 4-CH₃O | | 405 | |
| 174 | CH₃ | Et | Cl | 4-F | 4-CH₃ | | | 388 |
| 175 | CH₃ | Et | F | 4-F | 4-CH₃ | | 373 | |
| 176 | CH₃ | CH₃ | F | 4-Cl | 4-F, 6-Cl | 184-188 | | |
| 177 | CH₃ | CH₃ | F | 4-F | 6-CH₃ | 130-134 | | |
| 178 | CH₃ | CH₃ | F | 4-F | 4-CH₃, 6-F | 108-112 | | |
| 179 | CH₃ | CH₃ | F | 4-N≡C | 4-CH₃O | 132-136 | | |
| 180 | CH₃ | CH₃ | Br | 4-N≡C | 4-CH₃O | 41-45 | | |
| 181 | CH₃ | CH₃ | Br | 4-F | 4-CH₃, 6-Cl | 174-178 | | |
| 182 | CH₃ | CH₃ | Cl | 4-CH₃O | 4-Br, 6-F | 180-184 | | |
| 183 | CH₃ | CH₃ | Cl | 4-CH₃O | 6-CH₃ | 208-212 | | |
| 184 | CH₃ | CH₃ | Cl | 4-CH₃O | 6-Cl | 189-193 | | |
| 185 | CH₃ | Et | F | 4-F | 4-CH₃, 6-F | | 391 | |
| 186 | CH₃ | CH₃ | Cl | 4-CH₃O | 4-CH₃O, 6-F | 156-160 | | |
| 187 | CH₃ | CH₃ | Cl | 4-CH₃O | 4-CH₃O | | 403 | |
| 188 | CH₃ | CH₃ | Cl | 4-CH₃O | 4-CH₃, 6-F | 135-139 | | |
| 189 | CH₃ | CH₃ | Cl | 4-Cl | — | | 377 | |
| 190 | CH₃ | CH₃ | F | 4-F | — | 132-136 | | |
| 191 | CH₃ | CH₃ | Br | 4-F | 4-Br, 6-F | 142-146 | | |
| 192 | CH₃ | CH₃ | Cl | 4-NO₂ | 6-F | 162-165 | | |
| 193 | CH₃ | CH₃ | Cl | 4,6-di-F | 4-CH₃, 6-F | 130-133 | | |
| 194 | CH₃ | CH₃ | F | 4,6-di-F | 4-CH₃, 6-F | 129-133 | | |
| 195 | CH₃ | CH₃ | Cl | 6-F | 4-CH₃ | 142-145 | | |
| 196 | CH₃ | CH₃ | Br | 4,6-di-F | 4-CH₃ | 117-120 | | |
| 197 | CH₃ | CH₃ | Cl | 6-F | 4-CH₃O | 133-136 | | |
| 198 | CH₃ | CH₃ | F | 4-N≡C | 4-CH₃, 6-F | 140-143 | | |
| 199 | CH₃ | CH₃ | Cl | 4,6-di-F | 4,6-di-F | | 415 | |
| 200 | CH₃ | CH₃ | F | 4-N≡C, 6-F | 4-CH₃, 6-F | 173-176 | | |
| 201 | CH₃ | CH₃ | Cl | 6-F | 4-CH₃, 6-F | 194-197 | | |

A dash “—” in the R⁵ column means that no R⁵ substituent is present and the remaining carbon valences are occupied by hydrogen atoms.

INDEX TABLE B

| Cmpd. No. | Structure | AP⁺ (M + 1) |
|---|---|---|
| 172 | 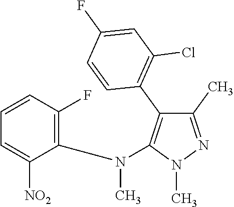 [SMILES: Cc1nn(C)c(N(C)c2c(F)cccc2[N+](=O)[O-])c1-c1ccc(F)cc1Cl] | 393 |

### Biological Examples of the Invention

General protocol for preparing test suspensions for Tests A-F: the test compounds were first dissolved in acetone in an amount equal to 3% of the final volume and then suspended at the desired concentration (in ppm) in acetone and purified water (50/50 mix by volume) containing 250 ppm of the surfactant PEG400 (polyhydric alcohol esters). The resulting test suspensions were then used in Tests A-F.

### Test A

The test solution was sprayed to the point of run-off on wheat seedlings. The following day the seedlings were inoculated with a spore suspension of Septoria tritici (the causal agent of wheat leaf blotch) and incubated in a saturated atmosphere at 24° C. for 48 h, and then moved to a growth chamber at 20° C. for 17 days, after which time disease ratings were made.

### Test B

The test solution was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time disease ratings were made.

### Test C

The test suspension was sprayed to the point of run-off on wheat seedlings. The following day the seedlings were inoculated with a spore dust of Blumeria graminis f. sp. tritici, (also known as Erysiphe graminis f. sp. tritici, the causal agent of wheat powdery mildew) and incubated in a growth chamber at 20° C. for 8 days, after which time visual disease ratings were made.

### Test D

The test solution was sprayed to the point of run-off on soybean seedlings. The following day the seedlings were inoculated with a spore suspension of Phakopsora pachyrhizi (the causal agent of Asian soybean rust) and incubated in a saturated atmosphere at 22° C. for 24 h and then moved to a growth chamber at 22° C. for 8 days, after which time visual disease ratings were made.

### Test E

The test suspension was sprayed to the point of run-off on tomato seedlings. The following day the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 days, after which time visual disease ratings were made.

### Test F

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 3 days, after which time visual disease ratings were made.

Results for Tests A-F are given in Table A below. A rating of 100 indicates 100% disease control and a rating of 0 indicates no disease control (relative to the controls). A dash (−) indicates the compound was not tested.

TABLE A

| Cmpd. | Rate | Test | Test | Test | Test | Test | Test |
|---|---|---|---|---|---|---|---|
| No. | in ppm | A | B | C | D | E | F |
| 1 | 50 | 100 | 100 | 100 | 84 | 100 | 99 |
| 2 | 50 | 100 | 100 | 99 | 94 | 99 | 61 |
| 3 | 50 | 100 | 89 | 56 | 0 | 70 | 0 |
| 4 | 50 | — | 86 | 76 | 0 | 74 | 0 |
| 5 | 50 | 97 | 86 | 76 | 19 | 95 | 0 |
| 6 | 50 | — | 74 | 27 | 0 | 55 | 0 |
| 7 | 50 | 100 | 99 | 94 | 96 | 99 | 89 |
| 8 | 50 | 99 | 99 | 92 | 73 | 99 | 73 |
| 9 | 50 | 100 | 100 | 97 | 0 | 97 | 99 |
| 10 | 50 | 100 | 99 | 48 | 0 | 68 | 9 |
| 11 | 50 | 100 | 98 | 97 | 0 | 98 | 99 |
| 12 | 50 | 100 | 99 | 81 | 0 | 97 | 53 |
| 13 | 50 | 100 | 99 | 97 | 19 | 68 | 89 |
| 14 | 50 | 100 | 97 | 83 | 48 | 96 | 83 |
| 15 | 50 | 100 | 100 | 98 | 61 | 99 | 53 |
| 16 | 50 | 100 | 99 | 98 | 50 | 99 | 0 |
| 17 | 50 | 100 | 92 | 94 | 0 | 99 | 9 |
| 18 | 50 | 100 | 100 | 89 | 92 | 100 | 99 |
| 19 | 50 | 100 | 100 | 100 | 98 | 100 | 99 |
| 20 | 50 | 100 | 89 | 92 | 0 | 99 | 26 |
| 21 | 50 | 100 | 86 | 84 | 0 | 95 | 17 |
| 22 | 50 | 100 | 93 | 97 | 0 | 99 | 64 |
| 23 | 50 | 100 | 100 | 100 | 99 | 97 | 99 |
| 24 | 50 | 100 | 89 | 0 | 0 | 97 | 0 |
| 25 | 50 | 100 | 100 | 99 | 98 | 100 | 98 |
| 26 | 50 | 100 | 99 | 94 | 77 | 100 | 68 |
| 27 | 50 | 100 | 100 | 91 | 87 | 100 | 99 |
| 28 | 50 | 91 | 68 | 69 | 0 | 99 | 0 |
| 29 | 50 | 97 | 98 | 87 | 81 | 99 | 26 |
| 30 | 50 | 100 | 86 | 91 | 0 | 67 | 0 |
| 31 | 50 | 100 | 100 | 100 | 98 | 99 | 76 |
| 32 | 50 | 93 | 85 | 56 | 98 | 99 | 0 |
| 33 | 50 | 100 | 100 | 99 | 92 | 99 | 85 |
| 34 | 50 | 100 | 54 | 13 | 0 | 71 | 0 |
| 35 | 50 | 99 | 85 | 0 | 0 | 0 | 0 |
| 36 | 50 | 100 | 89 | 56 | 25 | 100 | 0 |
| 37 | 50 | 100 | 100 | 98 | 0 | 88 | 78 |
| 38 | 50 | 100 | 99 | 95 | 0 | 88 | 0 |
| 39 | 50 | 100 | 100 | 99 | 73 | 88 | 99 |
| 40 | 50 | 100 | 100 | 100 | 82 | 98 | 100 |
| 41 | 50 | 100 | 100 | 94 | 77 | 77 | 0 |
| 42 | 50 | 100 | 100 | 96 | 0 | 91 | 0 |
| 43 | 50 | 100 | 100 | 97 | 65 | 96 | 64 |
| 44 | 50 | 100 | 99 | 98 | 0 | 95 | 46 |
| 45 | 50 | 100 | 86 | 95 | 0 | 99 | 33 |
| 46 | 50 | 100 | 98 | 99 | 0 | 99 | 81 |
| 47 | 50 | 100 | 97 | 89 | 92 | 95 | 96 |
| 48 | 50 | 100 | 95 | 89 | 0 | 97 | 97 |
| 49 | 50 | 100 | 100 | 95 | 25 | 94 | 26 |
| 50 | 50 | 100 | 98 | 100 | 0 | 92 | 98 |
| 51 | 50 | 100 | 100 | 100 | 79 | 98 | 99 |
| 52 | 50 | 100 | 100 | 90 | 59 | 99 | 94 |
| 53 | 50 | 100 | 100 | 98 | 0 | 98 | 98 |
| 54 | 50 | 100 | 91 | 93 | 0 | 21 | 0 |
| 55 | 50 | 100 | 99 | 90 | 0 | 34 | 0 |
| 56 | 50 | 100 | 100 | 100 | 65 | 99 | 99 |
| 57 | 50 | 100 | 100 | 100 | 96 | 100 | 100 |
| 58 | 50 | 100 | 100 | 99 | 36 | 99 | 99 |
| 59 | 50 | 100 | 100 | 99 | 96 | 99 | 92 |
| 60 | 50 | 100 | 100 | 100 | 97 | 99 | 99 |
| 60 | 10 | 99 | 100 | 85 | 0 | 99 | 75 |
| 61 | 50 | 100 | 100 | 99 | 84 | 99 | 99 |
| 62 | 50 | 100 | 100 | 100 | 100 | 99 | 99 |
| 63 | 50 | 100 | 99 | 86 | 0 | 100 | 96 |
| 64 | 50 | 100 | 100 | 98 | 97 | 98 | 99 |
| 65 | 50 | 94 | 89 | 56 | 66 | 16 | 46 |
| 66 | 50 | 100 | 100 | 98 | 97 | 98 | 96 |
| 67 | 50 | 100 | 100 | 89 | 69 | 98 | 33 |
| 68 | 50 | 100 | 100 | 100 | 99 | 100 | 99 |
| 68 | 10 | 100 | 100 | 96 | 32 | 100 | 98 |
| 69 | 50 | 100 | 100 | 97 | 77 | 100 | 99 |
| 70 | 50 | 100 | 100 | 99 | 84 | 99 | 99 |
| 71 | 50 | 100 | 100 | 99 | 82 | 99 | 98 |
| 72 | 50 | 100 | 100 | 99 | 31 | 98 | 99 |
| 72 | 10 | 100 | 85 | 81 | 12 | 98 | 63 |
| 73 | 50 | 100 | 100 | 96 | 98 | 99 | 97 |
| 74 | 50 | 99 | 86 | 81 | 74 | 99 | 85 |
| 75 | 50 | 100 | 99 | 90 | 0 | 57 | 98 |
| 76 | 50 | 100 | 99 | 95 | 25 | 87 | 0 |
| 77 | 50 | 100 | 100 | 95 | 73 | 72 | 9 |
| 78 | 50 | 100 | 89 | 95 | 0 | 68 | 0 |
| 79 | 50 | 100 | 100 | 91 | 44 | 98 | 0 |
| 80 | 50 | 100 | 100 | 97 | 50 | 95 | 98 |
| 81 | 50 | 100 | 99 | 89 | 0 | 95 | 99 |
| 82 | 50 | 100 | 100 | 58 | 59 | 96 | 97 |
| 83 | 50 | 99 | 91 | 99 | 0 | 98 | 99 |
| 84 | 50 | 100 | 100 | 99 | 48 | 90 | 99 |
| 85 | 50 | 100 | 100 | 97 | 96 | 94 | 98 |
| 86 | 50 | 100 | 100 | 98 | 77 | 96 | 99 |
| 87 | 50 | — | 99 | 98 | 0 | 98 | 93 |
| 88 | 50 | — | 97 | 84 | 0 | 99 | 9 |
| 89 | 50 | — | 100 | 99 | 80 | 99 | 99 |
| 90 | 50 | 100 | 95 | 87 | 0 | 99 | 9 |
| 91 | 50 | 99 | 88 | 84 | 0 | 99 | 26 |
| 92 | 50 | 100 | 96 | 89 | 0 | 100 | 79 |
| 93 | 50 | 100 | 100 | 99 | 100 | 100 | 99 |
| 93 | 10 | 99 | 100 | 97 | 87 | 99 | 99 |
| 94 | 50 | 73 | 0 | 72 | 73 | 0 | 9 |
| 95 | 50 | 100 | 100 | 100 | 84 | 99 | 99 |
| 96 | 50 | 100 | 95 | 0 | 0 | 80 | 0 |
| 97 | 50 | 100 | 100 | 100 | 96 | 100 | 99 |
| 98 | 50 | 100 | 97 | 93 | 0 | 99 | 98 |
| 99 | 50 | 99 | 0 | 0 | 0 | 24 | 0 |
| 100 | 50 | 100 | 89 | 21 | 0 | 26 | 0 |
| 101 | 50 | 100 | 99 | 89 | 44 | 99 | 0 |
| 102 | 50 | 100 | 100 | 100 | 48 | 99 | 99 |
| 103 | 50 | 97 | 80 | 50 | 0 | 83 | 0 |
| 104 | 50 | 60 | 0 | 72 | 0 | 0 | 0 |
| 105 | 50 | 99 | 74 | 86 | 0 | 73 | 0 |
| 106 | 50 | — | 100 | 98 | 0 | 99 | 68 |
| 107 | 50 | — | 91 | 61 | 0 | 98 | 0 |
| 108 | 50 | 100 | 100 | 97 | 86 | 100 | 77 |
| 109 | 50 | 100 | 100 | 100 | 93 | 100 | 99 |
| 110 | 50 | 100 | 98 | 56 | 73 | 99 | 0 |
| 111 | 50 | 100 | 99 | 99 | 0 | 100 | 93 |
| 112 | 50 | 88 | 100 | 100 | 99 | 100 | 100 |
| 112 | 10 | 72 | 100 | 98 | 52 | 100 | 98 |
| 113 | 50 | 100 | 99 | 97 | 13 | 99 | 99 |
| 114 | 50 | 100 | 98 | 99 | 73 | 100 | 99 |
| 115 | 50 | 100 | 100 | 94 | 77 | 99 | 99 |
| 116 | 50 | 99 | 99 | 56 | 0 | 63 | 82 |
| 117 | 50 | 99 | 100 | 95 | 0 | 99 | 76 |
| 118 | 50 | 100 | 100 | 98 | 0 | 100 | 99 |
| 118 | 10 | 100 | 80 | 27 | 0 | 99 | 60 |
| 119 | 50 | 100 | 100 | 99 | 92 | 100 | 99 |
| 120 | 50 | 100 | 100 | 89 | 44 | 100 | 99 |
| 121 | 50 | 100 | 100 | 100 | 99 | 100 | 100 |
| 122 | 50 | 100 | 100 | 94 | 0 | 100 | 99 |
| 123 | 50 | 100 | 97 | 81 | 0 | 100 | 66 |
| 124 | 50 | 100 | 100 | 99 | 25 | 100 | 99 |
| 125 | 50 | 100 | 96 | 0 | 0 | 99 | 9 |
| 126 | 50 | 98 | 85 | 96 | 0 | 100 | 68 |
| 127 | 50 | 100 | 99 | 92 | 96 | 100 | 100 |
| 128 | 50 | 100 | 98 | 96 | 31 | 99 | 33 |
| 129 | 50 | 100 | 100 | 97 | 84 | 99 | 88 |
| 130 | 50 | 85 | 86 | 20 | 0 | 85 | 0 |
| 131 | 50 | 100 | 100 | 99 | 80 | 99 | 100 |
| 132 | 50 | 100 | 98 | 47 | 0 | 99 | 96 |
| 133 | 50 | 100 | 86 | 0 | 0 | 97 | 0 |
| 134 | 50 | 100 | 23 | 71 | 0 | 70 | 0 |
| 135 | 50 | 100 | 68 | 93 | 0 | 99 | 0 |
| 136 | 50 | 100 | 80 | 88 | 0 | 99 | 0 |
| 137 | 50 | 100 | 100 | 100 | 91 | 99 | 99 |
| 138 | 50 | 100 | 100 | 93 | 67 | 99 | 100 |
| 139 | 50 | 98 | 86 | 76 | 0 | 100 | 33 |
| 140 | 50 | 99 | 86 | 31 | 0 | 100 | 93 |
| 141 | 50 | 99 | 100 | 99 | 0 | 99 | 83 |
| 142 | 50 | 100 | 91 | 39 | 25 | 99 | 80 |
| 143 | 50 | 99 | 100 | 96 | 87 | 99 | 100 |
| 144 | 50 | 100 | 68 | 35 | 69 | 0 | 0 |
| 145 | 50 | 98 | 92 | 52 | 0 | 85 | 24 |
| 146 | 50 | 97 | 98 | 0 | 0 | 0 | 40 |
| 147 | 50 | 96 | 86 | 97 | 0 | 100 | 99 |
| 148 | 50 | 100 | 99 | 98 | 13 | 99 | 100 |
| 149 | 50 | 100 | 80 | 97 | 0 | 99 | 96 |
| 150 | 50 | 100 | 100 | 98 | 13 | 100 | 99 |
| 151 | 50 | 100 | 100 | 99 | 0 | 99 | 99 |
| 152 | 50 | 99 | 100 | 98 | 91 | 99 | 92 |
| 153 | 50 | 99 | 100 | 99 | 73 | 99 | 72 |
| 154 | 50 | 99 | 80 | 98 | 0 | 99 | 51 |
| 155 | 50 | 99 | 99 | 90 | 0 | 100 | 93 |
| 156 | 50 | 91 | 91 | 66 | 0 | 0 | 0 |
| 157 | 50 | 100 | 97 | 76 | 0 | 9 | 0 |
| 158 | 50 | 99 | 100 | 99 | 87 | 99 | 83 |
| 159 | 50 | 100 | 100 | 100 | 13 | 99 | 99 |
| 160 | 50 | 98 | 100 | 96 | 86 | 100 | 99 |
| 161 | 50 | 100 | 100 | 100 | 0 | 100 | 99 |
| 162 | 50 | 100 | 100 | 99 | 77 | 97 | 98 |
| 163 | 50 | 99 | 100 | 98 | 78 | 88 | 76 |
| 164 | 50 | 100 | 100 | 98 | 0 | 100 | 99 |
| 165 | 50 | 99 | 100 | 98 | 94 | 100 | 100 |
| 166 | 50 | 99 | 100 | 98 | 98 | 99 | 100 |
| 167 | 50 | 100 | 100 | 98 | 65 | 95 | 98 |
| 168 | 50 | 100 | 74 | 97 | 0 | 99 | 40 |
| 169 | 50 | 100 | 100 | 100 | 0 | 98 | 0 |
| 170 | 50 | 100 | 99 | 99 | 0 | 100 | 99 |
| 171 | 50 | 100 | 91 | 99 | 0 | 97 | 88 |
| 172 | 50 | 100 | 100 | 100 | 0 | 99 | 40 |
| 173 | 50 | 100 | 100 | 98 | 0 | 98 | 93 |
| 174 | 50 | 100 | 95 | 92 | 0 | 99 | 39 |
| 175 | 50 | 100 | 86 | 79 | 0 | 88 | 0 |
| 176 | 50 | 100 | 97 | 0 | 0 | 9 | 0 |
| 177 | 50 | 100 | 97 | 50 | 0 | 100 | 9 |
| 178 | 50 | 100 | 100 | 99 | 97 | 100 | 100 |
| 179 | 50 | 100 | 96 | 93 | 0 | 99 | 85 |
| 180 | 50 | 100 | 98 | 79 | 0 | 99 | 98 |
| 181 | 50 | 100 | 100 | 99 | 78 | 99 | 99 |
| 182 | 50 | 100 | 99 | 76 | 0 | 100 | 99 |
| 183 | 50 | 100 | 99 | 78 | 0 | 100 | 58 |
| 184 | 50 | 100 | 100 | 21 | 0 | 100 | 26 |
| 185 | 50 | 100 | 100 | 99 | 0 | 96 | 99 |
| 186 | 50 | 100 | 100 | 99 | 94 | 100 | 100 |
| 187 | 50 | 99 | 100 | 91 | 0 | 100 | 100 |
| 188 | 50 | 100 | 100 | 94 | 84 | 100 | 100 |
| 189 | 50 | 99 | 68 | 57 | 0 | 99 | 0 |
| 190 | 50 | 100 | 32 | 91 | 0 | 100 | 9 |
| 191 | 50 | 100 | 100 | 99 | 13 | 100 | 100 |
| 192 | 50 | 100 | 99 | 76 | 19 | 99 | 93 |
| 193 | 50 | 100 | 100 | 100 | 99 | 99 | 100 |
| 194 | 50 | 100 | 100 | 100 | 99 | 99 | 100 |
| 195 | 50 | 100 | 98 | 97 | 0 | 98 | 73 |
| 196 | 50 | 100 | 99 | 98 | 0 | 98 | 91 |
| 197 | 50 | 100 | 99 | 93 | 0 | 98 | 98 |
| 198 | 50 | 99 | 99 | 95 | 89 | 99 | 100 |
| 199 | 50 | 99 | 100 | 100 | 100 | 99 | 100 |
| 200 | 50 | 99 | 100 | 99 | 100 | 98 | 100 |
| 201 | 50 | 100 | 100 | 99 | 98 | 99 | 99 |

The test results presented above in Table A for compounds of Formula 1 illustrate the fungicidal activity of component (a) contributing to the plant disease control utility of compositions comprising component (a) in combination with component (b) and optionally at least one additional fungicidal compound according to the present invention.

### Biological Comparative Examples

General protocol for preparing test suspensions for Tests A1-F1: the test compounds were first dissolved in acetone in an amount equal to 3% of the final volume and then suspended at the desired concentration (in ppm) in acetone and purified water (50/50 mix by volume) containing 250 ppm of the surfactant PEG400 (polyhydric alcohol esters). The resulting test suspensions were then used in Tests A1-F1.

### Test A1

The test solution was sprayed to the point of run-off on wheat seedlings. The following day the seedlings were inoculated with a spore suspension of Septoria tritici (the causal agent of wheat leaf blotch) and incubated in a saturated atmosphere at 24° C. for 48 h, and then moved to a growth chamber at 20° C. for 17 days, after which time disease ratings were made.

### Test B1

The test solution was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time disease ratings were made.

### Test C1

The test suspension was sprayed to the point of run-off on wheat seedlings. The following day the seedlings were inoculated with a spore dust of Blumeria graminis f. sp. tritici, (also known as Erysiphe graminis f. sp. tritici, the causal agent of wheat powdery mildew) and incubated in a growth chamber at 20° C. for 8 days, after which time visual disease ratings were made.

### Test E1

The test suspension was sprayed to the point of run-off on tomato seedlings. The following day the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 days, after which time visual disease ratings were made.

### Test F1

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 3 days, after which time visual disease ratings were made.

Results for Tests A1-F1 are given in Table B below. A rating of 100 indicates 100% disease control and a rating of 0 indicates no disease control (relative to the controls). The data is presented for the following compounds:

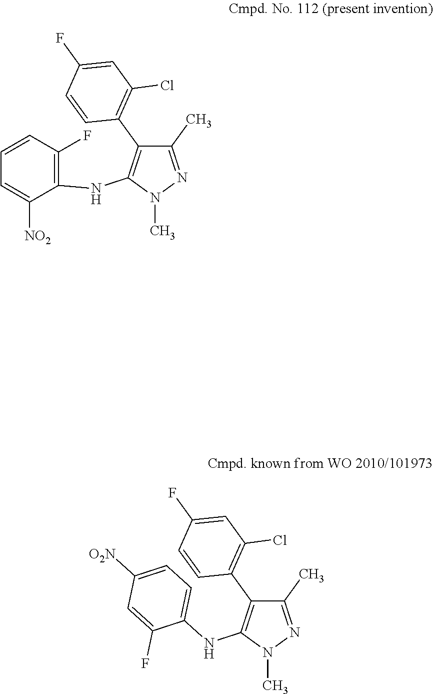 [SMILES: Cc1nn(C)c(Nc2c(F)cccc2[N+](=O)[O-])c1-c1ccc(F)cc1Cl ; Cc1nn(C)c(Nc2ccc([N+](=O)[O-])cc2F)c1-c1ccc(F)cc1Cl]

TABLE B

| | Rate | Test | Test | Test | Test | Test |
|---|---|---|---|---|---|---|
| | in ppm | A1 | B1 | C1 | E1 | F1 |
| Cmpd. No. 112 | 50 | 100 | 100 | 98 | 99 | 100 |
| | 10 | 81 | 86 | 97 | 99 | 99 |
| Cmpd. from | 50 | 91 | 0 | 0 | 40 | 0 |
| WO 2010/101973 | 10 | 45 | 0 | 0 | 0 | 0 |

What is claimed is:

1. 1. A fungicidal composition comprising:
(a) at least one compound selected from the compounds of Formula 1, N-oxides, and salts thereof: 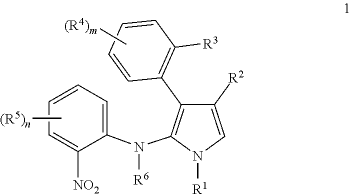
wherein
R¹ is C₁-C₂ alkyl;
R² is cyano, halogen, C₁-C₂ alkyl or C₁-C₂ haloalkyl;
R³ is halogen or methyl;
each R⁴ is independently halogen, cyano, nitro, C₁-C₃ alkyl, C₁-C₃ alkoxy, C₁-C₃ haloalkoxy, C₂-C₆ alkenyloxy, C₂-C₆ alkynyloxy, C₂-C₆ cyanoalkoxy, C₂-C₆ alkoxyalkyl or C₂-C₆ alkoxyalkoxy;
each R⁵ is independently halogen, C₁-C₃ alkyl, C₂-C₆ alkoxyalkyl, C₁-C₃ alkoxy, C₁-C₃ haloalkoxy, C₂-C₆ alkenyloxy, C₂-C₆ alkynyloxy, C₂-C₆ cyanoalkoxy or C₂-C₆ alkoxyalkoxy;
m and n are each independently 0, 1, 2 or 3;
R⁶ is H; or C₁-C₃ alkyl or C₁-C₃ haloalkyl, each optionally substituted with up to 2 substituents independently selected from R^{6a}; or amino, C₂-C₄ alkenyl, C₂-C₄ alkynyl, C₃-C₆ cycloalkyl, CH(═O), S(═O)₂OM, S(═O)ᵤR⁷, (C═W)R⁸ or OR⁹;
each R^{6a} is independently cyano, C₃-C₆ cycloalkyl, C₁-C₃ alkoxy, C₁-C₃ haloalkoxy, C₁-C₃ alkylthio, C₁-C₃ alkylsulfinyl or C₁-C₃ alkylsulfonyl;
M is K or Na;
u is 0, 1 or 2;
R⁷ is C₁-C₃ alkyl or C₁-C₃ haloalkyl;
W is O or S;
R⁸ is C₁-C₃ alkyl, C₂-C₄ alkoxyalkyl, C₂-C₄ alkylaminoalkyl, C₃-C₆ dialkylaminoalkyl, C₁-C₃ alkoxy, C₁-C₃ alkylthio or C₂-C₄ alkylthioalkyl;
R⁹ is H; or C₁-C₃ alkyl or C₁-C₃ haloalkyl, each optionally substituted with up to 2 substituents independently selected from R^{9a}; or CH(═O), C₃-C₆ cycloalkyl, S(═O)₂OM or (C═W)R¹⁰;
each R^{9a} is independently cyano, C₃-C₆ cycloalkyl, C₁-C₃ alkoxy, C₁-C₃ haloalkoxy, C₁-C₃ alkylthio, C₁-C₃ alkylsulfinyl or C₁-C₃ alkylsulfonyl; and
R¹⁰ is C₁-C₃ alkyl, C₂-C₄ alkoxyalkyl, C₂-C₄ alkylaminoalkyl, C₃-C₆ dialkylaminoalkyl, C₁-C₃ alkoxy, C₁-C₃ alkylthio or C₂-C₄ alkylthioalkyl; and
(b) at least one additional fungicidal compound;
provided that the compound of Formula 1 is not:
4-(2,6-difluoro-4-methoxyphenyl)-N-(2,4-difluoro-6-nitrophenyl)-1,3-dimethyl-1H-pyrazol-5-amine;
4-(2-chloro-4-fluorophenyl)-1,3-dimethyl-N-(2-nitrophenyl)-1H-pyrazol-5-amine;
4-(2-chloro-4-fluorophenyl)-N-(2,4-difluoro-6-nitrophenyl)-1,3-dimethyl-1H-pyrazol-5-amine;
4-(2-chloro-4-fluorophenyl)-3-ethyl-1-methyl-N-(2-nitrophenyl)-1H-pyrazol-5-amine;
4-(2-chloro-4-fluorophenyl)-1-methyl-N-(2-nitrophenyl)-3-(trifluoromethyl)-1H-pyrazol-5-amine;
4-(2,6-difluoro-4-methoxyphenyl)-N-(2-methoxy-6-nitrophenyl)-1,3-dimethyl-1H-pyrazol-5-amine;
4-(2-chloro-4-fluorophenyl)-N-(2-methoxy-6-nitrophenyl)-1,3-dimethyl-1H-pyrazol-5-amine;
N-(2-chloro-6-nitrophenyl)-4-(2,6-difluoro-4-methoxyphenyl)-1,3-dimethyl-1H-pyrazol-5-amine;
N-(2-chloro-3-fluoro-6-nitrophenyl)-4-(2,6-difluoro-4-methoxyphenyl)-1,3-dimethyl-1H-pyrazol-5-amine;
4-(2-chloro-4-fluorophenyl)-1,3-dimethyl-N-(2-methyl-6-nitrophenyl)-1H-pyrazol-5-amine;
N-(2-bromo-4-fluoro-6-nitrophenyl)-4-(2-chloro-4-fluorophenyl)-1,3-dimethyl-1H-pyrazol-5-amine;
4-(2-chloro-4-fluorophenyl)-N-(4-methoxy-2-nitrophenyl)-1,3-dimethyl-1H-pyrazol-5-amine;
4-(2,6-difluoro-4-methoxyphenyl)-N-(4-fluoro-2-nitrophenyl)-1,3-dimethyl-1H-pyrazol-5-amine;
4-(2,6-difluoro-4-methoxyphenyl)-N-(4-methoxy-2-nitrophenyl)-1,3-dimethyl-1H-pyrazol-5-amine;
N-(4-chloro-2-nitrophenyl)-4-(2,6-difluoro-4-methoxyphenyl)-1,3-dimethyl-1H-pyrazol-5-amine;
4-(2,6-difluoro-4-methoxyphenyl)-1,3-dimethyl-N-[2-nitro-4-(2-propyn-1-yloxy) phenyl]-1H-pyrazol-5-amine;
4-(2,6-difluoro-4-methoxyphenyl)-1,3-dimethyl-N-[2-nitro-4-(2-propen-1-yloxy) phenyl]-1H-pyrazol-5-amine;
N-(4-bromo-2-nitrophenyl)-4-(2,6-difluoro-4-methoxyphenyl)-1,3-dimethyl-1H-pyrazol-5-amine;
N-(4-chloro-2-fluoro-6-nitrophenyl)-4-(2,6-difluoro-4-methoxyphenyl)-1,3-dimethyl-1H-pyrazol-5-amine;
3-chloro-4-(2-chloro-4-fluorophenyl)-N-(2,4-difluoro-6-nitrophenyl)-1-methyl-1H-pyrazol-5-amine;
4-(2,6-difluoro-4-methoxyphenyl)-1,3-dimethyl-N-[4-methyl-2-nitrophenyl]-1H-pyrazol-5-amine;
4-(2-chloro-4-fluorophenyl)-1,3-dimethyl-N-(4-methyl-2-nitrophenyl)-1H-pyrazol-5-amine; and
N-(4-bromo-2-fluoro-6-nitrophenyl)-4-(2,6-difluoro-4-methoxyphenyl)-1,3-dimethyl-1H-pyrazol-5-amine.

2. The composition of claim 1 wherein component (a) comprises a compound of Formula 1 or salt thereof, wherein
R¹ is methyl;
R² is cyano, halogen or C₁-C₂ alkyl;
R³ is halogen;
each R⁴ is independently halogen, cyano, methyl, methoxy, halomethoxy, C₂-C₄ alkenyloxy, C₂-C₄ alkynyloxy or C₂-C₄ cyanoalkoxy;
each R⁵ is independently halogen, methyl, methoxy, halomethoxy, C₂-C₄ alkenyloxy, C₂-C₄ alkynyloxy or C₂-C₄ cyanoalkoxy;
R⁶ is H; or C₁-C₂ alkyl or C₁-C₂ haloalkyl, each optionally substituted with up to 1 substituent selected from R^{6a}; or S(═O)ᵤR⁷ or OR⁹;
R^{6a} is cyano, C₃-C₆ cycloalkyl or C₁-C₃ alkoxy;
R⁷ is methyl or halomethyl;
R⁹ is H; or C₁-C₂ alkyl or C₁-C₂ haloalkyl, each optionally substituted with up to 1 substituent selected from R^{9a}; and
R^{9a} is cyano, C₃-C₆ cycloalkyl or C₁-C₃ alkoxy.

3. The composition of claim 2 wherein component (a) comprises a compound of Formula 1 or salt thereof, wherein
R² is methyl or ethyl;
R³ is Br, Cl or F;
each R⁴ is independently halogen, cyano, methyl or methoxy;
m is 1 and R⁴ is at the para position; or m is 1 and R⁴ is at the ortho position; or m is 2 and one R⁴ is at the para position, and the other is at the ortho position;
each R⁵ is independently halogen, methyl or methoxy;
n is 1 and R⁵ is at the para position; or n is 1 and R⁵ is at the ortho position; or n is 2 and one R⁵ is at the para position, and the other is at the ortho position; and
R⁶ is H or methyl.

4. The composition of claim 3 wherein component (a) comprises a compound of Formula 1 or salt thereof, wherein
R² is methyl;
each R⁴ is independently Br, Cl, F, cyano or methoxy;
each R⁵ is independently Br, Cl, F, methyl or methoxy; and
R⁶ is H.

5. The composition of claim 4 wherein component (a) comprises a compound of Formula 1 or salt thereof, wherein
each R⁴ is independently Br, Cl or F; and
m and n are each 1 and R⁴ is at the para position and R⁵ is at the ortho position; or m is 1 and R⁴ is at the para position, and n is 2 and one R⁵ is at the para position and the other is at the ortho position; or m is 2 and one R⁴ is at the para position and the other is at the ortho position, and n is 1 and R⁵ is at the ortho position.

6. The composition of claim 5 wherein component (a) comprises a compound of Formula 1 or salt thereof, wherein
R⁴ is Cl or F;
each R⁵ is independently Cl, F or methyl; and
m and n are each 1 and R⁴ is at the para position and R⁵ is at the ortho position; or m is 1 and R⁴ is at the para position, and n is 2 and one R⁵ is at the para position and the other is at the ortho position.

7. The composition of claim 1 wherein component (a) comprises a compound of Formula 1 or salt thereof, wherein
R¹ is methyl;
R² is methyl or ethyl;
R³ is halogen;
each R⁴ is independently Br, Cl, F, cyano or methoxy;
m is 1 and R⁴ is at the para position; or m is 1 and R⁴ is at the ortho position; or m is 2 and one R⁴ is at the para position, and the other is at the ortho position;
n is 0; and
R⁶ is H or methyl.

8. The composition of claim 7 wherein component (a) comprises a compound of Formula 1 or salt thereof, wherein
R² is methyl;
R³ is Br, Cl or F;
each R⁴ is independently Br, Cl or F;
m is 1 and R⁴ is at the para position; or m is 1 and R⁴ is at the ortho position; or m is 2 and one R⁴ is at the para position, and the other is at the ortho position; and
R⁶ is H.

9. The composition of claim 1 wherein component (a) comprises a compound selected from the group consisting of
4-(2-bromo-4,6-difluorophenyl)-N-(2-fluoro-6-nitrophenyl)-1,3-dimethyl-1H-pyrazol-5-amine,
3-chloro-4-[5-[(2-chloro-4-fluoro-6-nitrophenyl)amino]-1,3-dimethyl-1H-pyrazol-4-yl]benzonitrile,
N-(2-chloro-4-fluoro-6-nitrophenyl)-4-(2-chloro-4-fluorophenyl)-1,3-dimethyl-1H-pyrazol-5-amine,
4-(2-chloro-6-fluorophenyl)-N-(2-fluoro-4-methoxy-6-nitrophenyl)-1,3-dimethyl-1H-pyrazol-5-amine,
4-(2,4-difluorophenyl)-N-(2-fluoro-4-methoxy-6-nitrophenyl)-1,3-dimethyl-1H-pyrazol-5-amine,
4-(2-bromo-4-fluorophenyl)-N-(2-fluoro-6-nitrophenyl)-1,3-dimethyl-1H-pyrazol-5-amine,
4-(2-chloro-4,6-difluorophenyl)-N-(2-fluoro-6-nitrophenyl)-1,3-dimethyl-1H-pyrazol-5-amine,
4-(2-chloro-4-fluorophenyl)-N-(2-fluoro-6-nitrophenyl)-3-ethyl-1-methyl-1H-pyrazol-5-amine,
N-(2-chloro-4-fluoro-6-nitrophenyl)-4-(2-chloro-4-methoxyphenyl)-1,3-dimethyl-1H-pyrazol-5-amine,
4-(2-chloro-4-fluorophenyl)-N-(2-fluoro-4-methyl-6-nitrophenyl)-1,3-dimethyl-1H-pyrazol-5-amine,
4-(2-chloro-4-fluorophenyl)-N-(4-fluoro-2-nitrophenyl)-1,3-dimethyl-1H-pyrazol-5-amine,
4-(2-chloro-4-fluorophenyl)-N-(2-fluoro-6-nitrophenyl)-1,3-dimethyl-1H-pyrazol-5-amine,
4-(2,4-difluorophenyl)-N-(2-fluoro-6-nitrophenyl)-1,3-dimethyl-1H-pyrazol-5-amine,
N-(4-chloro-2-fluoro-6-nitrophenyl)-4-(2-chloro-4-fluorophenyl)-1,3-dimethyl-1H-pyrazol-5-amine and
3-chloro-4-1-[5-[(2-fluoro-4-methyl-6-nitrophenyl)amino]-1,3-dimethyl-1H-pyrazol-4-yl]benzonitrile.

10. The composition of claim 8 wherein component (a) comprises a compound selected from the group consisting of
4-(2-bromo-4-fluorophenyl)-N-(2-fluoro-6-nitrophenyl)-1,3-dimethyl-1H-pyrazol-5-amine,
4-(2-chloro-4,6-difluorophenyl)-N-(2-fluoro-6-nitrophenyl)-1,3-dimethyl-1H-pyrazol-5-amine,
4-(2-chloro-4-fluorophenyl)-N-(2-fluoro-6-nitrophenyl)-3-ethyl-1-methyl-1H-pyrazol-5-amine,
4-(2-chloro-4-fluorophenyl)-N-(2-fluoro-4-methyl-6-nitrophenyl)-1,3-dimethyl-1H-pyrazol-5-amine,
4-(2-chloro-4-fluorophenyl)-N-(2-fluoro-6-nitrophenyl)-1,3-dimethyl-1H-pyrazol-5-amine and
4-(2,4-difluorophenyl)-N-(2-fluoro-6-nitrophenyl)-1,3-dimethyl-1H-pyrazol-5-amine.

11. The composition of any one of claims 1 through 7 wherein component (b) includes at least one fungicidal compound selected from the group consisting of:
(b1) methyl benzimidazole carbamate (MBC) fungicides;
(b2) dicarboximide fungicides;
(b3) demethylation inhibitor (DMI) fungicides;
(b4) phenylamide (PA) fungicides;
(b5) amine/morpholine fungicides;
(b6) phospholipid biosynthesis inhibitor fungicides;
(b7) succinate dehydrogenase inhibitor (SDHI) fungicides;
(b8) hydroxy(2-amino-)pyrimidine fungicides;
(b9) anilinopyrimidine (AP) fungicides;
(b10)N-phenyl carbamate fungicides;
(b11) quinone outside inhibitor (QoI) fungicides;
(b12) phenylpyrrole (PP) fungicides;
(b13) azanaphthalene fungicides;
(b14) cell peroxidation inhibitor fungicides;
(b15) melanin biosynthesis inhibitor-reductase (MBI-R) fungicides;
(b16a) melanin biosynthesis inhibitor-dehydratase (MBI-D) fungicides;
(b16b) melanin biosynthesis inhibitor-polyketide synthase (MBI-P) fungicides;
(b17) keto reductase inhibitor (KRI) fungicides;
(b18) squalene-epoxidase inhibitor fungicides;
(b19) polyoxin fungicides;
(b20) phenylurea fungicides;
(b21) quinone inside inhibitor (QiI) fungicides;
(b22) benzamide and thiazole carboxamide fungicides;
(b23) enopyranuronic acid antibiotic fungicides;
(b24) hexopyranosyl antibiotic fungicides;
(b25) glucopyranosyl antibiotic: protein synthesis fungicides;
(b26) glucopyranosyl antibiotic fungicides;
(b27) cyanoacetamideoxime fungicides;
(b28) carbamate fungicides;
(b29) oxidative phosphorylation uncoupling fungicides;
(b30) organo tin fungicides;
(b31) carboxylic acid fungicides;
(b32) heteroaromatic fungicides;
(b33) phosphonate fungicides;
(b34) phthalamic acid fungicides;
(b35) benzotriazine fungicides;
(b36) benzene-sulfonamide fungicides;
(b37) pyridazinone fungicides;
(b38) thiophene-carboxamide fungicides;
(b39) complex I NADH oxido-reductase inhibitor fungicides;
(b40) carboxylic acid amide (CAA) fungicides;
(b41) tetracycline antibiotic fungicides;
(b42) thiocarbamate fungicides;
(b43) benzamide fungicides;
(b44) microbial fungicides;
(b45) quinone outside inhibitor, stigmatellin binding (QoSI) fungicides;
(b46) plant extract fungicides;
(b47) cyanoacrylate fungicides;
(b48) polyene fungicides;
(b49) oxysterol binding protein inhibitor (OSBPI) fungicides;
(b50) aryl-phenyl-ketone fungicides;
(b51) host plant defense induction fungicides;
(b52) multi-site activity fungicides;
(b53) biologicals with multiple modes of action;
(b54) fungicides other than fungicides of component (a) and components (b1) through (b53); and salts of compounds of (b1) through (b54).

12. The composition of claim 11 wherein component (b) comprises at least one fungicidal compound from each of two different groups selected from (b1) through (b54).

13. The composition of any one of claims 1 through 7 wherein component (b) includes at least one compound selected from acibenzolar-S-methyl, aldimorph, ametoctradin, amisulbrom, anilazine, azaconazole, azoxystrobin, benalaxyl, benalaxyl-M, benodanil, benomyl, benthiavalicarb, benthiavalicarb-isopropyl, benzovindiflupyr, bethoxazin, binapacryl, biphenyl, bitertanol, bixafen, blasticidin-S, boscalid, bromuconazole, bupirimate, carboxin, carpropamid, captafol, captan, carbendazim, chloroneb, chlorothalonil, chlozolinate, clotrimazole, copper hydroxide, copper salts, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole, diniconazole-M, dinocap, dithianon, dodemorph, dodine, edifenphos, enestroburin, epoxiconazole, ethaboxam, ethirimol, etridiazole, famoxadone, fenamidone, fenarimol, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, fluazinam, fludioxonil, fluindapyr, flumetover, flumorph, fluopicolide, fluopyram, fluoroimide, fluoxastrobin, fluquinconazole, flusilazole, flusulfamide, flutianil, flutolanil, flutriafol, fluxapyroxad, folpet, fosetyl-aluminum, fuberidazole, furalaxyl, furametpyr, hexaconazole, hymexazol, guazatine, imazalil, imibenconazole, iminoctadine, iodocarb, ipconazole, ipfentrifluconazole, iprobenfos, iprodione, iprovalicarb, isoprothiolane, isopyrazam, isotianil, kasugamycin, kresoxim-methyl, mancozeb, mandipropamid, maneb, mepronil, meptyldinocap, metalaxyl, metalaxyl-M, metconazole, methasulfocarb, metiram, metominostrobin, mepanipyrim, metrafenone, myclobutanil, naftifine, neo-asozin (ferric methanearsonate), nuarimol, octhilinone, ofurace, orysastrobin, oxadixyl, oxolinic acid, oxpoconazole, oxycarboxin, oxytetracycline, penconazole, pencycuron, penflufen, penthiopyrad, pefurazoate, phosphorous acid and salts thereof, phthalide, picoxystrobin, piperalin, polyoxin, probenazole, prochloraz, procymidone, propamocarb, propamocarb-hydrochloride, propiconazole, propineb, proquinazid, prothiocarb, prothioconazole, pydiflumetofen, pyraclostrobin, pyrametostrobin, pyraoxystrobin, pyrazophos, pyribencarb, pyributicarb, pyrifenox, pyrimethanil, pyriofenone, pyrisoxazole, pyroquilon, pyrrolnitrin, quinomethionate, quinoxyfen, quintozene, sedaxane, silthiofam, simeconazole, spiroxamine, streptomycin, sulfur, tebuconazole, tebufloquin, tecloftalam, tecnazene, terbinafine, tetraconazole, thiabendazole, thifluzamide, thiophanate, thiophanate-methyl, thiram, tiadinil, tolclofos-methyl, tolylfluanid, tolnifanide, triadimefon, triadimenol, triazoxide, tricyclazole, tridemorph, triflumizole, tricyclazole, trifloxystrobin, triforine, trimorphamide, triticonazole, uniconazole, validamycin, valifenalate, vinclozolin, zineb, ziram, zoxamide, N-[4-[4-chloro-3-(trifluoromethyl)phenoxy]-2,5-dimethylphenyl]-N-ethyl-N-methylmethanimidamide, 5-chloro-6-(2,4,6-trifluorophenyl)-7-(4-methylpiperidin-1-yl)[1,2,4]triazolo[1,5a]pyrimidine (DPX-BAS600F), N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 4-fluorophenyl N-[1-[[[1-(4-cyanophenyl)ethyl]sulfonyl]-methyl]propyl]carbamate, N-[[(cyclopropylmethoxy)amino] [6-(difluoromethoxy)-2,3-difluorophenyl]methylene]benzeneacetamide, α-(methoxyimino)-N-methyl-2-[[[1-[3-(trifluoromethyl)phenyl]ethoxy]imino]methyl]benzeneacetamide, N-[4-[4-chloro-3-(trifluoromethyl)phenoxy]-2,5-dimethylphenyl]-N-ethyl-N-methylmethanimidamide, 2-[[[[3-(2,6-dichlorophenyl)-1-methyl-2-propen-1-ylidene]amino]oxy]methyl]-α-(methoxyimino)-N-methylbenzeneacetamide and 1-[(2-propenylthio)carbonyl]-2-(1-methyl-ethyl)-4-(2-methylphenyl)-5-amino-1H-pyrazol-3-one, 5-ethyl-6-octyl-[1,2,4]triazolo[1,5-a]pyrimidin-7-ylamine.

14. The composition of claim 13 wherein component (b) includes at least one compound selected from azoxystrobin, benzovindiflupyr, bixafen, chlorothalonil, copper hydroxide, cyproconazole, epoxiconazole, fenpropidin, fenpropimorph, fluindapyr, flutriafol, fluxapyroxad, mancozeb, metominostrobin, picoxystrobin, prothioconazole, pydiflumetofen, pyraclostrobin, tebuconazole and trifloxystrobin.

15. A composition comprising the composition of claim 1 and at least one additional component selected from the group consisting of surfactants, solid diluents and liquid diluents.

16. A method for protecting a plant or plant seed from diseases caused by fungal pathogens comprising applying a fungicidally effective amount of the composition of any one of claims 1 through 14 to the plant or plant seed.
